(19) United States
(12) Patent Application Publication
(10) Pub. No.: US 2004/0209930 A1
(43) Pub. Date: Oct. 21, 2004

(54) Synergistic methods and compositions for treating cancer

(76) Inventors: Joan M. Carboni, Yardley, PA (US); Warren W. Hurlburt, Mount Laurel, NJ (US); Marco M. Gottardis, Princeton, NJ (US); Francis Y. Lee, Yardley, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/814,199

(22) Filed: Mar. 31, 2004

Related U.S. Application Data
(63) Continuation-in-part of application No. 10/676,214, filed on Oct. 1, 2003, now abandoned.
(63) Continuation-in-part of application No. 10/677,067, filed on Oct. 1, 2003.
(60) Provisional application No. 60/415,416, filed on Oct. 2, 2002.

(51) Int. Cl. A61K 31/426

(52) U.S. Cl. 514/370

(57) ABSTRACT

Combination therapies using IGF1R inhibitors in combination with additional kinase inhibitors are described for the synergistic treatment of cancer.

### RELATED APPLICATIONS

This application is a Continuation-In-Part claiming priority benefit under Title 35 § 120 to U.S. application Ser. No. 10/676,214, filed Oct. 1, 2003, and Ser. No. 10/677,067, filed Oct. 1, 2003, both of which claim priority to U.S. Provisional Application No. 60/415,416, filed Oct. 2, 2002, the contents of which are herein incorporated by reference.

### FIELD OF THE INVENTION

The present invention relates to therapies for the treatment of cancer, specifically to synergistic methods for treating cancer using IGF1R inhibitors in combination with other kinase inhibitors.

### BACKGROUND OF THE INVENTION

Chemotherapy, the systemic administration of antineoplastic agents that travel throughout the body via the blood circulatory system, along with and often in conjunction with surgery and/or radiation treatment, has for years been widely utilized in the treatment of a wide variety of cancers.

Today, there are a variety of antineoplastic agents that have successfully been used in the treatment of cancer. However, the search continues for more efficacious and less toxic agents.

Kinases are a class of enzymes that have proven to be useful agents for the treatment of cancer. Kinases play a critical role in signal transduction for several cellular functions including cell proliferation, carcinogenesis, apoptosis, and cell differentiation (Plowman, G. D.; Ullrich, A.; Shawver, L. K.: Receptor Kinases As Targets For Drug Intervention. DN&P (1994) 7: 334-339). Inhibitors of these enzymes are actually useful for the treatment or prevention of a variety of proliferative diseases that are dependent on these enzymes. Strong epidemiologic evidence suggests that the overexpression or activation of receptor protein kinases leading to constitutive mitogenic signaling is an important factor in a growing number of human malignancies. Kinases that have been implicated in these processes include Abl, CDK's, EGF, EMT, FGF, FAK, Flk-1/KDR, HER-2, IGF-1R, IR, LCK, MET, PDGF, Src, MEK and VEGF (Traxler, P. M. Protein Kinase Inhibitors in Cancer Treatment. Exp. Opin. Ther. Patents (1997) 7: 571-588; incorporated herein by reference).

The IGF1R (insulin-like growth factor-1 receptor) affects cell mitogenesis, survival, transformation, and insulin-like activities by the binding of its ligands, IGF1 and IGF2. This receptor influences post natal growth physiology, and its activity has been associated with malignant disorders such as breast cancer. See, Ellis et al., Breast Cancer Res. Treat. 1998, 52, 175. The anti-apoptotic effect induced by the IGF1/IGF1R system correlates to the induction of chemoresistance in various tumors. See, Grothey et al., J. Cancer Res. Clin. Oncol., 1999, 125, 166-73. Accordingly, inhibitors of IGF1R are useful in the treatment of cancer, as evidenced in U.S. patent application Ser. No. 10/105,599. IGF1R inhibitors are useful as single agents and also in combination with other anticancer agents. See, Burtrum, et al., Cancer Research, Vol. 63, 8912-8921 (2003.)

Although combination chemotherapy has improved the response and survival rates of patients with hematological malignancies and some solid tumors, it is well known that anti-cancer drugs often bring on serious side effects that limit the doses physicians can administer. Accordingly, synergistic combination chemotherapy is especially desirable because the synergy between active ingredients allows for the use of smaller doses of one or both active ingredients, provides greater efficacy at the same doses, and/or prevents or delays the build-up of multi-drug resistance. There is a need in the art for synergistic chemotherapy regimens that are effective for the treatment of cancer with improved toxicity profiles.

### SUMMARY OF THE INVENTION

It has now been found, and this forms the subject of the present invention, that the efficacy of both IGF1R inhibitors and additional anticancer agents are considerably improved when they are administered in combination, resulting in methods for the synergistic treatment of cancer. Thus, the present invention is directed to methods for the synergistic treatment of cancer comprising administering to a mammal in need thereof a therapeutically effective amount of an anticancer agent in combination with a therapeutically effective amount of an IGF1R inhibitor in amounts sufficient to achieve synergistic results, optionally including treatment with an additional anticancer agent.

Advantages over previously disclosed methods include the ability of the instant combination of IGF1R inhibitors and other anticancer agents to be individually varied depending on the nature of the cancer cells to be treated. The therapeutic effect of the instant compositions is achieved with smaller amounts of either drug than would be required if such drugs were administered alone. This approach minimizes any non-mechanism-based adverse toxicity effects that might result from administration of an amount of an anticancer agent or an IGF1R inhibitor alone sufficient to achieve the same therapeutic effect. The synergistic methods of the present invention reduce the development of tumors, reduce tumor burden, or produce tumor regression in a mammalian host.

The present invention also includes pharmaceutical compositions comprising a therapeutically effective amount of an IGF1R inhibitor in combination with a therapeutically effective amount of a anticancer agent, wherein both the IGF1R inhibitor and the additional anticancer are present in amounts sufficient to achieve synergistic results in the treatment of cancer, in a pharmaceutically acceptable carrier.

### BRIEF DESCRIPTION OF THE DRAWINGS

FIG. 1 is an isobologram depicting the synergistic anticancer activity achieved when an IGF1R inhibitor (Compound 1) is administered in combination with an EGFR inhibitor, gefitinib, in IGF1R sal cells.

FIG. 2 is an isobologram depicting the synergistic anticancer activity achieved when an IGF1R inhibitor (Compound 1) is administered in combination with an EGFR inhibitor, gefinitib, in MCF-7 cells.

FIG. 3 is an isobologram depicting the synergistic anticancer activity achieved when an IGF1R inhibitor (Compound 1) is administered in combination with an EGFR inhibitor, gefitinib, in MDA-Pca-2b cells.

FIG. 4 is an isobologram depicting the synergistic anticancer activity achieved when an IGF1R inhibitor (Compound 1) is administered in combination with an EGFR inhibitor, cetuximab, in GEO cells.

FIG. 5 is an is an isobologram depicting the synergistic anticancer activity achieved when an IGF1R inhibitor (Compound 2) is administered in combination with an EGFR inhibitor, cetuximab, in GEO cells.

FIG. 6 is an isobologram depicting the synergistic anticancer activity achieved when an IGF1R inhibitor (Compound 2) is administered in combination with an EGFR inhibitor, gefitinib in RD1 cells.

FIG. 7 is an isobologram depicting the synergistic anticancer activity achieved when an IGF1R inhibitor (Compound 1) is administered in combination with an EGFR inhibitor, erlotinib, in MDA-Pca-2b cells.

FIG. 8 is an isobologram depicting the synergistic anticancer activity achieved when an IGF1R inhibitor (Compound 1) is administered in combination with an EGFR inhibitor, erlotinib, in MCF 7 cells.

FIG. 9 shows the effects of an IGF1R inhibitor (Compound 1) and an EGFR inhibitor, cetuximab, singly or in combination, on the growth of the GEO human colon carcinoma xenograft model in nude mice.

FIG. 10 is an isobologram demonstrating the synergistic effects observed when an IGF1R inhibitor is administered in combination with etoposide.

FIG. 11 is an isobologram demonstrating the synergistic effects observed when an IGF1R inhibitor is administered in combination with cisplatin.

FIG. 12 is an isobologram demonstrating the synergistic effects observed when an IGF1R inhibitor is administered in combination with paclitaxel.

FIG. 13 is a fraction plot for an IGF1R inhibitor ratio with a Src inhibitor in HT-29 cells, demonstrating the synergistic effect of the combination.

FIG. 14 is a fraction plot for an IGF1R inhibitor ratio with a Src inhibitor in Colo205 cells, demonstrating the synergistic effect of the combination.

FIG. 15 is an isobologram demonstrating the synergistic effects observed when an IGF1R inhibitor is administered in combination with a MEK inhibitor.

FIG. 16 is a fraction plot for an IGF1R inhibitor ratio with a pan Her inhibitor in Colo205 cells, demonstrating the synergistic effect of the combination.

FIG. 17 is a fraction plot for an IGF1R inhibitor ratio with a Src inhibitor in HT-29 cells, demonstrating the synergistic effect of the combination.

### DETAILED DESCRIPTION

The present invention is directed to synergistic methods for treating cancer comprising administering to a mammal in need of such treatment an IGF1R inhibitor in combination with an additional anticancer agent. The present invention provides methods for the synergistic treatment of a variety of cancers, including, but not limited to, the following:

carcinoma including that of the bladder (including accelerated and metastatic bladder cancer), breast, cervical, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma);

hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma;

hematopoietic tumors of myeloid lineage including acute and chronic myelogenous leukemias, myelodysplastic syndrome, myeloid leukemia, and promyelocytic leukemia;

tumors of the central and peripheral nervous system including astrocytoma, neuroblastoma, glioma, and schwannomas;

tumors of mesenchymal origin including fibrosarcoma, liposarcoma, rhabdomyosarcoma, and osteosarcoma; and

other tumors including melanoma, xenoderma pigmentosum, keratoactanthoma, seminoma, thyroid follicular cancer, and teratocarcinoma.

As used herein “synergistic result” or “synergy” refers to a therapeutic effect such that when administered in combination, the IGF1R inhibitor and the anticancer agent produce results that are significantly superior than the optimal efficacy obtained with either single agent alone.

As used herein, an “inhibitor” of a specific kinase receptor (such as an IGF1R inhibitor or and EGFR inhibitor, for example) is intended to mean a compound or a drug that is a biological molecule or a small molecule that directly or indirectly inhibits the receptor's activity or the related signal transduction pathway. Thus, compounds or drugs as used herein is intended to include both small molecules and biological molecules. In one aspect, inhibition refers to inhibition of the binding of receptor to a ligand such as. In another aspect, inhibition refers to inhibition of the kinase activity of specific receptor. Inhibitors include, for example, receptor specific ligands, small molecule receptor inhibitors, and receptor monoclonal antibodies.

“Biological molecules” according to the present invention, include all lipids and polymers of monosaccharides, amino acids, and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA. Biological molecules further include derivatives of any of the molecules described above. For example, derivatives of biological molecules include lipid and glycosylation derivatives of oligopeptides, polypeptides, peptides, and proteins. Derivatives of biological molecules further include lipid derivatives of oligosaccharides and polysaccharides, e.g., lipopolysaccharides. Most typically, biological molecules are antibodies, or functional equivalents of antibodies. Such functional equivalents include, for example, chimerized, humanized, and single chain antibodies as well as fragments thereof. Functional equivalents of antibodies also include polypeptides with amino acid sequences substantially the same as the amino acid sequence of the variable or hypervariable regions of the antibodies. An amino acid sequence that is substantially the same as another sequence, but that differs from the other sequence by means of one or more substitutions, additions, and/or deletions, is considered to be an equivalent sequence. Preferably, less than 50%, more preferably less than 25%, and still more preferably less than 10%, of the number of amino acid residues in a sequence are substituted for, added to, or deleted from the protein.

The functional equivalent of an antibody is preferably a chimerized or humanized antibody. A chimerized antibody comprises the variable region of a non-human antibody and the constant region of a human antibody. A humanized antibody comprises the hypervariable region (CDRs) of a non-human antibody.

The variable region other than the hypervariable region, e.g., the framework variable region, and the constant region of a humanized antibody are those of a human antibody. Suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates. Functional equivalents further include fragments of antibodies that have binding characteristics that are the same as, or are comparable to, those of the whole antibody. Suitable fragments of the antibody include any fragment that comprises a sufficient portion of the hypervariable (i.e., complementarity determining) region to bind specifically, and with sufficient affinity, to a kinase to inhibit growth of cells that express such receptors.

“Small molecule” as used herein refers to any molecule that is not a biological molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds.

As used herein, “anticancer” agent includes any biological or small molecule compound that is capable of inhibiting or preventing the growth and spread of neoplasms or malignant cells, other than an IGF1R inhibitor.

As used herein, the term “pan HER inhibitor” refers to a small molecule or biological compounds that inhibits both the HER1 and HER2 kinase. The human epidermal growth factor receptor (HER) family consists of four distinct receptor kinase referred to as HER1, HER2, HER3 and HER4. These kinases are also referred to as erbB1, erbB2, etc. HER1 is also commonly referred to as the EGF receptor. With the exception of HER3, these receptors have intrinsic protein kinase activity that is specific for tyrosine residues of phosphoacceptor proteins.

IGF1R inhibitors of the present invention include both small molecule and biological compounds. Such IGF1R inhibitors and methods for making them are described in U.S. application Ser. No. 10/263,448,U.S. patent application Ser. No. 10/751,798 filed Jan. 5, 2004; U.S. patent application Ser. No. 10/674,098 filed Sep. 29, 2003; U.S. Patent Application Ser. No. 60/546,814; WO03/048133; WO 01/25220; U.S. Pat. No. 6,337,338 (WO 00/35455); WO 02/102804; WO 02/092599; WO 03/024967; WO 03/035619; WO 03/035616; WO 03/018022;

WO 02/53596; Burtrum, et al. Cancer Research, Vol. 63, 8912-8921 (December 2003; Maloney et al., Cancer Research, Vol. 63, 5073-5083 (August 2003); and Long et al., Cancer Research, Vol. 55, 1006-1009 (March 1995); the disclosures of which are herein incorporated by reference in their entirety

In some embodiments of the present invention, the IGF1R inhibitor has the formula I:

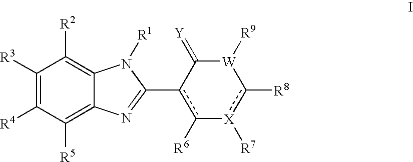

and includes its enantiomers, diastereomers, pharmaceutically acceptable salts, hydrates, prodrugs and solvates thereof;

wherein

X is N, C or a direct bond;

Y is O or S;

W is N, C, O, or S; provided that if W is O or S, R⁹ is absent;

R¹ is H, alkyl, or alkoxy;

R² and R⁹ are independently H or alkyl;

R³ is H, C₁₋₆ alkyl, alkenyl, alkynyl, cycloalkyl, heterocycloalkyl, halo, amino, —OR⁶⁰, —NO₂, —OH, —SR⁶⁰, —NR⁶⁰R⁶¹, —CN, —C(O)R⁶⁰, —CO₂R⁶⁰, —CONR⁶⁰R⁶¹, OCONR⁶⁰R⁶¹, —NR⁶²CONR⁶⁰R⁶¹, —NR⁶⁰SO₂R⁶¹, —SO₂NR⁶⁰R⁶¹, —SO₂R⁶³, —C(NR⁶²)NR⁶⁰R⁶¹, —C(NH⁶²)-morpholine, aryl, heteroaryl, —(CH₂)ₙC(O)₂—R⁶⁰, —NR⁶⁰R⁶¹—(CH₂)ₙOR⁶⁰, —(CH₂)ₙNR⁶⁰R⁶¹, —(CH₂)ₙSR⁶⁰, —(CH₂)ₙ aryl, —(CH₂)ₙ heteroaryl, or —(CH₂)ₙ heterocycloalkyl, wherein n is 1 to 3:

R⁴ is H, halo, alkyl or haloalkyl;

R⁵ is H, alkyl, halo, or aryl;

R⁶, R⁷, and R⁸ are each independently —NH-Z-aryl or —NH-Z-heteroaryl wherein Z is C₁-C₄ alkyl, alkenyl, or alkynyl; Z optionally having one or more hydroxy, thiol, alkoxy, thioalkoxy, amino, halo, NR⁶⁰SO₂R⁶¹ groups; Z optionally incorporating one or more groups selected from the group consisting of CO, CNOH, CNOR⁶⁰, CNNR⁶⁰, CNNCOR⁶⁰ and CNNSO₂R⁶⁰;

R⁶⁰, R⁶¹R⁶², and R⁶³ are independently selected from the group consisting of H, alkyl, alkenyl, alkynyl, cycloalkyl, cycloalkylalkyl, hydroxy, alkoxy, aryl, heteroaryl, heteroarylalkyl, and alkyl-R²⁵;

R²⁵ is hydrogen, alkenyl, hydroxy, thiol, alkoxy, thioalkoxy, amino, alkylamino, dialkylamino, aryl, heteroaryl, cyano, halo, sulfoxy, sulfonyl, —NR³⁰COOR³¹, —NR³⁰C(O)R³¹, —NR³⁰SO₂R³¹, —C(O)NR³⁰R³¹, heteroaryl or heterocycloalkyl; and

R³⁰ and R³¹ are, independently, hydrogen, alkyl, or cycloalkyl.

In some embodiments of the present invention, R¹ is H, alkyl or alkoxy, R² is H; R³ is H, alkyl, —CN, halo, —C(O)R⁶⁰—C(O)NR⁶⁰R⁶¹, —S(O)₂R⁶³, piperazine, piperidine, morpholine, triazole, imidazole, wherein the piperazine, piperidine, morpholine, triazole, or imidazole is substituted with H, alkyl, —NHC(O)alkyl, —NHC(O)₂alkyl, —NHC(O)alkoxy, —O—(CH₂)ₙR⁶⁴ wherein R⁶⁴ is hydroxy, alkoxy, morpholine, or tetrahydropyrimidine; and R⁶ is —NH-Z-phenyl; —NH-Z-imidazole; or —NH-Z-pyrazole wherein Z is C1 to C2 alkyl.

According to some embodiments of the present invention, the IGF1R inhibitor has the formula IA:

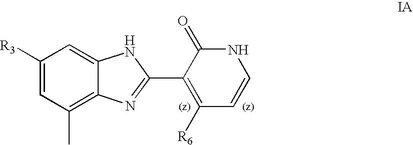

wherein R³ is an unsubstituted or substituted morpholine, piperazine or piperidine and R⁶ is —NHZ-aryl or —NHZ-heteroaryl.

In some embodiments of the present invention, the IGF1R inhibitor is selected from the group consisting of:

(S)-4-(2-Hydroxy-1-phenyl-ethylamino)-3-(6-imidazol-1-yl-4-methyl-1H-benzimidazol-2-yl)-1H-pyridin-2-one;

(±)-4-[2-Hydroxy-2-(3-iodo-phenyl)-ethylamino]-3-(6-imidazol-1-yl-4-methyl-1H-benzimidazol-2-yl)-1H-pyridin-2-one;

(±)-4-[2-(3-Chloro-phenyl)-2-hydroxy-ethylamino]-3-(6-imidazol-1-yl-4-methyl-1H-benzimidazol-2-yl)-1H-pyridin-2-one;

(±)-4-[2-(3-Bromo-phenyl)-2-hydroxy-ethylamino]-3-(6-imidazol-1-yl-4-methyl-1H-benzimidazol-2-yl)-1H-pyridin-2-one;

(S)-4-[2-(2-Chloro-phenyl)-1-hydroxymethyl-ethylamino]-3-(6-imidazol-1-yl-4-methyl-1H-benzimidazol-2-yl)-1H-pyridin-2-one;

(S)-4-[2-(3-Chloro-phenyl)-1-hydroxymethyl-ethylamino]-3-(6-imidazol-1-yl-4-methyl-1H-benzimidazol-2-yl)-1H-pyridin-2-one;

(S)-4-[2-(4-Chloro-phenyl)-1-hydroxymethyl-ethylamino]-3-(6-imidazol-1-yl-4-methyl-1H-benzimidazol-2-yl)-1H-pyridin-2-one;

(S)-4-[2-(2-Bromo-phenyl)-1-hydroxymethyl-ethylamino]-3-(6-imidazol-1-yl-4-methyl-1H-benzimidazol-2-yl)-1H-pyridin-2-one;

(S)-4-[2-(3-Bromo-phenyl)-1-hydroxymethyl-ethylamino]-3-(6-imidazol-1-yl-4-methyl-1H-benzimidazol-2-yl)-1H-pyridin-2-one;

(±)-4-(1-Hydroxymethyl-2-pentafluorophenyl-ethylamino)-3-(6-imidazol-1-yl-4-methyl-1H-benzimidazol-2-yl)-1H-pyridin-2-one;

(S)-4-(1-Hydroxymethyl-2-pyridin-4-yl-ethylamino)-3-(6-imidazol-1-yl-4-methyl-1H-benzimidazol-2-yl)-1H-pyridin-2-one;

(S)-4-[1-Hydroxymethyl-2-(2-naphthalenyl)-ethylamino]-3-(6-imidazol-1-yl-4-methyl-1H-benzimidazol-2-yl)-1H-pyridin-2-one;

3-(6-Imidazol-1-yl-4-methyl-1H-benzimidazol-2-yl)-4-(pyridin-2-ylmethoxy)-1H-pyridin-2-one;

(±)-4-[2-(3-Bromo-phenyl)-2-fluoro-ethylamino]-3-(6-imidazol-1-yl-4-methyl-1H-benzimidazol-2-yl)-1H-pyridin-2-one;

(S)-2-[4-(1-Hydroxymethyl-2-phenyl-ethylamino)-2-oxo-1,2-dihydro-pyridin-3-yl]-7-methyl-3H-benzimidazole-5-carbonitrile;

(±)-2-{4-[2-(3-Chloro-phenyl)-2-hydroxy-ethylamino]-2-oxo-1,2-dihydro-pyridin-3-yl}-7-methyl-3H-benzimidazole-5-carbonitrile;

(S)-2-{4-[2-(3-Chloro-phenyl)-1-hydroxymethyl-ethylamino]-2-oxo-1,2-dihydro-pyridin-3-yl}-7-methyl-3H-benzimidazole-5-carbonitrile;

(±)-2-{4-[2-(3-Bromo-4-methoxy-phenyl)-2-hydroxy-ethylamino]-2-oxo-1,2-dihydro-pyridin-3-yl}-7-methyl-3H-benzimidazole-5-carbonitrile;

(±)-2-{4-[2-(3-Fluoro-phenyl)-2-hydroxy-ethylamino]-2-oxo-1,2-dihydro-pyridin-3-yl}-7-methyl-3H-benzimidazole-5-carbonitrile;

(±)-2-{4-[2-(3-Bromo-phenyl)-2-hydroxy-ethylamino]-2-oxo-1,2-dihydro-pyridin-3-yl}-7-methyl-3H-benzimidazole-5-carbonitrile;

(S)-2-[4-(2-Hydroxy-2-phenyl-ethylamino)-2-oxo-1,2-dihydro-pyridin-3-yl]-7-methyl-3H-benzimidazole-5-carbonitrile;

(±)-3-(1H-Benzimidazol-2-yl)-4-[2-(3-bromo-phenyl)-2-hydroxy-ethylamino]-1H-pyridin-2-one;

(S)-3-(1H-Benzimidazol-2-yl)-4-(1-hydroxymethyl-2-phenyl-ethylamino)-1H-pyridin-2-one;

(±)-3-(1H-Benzimidazol-2-yl)-4-[2-(3-bromo-4-methoxy-phenyl)-2-hydroxy-ethylamino]-1H-pyridin-2-one;

(S)-4-{2-[4-(1-hydroxymethyl-2-phenyl-ethylamino)-2-oxo-1,2-dihydro-pyridin-3-yl]-7-methyl-3H-benzimidazol-5-yl}-piperazine-1-carboxylic acid isopropylamide;

(S)-4-{2-[4-(1-hydroxymethyl-2-phenyl-ethylamino)-2-oxo-1,2-dihydro-pyridin-3-yl]-7-methyl-3H-benzimidazol-5-yl}-piperazine-1-carboxylic acid ethylamide;

(S)-4-(1-Hydroxymethyl-2-phenyl-ethylamino)-3-{4-methyl-6-[4-(1-phenyl-methanoyl)-piperazin-1-yl]-1H-benzimidazol-2-yl}-1H-pyridin-2-one;

(S)-4-(1-Hydroxymethyl-2-phenyl-ethylamino)-3-[6-(4-isopropyl-piperazin-1-yl)-4-methyl-1H-benzimidazol-2-yl]-1H-pyridin-2-one;

(S)-3-[6-(4-Benzyl-piperazine-1-yl)-4-methyl-1H-benzimidazol-2-yl]-4-(1-hydroxymethyl-2-phenyl-ethylamino)-1H-pyridin-2-one;

(±)-3-[6-(4-Acetyl-piperazine-1-yl)-4-methyl-1H-benzimidazol-2-yl]-4-[2-(3-chloro-phenyl)-2-hydroxy-ethylamino]-1H-pyridin-2-one;

(±)-4-[2-(3-Chloro-phenyl)-2-hydroxy-ethylamino]-3-(4-methyl-6-piperazin-1-yl-1H-benzimidazol-2-yl)-1H-pyridin-2-one;

(±)-4-[2-(3-Chloro-phenyl)-2-hydroxy-ethylamino]-3-[6-(4-isopropyl-piperazine-1-yl)-4-methyl-1H-benzimidazol-2-yl]-1H-pyridin-2-one;

(S)-6-(1-Hydroxymethyl-2-phenyl-ethylamino)-5-(6-imidazol-1-yl-4-methyl-1H-benzimidazol-2-yl)-3H-pyrimidin-4-one;

(S)-2-[6-Chloro-5-(6-imidazol-1-yl-4-methyl-1H-benzimidazol-2-yl)-pyrimidin-4-ylamino]-3-phenyl-propan-1-ol;

(S)-4-(2-Hydroxy-2-phenyl-ethylamino)-3-(6-imidazol-1-yl-4-methyl-1H-benzimidazol-2-yl)-1H-pyridin-2-one;

(R)-4-(2-Hydroxy-2-phenyl-ethylamino)-3-(6-imidazol-1-yl-4-methyl-1H-benzimidazol-2-yl)-1H-pyridin-2-one;

(1S,2R)-4-(1-Hydroxy-indan-2-ylamino)-3-(6-imidazol-1-yl-4-methyl-1H-benzimidazol-2-yl)-1H-pyridin-2-one;

(±)-4-[2-Hydroxy-2-(3-hydroxy-phenyl)-ethylamino]-3-(6-imidazol-1-yl-4-methyl-1H-benzimidazol-2-yl)-1H-pyridin-2-one;

(S)-4-(2-Hydroxy-2-pyridin-2-yl-ethylamino)-3-(6-imidazol-1-yl-4-methyl-1H-benzimidazol-2-yl)-1H-pyridin-2-one;

(±)-N-(3-{1-Hydroxy-2-[3-(6-imidazol-1-yl-4-methyl-1H-benzimidazol-2-yl)-2-oxo-1,2-dihydro-pyridin-4-ylamino]-ethyl}-phenyl)-methanesulfonamide;

(±)-4-[2-(3-Fluoro-phenyl)-2-hydroxy-ethylamino]-3-(6-imidazol-1-yl-4-methyl-1H-benzimidazol-2-yl)-1H-pyridin-2-one;

(±)-4-[2-(3-Chloro-4-fluoro-phenyl)-2-hydroxy-ethylamino]-3-(6-imidazol-1-yl-4-methyl-1H-benzimidazol-2-yl)-1H-pyridin-2-one;

(S)-4-[2-(3-Fluoro-phenyl)-1-hydroxymethyl-ethylamino]-3-(6-imidazol-1-yl-4-methyl-1H-benzimidazol-2-yl)-1H-pyridin-2-one;

(±)-4-[2-(3-Chloro-phenyl)-2-hydroxy-ethylamino]-3-(6-imidazol-1-yl-1H-benzimidazol-2-yl)-1H-pyridin-2-one;

(±)-4-[2-(3-Bromo-4-methoxy-phenyl)-2-hydroxy-ethylamino]-3-(6-imidazol-1-yl-4-methyl-1H-benzimidazol-2-yl)-1H-pyridin-2-one;

(S)-4-[2-(3-Bromo-phenyl)-2-hydroxy-ethylamino]-3-(6-imidazol-1-yl-4-methyl-1H-benzimidazol-2-yl)-1H-pyridin-2-one;

(S)-4-[2-(3-Chloro-phenyl)-2-hydroxy-ethylamino]-3-(6-imidazol-1-yl-4-methyl-1H-benzimidazol-2-yl)-1H-pyridin-2-one;

(R)-4-[2-(3-Chloro-phenyl)-2-hydroxy-ethylamino]-3-(6-imidazol-1-yl-4-methyl-1H-benzimidazol-2-yl)-1H-pyridin-2-one;

(±)-4-[2-(3-Chloro-4-methoxy-phenyl)-2-hydroxy-ethylamino]-3-(6-imidazol-1-yl-4-methyl-1H-benzimidazol-2-yl)-1H-pyridin-2-one;

(±)-(2-Chloro-4-{1-hydroxy-2-[3-(6-imidazol-1-yl-4-methyl-1H-benzimidazol-2-yl)-2-oxo-1,2-dihydro-pyridin-4-ylamino]-ethyl}-phenyl)-carbamic acid methyl ester;

(s)-4-(1-Hydroxymethyl-2-phenyl-ethylamino)-3-[4-methyl-6-(4-methyl-piperazin-1-yl)-1H-benzimidazol-2-yl]-1H-pyridin-2-one;

(S)-4-(1-Hydroxymethyl-2-phenyl-ethylamino)-3-[4-methyl-6-(4-n-butyl-piperazin-1-yl)-1H-benzimidazol-2-yl]-1H-pyridin-2-one;

(S)-3-{6-[4-(2-Hydroxy-ethyl)-piperazin-1-yl]-4-methyl-1H-benzimidazol-2-yl}-4-(1-hydroxymethyl-2-phenyl-ethylamino)-1H-pyridin-2-one;

(S)-4-{2-[4-(1-Hydroxymethyl-2-phenyl-ethylamino)-2-oxo-1,2-dihydro-pyridin-3-yl]-7-methyl-3H-benzimidazol-5-yl}-piperazine-1-carboxylic acid amide;

(±)-4-[2-(3-Chloro-phenyl)-2-hydroxy-ethylamino]-3-(4-methyl-6-piperazin-1-yl-1H-benzimidazol-2-yl)-1H-pyridin-2-one;

(±)-4-[2-(3-Chloro-phenyl)-2-hydroxy-ethylamino]-3-[6-(4-ethyl-piperazin-1-yl)-4-methyl-1H-benzimidazol-2-yl]-1H-pyridin-2-one;

(±)-4-[2-(3-Chloro-phenyl)-2-hydroxy-ethylamino]-3-{6-[4-(2-hydroxy-ethyl)piperazin-1-yl]-4-methyl-1H-benzimidazol-2-yl}-1Hpyridin-2-one;

(±)-4-[2-(3-Chloro-phenyl)-2-hydroxy-ethylamino]-3-(4-methyl-6-morpholin-4-yl-1H-benzimidazol-2-yl)-1H-pyridin-2-one;

(±)-4-[2-(3-Bromo-phenyl)-2-hydroxy-ethylamino]-3-(4-methyl-6-morpholin-4-yl-1H-benzimidazol-2-yl)-1H-;

(±)-4-[2-(3-Bromo-4-methoxy-phenyl)-2-hydroxy-ethylamino]-3-(4-methyl-6-morpholin-4-yl-1H-benzimidazol-2-yl)-1H-pyridin-2-one;

(±)-4-[2-(3-Bromo-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;

(±)-4-[2-(3-Bromo-phenyl)-2-hydroxy-ethylamino]-3-(4-methyl-6-piperazin-1-yl-1H-benzimidazol-2-yl}-1H-pyridin-2-one;

(±)-4-[2-(3-Bromo-phenyl)-2-hydroxy-ethylamino]-3-(4-methyl-6-piperazin-1-yl-1H-benzimidazol-2-yl}-1H-pyridin-2-one;

(±)-3-[6-(4-Acetyl-piperazin-1-yl)-4-methyl-1H-benzimidazol-2-yl]-4-[2-(3-bromo-phenyl)-2-hydroxy-ethylamino]-1H-pyridin-2-one;

(S)-4-(1-hydroxymethyl-2-phenyl-ethylamino)-3-[4-methyl-6-(2-morpholin-4-yl-ethylamino)-1H-benzimidazol-2-yl]-1H-pyridin-2-one;

(±)-6-[2-(3-Chloro-phenyl)-2-hydroxy-ethylamino]-5-(6-imidazol-1-yl-4-methyl-1H-benzimidazol-2-yl)-3H-pyrimidin-4-one;

(±)-4-[2-(3-Chloro-phenyl)-2-hydroxy-ethylamino]-3-[6-(1-hydroxy-1-methyl-ethyl)-4-methyl-1H-benzimidazol-2-yl]-1H-pyridin-2-one;

(±)-3-(6-Aminomethyl-4-methyl-1H-benzimidazol-2-yl)-4-[2-(3-chloro-phenyl)-2-hydroxy-ethylamino]-1H-pyridin-2-one;

(±)-4-[2-(3-Chloro-phenyl)-2-hydroxy-ethylamino]-3-(6-hydroxymethyl-4-methyl-1H-benzimidazol-2-yl)-1H-pyridin-2-one;

(S)-4-(1-Benzyl-2-hydroxy-ethylamino)-3-(4-methyl-6-morpholin-4-yl-1H-benzimidazol-2-yl)-1H-pyridin-2-one; and

(S)-4-(1-Benzyl-2-hydroxy-ethylamino)-3-(4-methyl-6-piperidin-1-yl-1H-benzimidazol-2-yl)-1H-pyridin-2-one;

(S)-4-(1-Benzyl-2-hydroxy-ethylamino)-3-(4-methyl-6-piperidin-1-yl-1H-benzimidazol-2-yl)-1H-pyridin-2-one;

4-[2-(3-Chloro-4-methylsulfanyl-phenyl)-2-hydroxy-ethylamino]-3-(4-methyl-6-piperazin-1-yl-1H-benzoimidazol-2-yl)-1H-pyridin-2-one;

4-[2-(3-Chloro-4-fluoro-phenyl)-2-hydroxy-ethylamino]-3-(4-methyl-6-piperazin-1-yl-1H-benzoimidazol-2-yl)-1H-pyridin-2-one;

3-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-propionitrile;

4-[2-(3-Chloro-phenyl)-2-hydroxy-ethylamino]-3-{6-[4-(2-methanesulfonyl-ethyl)-piperazin-1-yl]-4-methyl-1H-benzoimidazol-2-yl}-1H-pyridin-2-one;

3-[4-(2-{4-[2-(3-Bromo-4-methoxy-phenyl)-2-hydroxy-ethylamino]-2-oxo-1,2-dihydro-pyridin-3-yl}-3H-benzoimidazol-5-yl)-7-methyl-piperazin-1-yl]-propionitrile;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid 2-fluoro-ethyl ester;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid 2-methoxy-ethyl ester;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid tert-butyl ester;

4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazine-1-carboxylic acid prop-2-ynyl ester;

4-(2-{4-[2-(3-Bromo-4-methoxy-phenyl)-2-hydroxy-ethylamino]-2-oxo-1,2-dihydro-pyridin-3-yl}-7-methyl-3H-benzoimidazol-5-yl)-piperazine-1-carboxylic acid tert-butyl ester;

(S)-4-(2-{4-[2-(3-Bromo-4-methoxy-phenyl)-2-hydroxy-ethylamino]-2-oxo-1,2-dihydro-pyridin-3-yl}-7-methyl-3H-benzimidazol-5-yl)-piperazine-1-carboxylic acid ethyl ester;

4-[2-(3-Chloro-4-methoxy-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoro-ethyl)-piperazin-1-yl]-4-methyl-1H-benzoimidazol-2-yl}-1H-pyridin-2-one;

4-[2-(3-Chloro-4-fluoro-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Bromo-4-methoxy-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{4-methyl-6-[4-(3,3,3-trifluoro-propyl)-piperazin-1-yl]-1H-benzoimidazol-2-yl}-1H-pyridin-2-one;

4-[2-(3-Chloro-phenyl)-2-hydroxy-ethylamino]-3-{6-[4-(3-fluoro-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{4-methyl-6-[4-(3,4,4-trifluoro-but-3-enyl)-piperazin-1-yl]-1H-benzoimidazol-2-yl}-1H-pyridin-2-one;

4-[2-(3-Chloro-phenyl)-2-hydroxy-ethylamino]-3-{6-[4-(3-fluoro-2-hydroxy-propyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-hydroxy-2-methyl-propyl)-piperazin-1-yl]-4-methyl-1H-benzoimidazol-2-yl}-1H-pyridin-2-one;

(S)-4-[2-(3-Chloro-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;

(S)-4-[2-(3-Bromo-4-methoxy-phenyl)-2-hydroxy-ethylamino]-3-{6-[4-(2-hydroxy-ethyl)-piperazin-1-yl]-4-methyl-1H-benzimidazol-2-yl}-1H-pyridin-2-one;

[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetonitrile;

4-[2-(3-Chloro-phenyl)-2-hydroxy-ethylamino]-3-{6-[4-(4-fluoro-butyryl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2,2-difluoro-acetyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-methanesulfonyl-acetyl)-piperazin-1-yl]-4-methyl-1H-benzoimidazol-2-yl}-1H-pyridin-2-one;

3-[6-(4-Acetyl-piperazin-1-yl)-4-methyl-1H-benzoimidazol-2-yl]-4-[2-(3-chloro-phenyl)-2-hydroxy-ethylamino]-1H-pyridin-2-one;

4-[2-(3-Chloro-phenyl)-2-hydroxy-ethylamino]-3-(4-methyl-6-{4-[2-(1-oxo-1l4-thiomorpholin-4-yl)-acetyl]-piperazin-1-yl}-1H-benzoimidazol-2-yl)-1H-pyridin-2-one;

4-[2-(3-Chloro-phenyl)-2-hydroxy-ethylamino]-3-(6-{4-[2-(1,1-dioxo-116-thiomorpholin-4-yl)-acetyl]-piperazin-1-yl}-4-methyl-1H-benzoimidazol-2-yl)-1H-pyridin-2-one;

4-[2-(3-Chloro-phenyl)-2-hydroxy-ethylamino]-3-{4-methyl-6-[4-(2-thiomorpholin-4-yl-acetyl)-piperazin-1-yl]-1H-benzoimidazol-2-yl}-1H-pyridin-2-one;

4-[2-(3-Chloro-phenyl)-2-hydroxy-ethylamino]-3-{6-[4-(2-methanesulfinyl-acetyl)-piperazin-1-yl]-4-methyl-1H-benzoimidazol-2-yl}-1H-pyridin-2-one;

4-[2-(3-Chloro-phenyl)-2-hydroxy-ethylamino]-3-{6-[4-(2-methoxy-acetyl)-piperazin-1-yl]-4-methyl-1H-benzoimidazol-2-yl}-1H-pyridin-2-one;

4-[2-(3-Chloro-phenyl)-2-hydroxy-ethylamino]-3-{4-methyl-6-[4-(2-methylsulfanyl-acetyl)-piperazin-1-yl]-1H-benzoimidazol-2-yl}-1H-pyridin-2-one;

3-{6-[4-(2-Chloro-acetyl)-piperazin-1-yl]-4-methyl-1H-benzoimidazol-2-yl}-4-[2-(3-chloro-phenyl)-2-hydroxy-ethylamino]-1H-pyridin-2-one;

(S)-4-(2-{4-[2-(3-Bromo-4-methoxy-phenyl)-2-hydroxy-ethylamino]-2-oxo-1,2-dihydro-pyridin-3-yl}-7-methyl-3H-benzimidazol-5-yl)-piperazine-1-carbaldehyde;

(S)-4-(2-{4-[2-(3-Chloro-phenyl)-2-hydroxy-ethylamino]-2-oxo-1,2-dihydro-pyridin-3-yl}-7-methyl-3H-benzimidazol-5-yl)-piperazine-1-carbaldehyde;

(S)-4-[2-(3-Chloro-phenyl)-2-hydroxy-ethylamino]-3-(4-methyl-6-morpholin-4-yl-1H-benzoimidazol-2-yl)-1H-pyridin-2-one;

4-[2-(3-Bromo-4-methoxy-phenyl)-2-hydroxy-ethylamino]-3-(4-methyl-6-morpholin-4-yl-1H-benzoimidazol-2-yl)-1H-pyridin-2-one;

4-[2-(3-Chloro-4-fluoro-phenyl)-2-hydroxy-ethylamino]-3-(4-methyl-6-morpholin-4-yl-1H-benzoimidazol-2-yl)-1H-pyridin-2-one;

4-[2-(3-Chloro-4-methoxy-phenyl)-2-hydroxy-ethylamino]-3-(4-methyl-6-morpholin-4-yl-1H-benzoimidazol-2-yl)-1H-pyridin-2-one;

4-[2-(7-Bromo-2,3-dihydro-benzofuran-5-yl)-2-hydroxy-ethylamino]-3-(4-methyl-6-morpholin-4-yl-1H-benzoimidazol-2-yl)-1H-pyridin-2-one;

4-[2-(3-Chloro-phenyl)-2(S)-hydroxy-ethylamino]-3-[4-methyl-6-[2(S),6(R)-dimethyl-morpholine-4-yl]-1H-benzoimidazol-2-yl]-1H-pyridine-2-one;

4-[2-(3-Bromo-4-methoxy-phenyl)-2(S)-hydroxy-ethylamino]-3-[4-methyl-6-[2(S),6(R)-dimethyl-morpholine-4-yl]-1H-benzoimidazol-2-yl]-1H-pyridine-2-one;

4-[2-(3-Chloro-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(7-Bromo-2,3-dihydro-benzofuran-4-yl)-(S)-2-hydroxy-ethylamino]-3-{6-[(R)-2-fluoromethyl-morpholin-4-yl]-4-methyl-1H-benzimidazol-2-yl}-1H-pyridin-2-one and 4-[2-(7-bromo-2,3-dihydro-benzofuran-4-yl)-(S)-2-hydroxy-ethylamino]-3-{6-[(S)-2-fluoromethyl-morpholin-4-yl]-4-methyl-1H-benzimidazol-2-yl}-1H-pyridin-2-one;

4-[2-(3-Chloro-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-hydroxymethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-hydroxy-methyl-morpholin-4-yl]-4-methyl-1H-benzimidazol-2-yl}-1H-pyridin-2-one;

4-[2-(3-Chloro-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-methyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-methyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-methyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-methyl-morpholin-4-yl]-4-methyl-1H-benzimidazol-2-yl}-1H-pyridin-2-one;

4-[2-(3-Chloro-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-chloro-phenyl)-(S)-2-hydroxy-ethylamino]-3-{6-[(S)-2-methoxy-methyl-morpholin-4-yl]-4-methyl-1H-benzimidazol-2-yl}-1H-pyridin-2-one;

4-[2-(3-Bromo-4-methoxy-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-bromo-4-methoxy-phenyl)-(S)-2-hydroxy-ethylamino]-3-{6-[(S)-2-methoxymethyl-morpholin-4-yl]-4-methyl-1H-benzimidazol-2-yl}-1H-pyridin-2-one;

4-[2-(3-Chloro-4-methoxy-phenyl)-(S)-2-hydroxy-ethylamino]-3-{6-[(R)-2-methoxymethyl-morpholin-4-yl]-4-methyl-1H-benzimidazol-2-yl}-1H-pyridin-2-one and 4-[2-(3-chloro-4-methoxy-phenyl)-(S)-2-hydroxy-ethylamino]-3-{6-[(S)-2-methoxymethyl-morpholin-4-yl]-4-methyl-1H-benzimidazol-2-yl}-1H-pyridin-2-one;

4-[2-(3-Chloro-phenyl)-2(S)-hydroxy-ethyl amino]-3-[4-methyl-6-(4-methyl-piperazin-1-yl)-1H-benzoimidazol-2-yl]-1H-pyridine-2-one;

4-[2-(3-Bromo-4-methoxy-phenyl)-2(S)-hydroxy-ethylamino]-3-[4-methyl-6-(4-methyl-piperazin-1-yl)-1H-benzoimidazol-2-yl]-1H-pyridine-2-one;

4-[2-(3-Chloro-phenyl)-2-hydroxy-ethylamino]-3-{6-[4-(acetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-hydroxyacetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoroacetamido)-piperidin-1-yl]-4-methyl-1H-benzoimidazol-2-yl}-1H-pyridin-2-one;

4-[2-(3-Bromo-4-methoxy-phenyl)-2-hydroxy-ethylamino]-3-{6-[4-(acetamido)-piperidin-1-yl]-4-methyl-1H-benzoimidazol-2-yl}-1H-pyridin-2-one;

4-[2-(3-Bromo-phenyl)-2-hydroxy-ethylamino]-3-{6-[4-(2-hydroxyacetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoroacetamido)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-methoxyethoxycarbamoyl)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(methoxycarbamoyl)-piperidin-1-yl]-4-methyl-1H-benzoimidazol-2-yl}-1H-pyridin-2-one;

4-[2-(3-Chloro-phenyl)-2-hydroxy-ethylamino]-3-{6-[4-(2-fluoroethoxy carbamoyl)-piperidin-1-yl]-4-methyl-1H-benzoimidazol-2-yl}-1H-pyridin-2-one;

(S)-4-[2-(3-Chloro-phenyl)-2-hydroxy-ethylamino]-3-[4-methyl-6-(2-morpholin-4-yl-ethoxy)-1H-benzimidazol-2-yl]-1H-pyridin-2-one;

(S)-4-[2-(3-Bromo-4-methoxy-phenyl)-2-hydroxy-ethylamino]-3-[4-methyl-6-(2-morpholin-4-yl-ethoxy)-1H-benzimidazol-2-yl]-1H-pyridin-2-one;

(S)-4-[2-(3-Chloro-phenyl)-2-hydroxy-ethylamino]-3-[4-methyl-6-(2-methoxy-ethoxy)-1H-benzimidazol-2-yl]-1H-pyridin-2-one;

(S)-4-[2-(3-Chloro-phenyl)-2-hydroxy-ethylamino]-3-[4-methyl-6-(2-hydroxy-ethoxy)-1H-benzimidazol-2-yl]-1H-pyridin-2-one;

(S)-4-[2-(3-Bromo-4-methoxy-phenyl)-2-hydroxy-ethylamino]-3-[4-methyl-6-(2-morpholin-4-yl-propoxy)-1H-benzimidazol-2-yl]-1H-pyridin-2-one;

(S)-4-[2-(3-Chloro-phenyl)-2-hydroxy-ethylamino]-3-[4-methyl-6-(2-morpholin-4-yl-propoxy)-1H-benzimidazol-2-yl]-1H-pyridin-2-one;

(S)-3-(4-Bromo-6-morpholin-4-ylmethyl-1H-benzimidazol-2-yl)-4-[2-(3-chloro-phenyl)-2-hydroxy-ethylamino]-1H-pyridin-2-one;

(S)-3-[4-Bromo-6-(4-methyl-piperazin-1-ylmethyl-1H-benzimidazol-2-yl)-4-[2-(3-chloro-phenyl)-2-hydroxy-ethylamino]-1H-pyridin-2-one;

(S)-4-[2-(3-Chloro-phenyl)-2-hydroxy-ethylamino]-3-[4-methyl-6-(4-methyl-piperazin-1-ylmethyl)-1H-benzimidazol-2-yl]-1H-pyridin-2-one;

4-[2-(3-Chloro-phenyl)-2(S)-hydroxy-ethylamino]-3-[4-methyl-6-(1,4,5,6-tetrahydropyrimidine-1-yl)-1H-benzoimidazol-2-yl]-1H-pyridine-2-one; and

4-[2-(4-Methoxy-3-Chloro-phenyl)-2(S)-hydroxy-ethylamino]-3-[4-methyl-6-(1,4,5,6-tetrahydropyrimidine-1-yl)-1H-benzoimidazol-2-yl]-1H-pyridine-2-one;

4-[2-(3-Chloro-4-methoxy-phenyl)-2-hydroxy-ethylamino]-3-(4-methyl-6-morpholin-4-yl-1H-benzoimidazol-2-yl)-1,5-dihydro-pyrrol-2-one;

4-[2-(3-Bromo-4-methoxy-phenyl)-2-hydroxy-ethylamino]-3-(4-methyl-6-morpholin-4-yl-1H-benzoimidazol-2-yl)-1,5-dihydro-pyrrol-2-one;

(S)-4-[2-(3-Chloro-phenyl)-2-hydroxy-ethylamino]-3-(4-methyl-6-morpholin-4-yl-1H-benzoimidazol-2-yl)-1,5-dihydro-pyrrol-2-one;

(S,S and S,R)-4-[2-(3-Chloro-phenyl)-2-hydroxy-ethylamino]-5-methyl-3-(4-methyl-6-morpholin-4-yl-1H-benzoimidazol-2-yl)-1,5-dihydro-pyrrol-2-one;

[1-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperidin-4-yl]-carbamic acid tetrahydro-furan-3-ylmethyl ester;

[1-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperidin-4-yl]-carbamic acid 2-methoxy-propyl ester;

(S)-2-[4-(2-{4-[2-(3-Chloro-phenyl)-2-hydroxy-ethylamino]-2-oxo-1,2-dihydro-pyridin-3-yl}-7-methyl-3H-benzoimidazol-5-yl)-piperazin-1-yl]-acetamide Bis hydrochloride;

(S)-4-[2-(3-Chloro-phenyl)-2-hydroxy-ethylamino]-3-{6[4-(2-methyoxy-ethyl)-piperazin-1-yl]-4-methyl-1H-benzoimidazol-2-yl 1H-pyridin-2-one bis-hydrochloride;

(S)-4-[2-(3-Bromo-phenyl)-2-hydroxy-ethylamino]-3-6[4-(2-methyoxy-ethyl)-piperazin-1-yl]-4-methyl-1H-benzoimidazol-2-yl-1H-pyridin-2-one bis hydrochloride;

(S)-4-[2-(3-Cynao-phenyl)-2-hydroxy-ethylamino]-3-{6[4-(2-methyoxy-ethyl)-piperazin-1-yl]-4-methyl-1H-benzoimidazol-2-yl1H-pyridin-2-one bis hydrochloride;

(S)-4-[2-(3-Chloro-phenyl)-2-hydroxy-ethylamino]-3-{6-[4-(2-hydroxy-ethyl)-piperadin-1-yl]-4-methyl-1H-benzimidazol-2-yl}-1H-pyridin-2-one bis hydrochloride;

(S)-4-[2-(3-Chloro-phenyl)-2-hydroxy-ethylamino]-3-{4-methyl-6-[4-(2-methylsulfanyl-ethyl)-piperazin-1-yl]-1H-benzoimidazol-2-yl}-1H-pyridin-2-one bis hydrochloride;

(S)-4-[2-(3-Chloro-phenyl)-2-hydroxy-ethylamino]-3-[4-methyl-6-(3R-methyl-piperazin-1-yl)-1H-benzoimidazol-2-yl]-1H-pyridin-2-one bis hydrochloride; and

(S)-4-[2-(3-Chloro-phenyl)-2-hydroxy-ethylamino]-3-{6-[4-(2-methoxy-ethyl)-3(R)-methyl-piperazin-1-yl]-4-methyl-1H-benzoimidazol-2-yl}-1H-pyridin-2-one bis hydrochloride.

According the one embodiment of the present invention, IGF1R inhibitors have the following formula II:

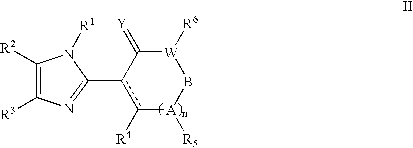

and include its enantiomers, diastereomers, pharmaceutically acceptable salts, hydrates, or esters thereof wherein:

n is 0, 1, 2, or 3;

Y is O or S;

A and B are independently —CH, N, or CO, provided that A and B are not both CO;

W is N, CH, O or S provided that when W is O or S, R⁶ is absent;

R¹, R, and R⁶ are each H or C₁ to C₄ alkyl;

R² is H, C₁₋₆ alkyl, C₂₋₆ alkenyl, C₂₋₆ alkynyl, C₃₋₆ cycloalkyl, heterocycloalkyl, halo, amino, aminoalkyl, alkoxy, thioalkoxy, nitro, aryl, heteroaryl, alkoxyalkyl, thioalkoxyalkyl, aralkyl, heteroarylalkyl, heterocycloalkylalkyl, —CN, —CO₂R⁸, —CONR⁹R¹⁰, —CO₂NR¹¹R¹², —NR¹³CONR¹⁴R¹⁵, —NR¹⁶SO₂R¹⁷, —SO₂NR¹⁸R¹⁹, —C(NR²⁰)NR²¹R²²;

R⁴ and R⁵ are each H, —NH-Z, —NH-Z-aryl, or NH-Z-heteroaryl, wherein

Z is selected from the group consisting of C₁-C₄ alkyl, alkenyl, and alkynyl; Z optionally having one or more hydroxy, thiol, alkoxy, thioalkoxy, amino, halo, NR²³SO₂R²⁴, —CO, —CNOH, —CNOR²⁶, —CNNR²⁷, —CNNCOR²⁸ and —CNNSO₂R²⁹; and

R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, and R²⁶ are independently selected from the group consisting of H, alkyl, alkenyl, alkynyl, cycloalkyl, cycloalkylalkyl, hydroxy, alkoxy, aryl, heteroaryl, heterocyclyl, heteroarylalkyl, and alkyl-R²⁵ wherein R²⁵ is alkenyl, hydroxy, thiol, alkoxy, thioalkoxy, amino, alkylamino, dialkylamino, aryl, heteroaryl, cyano, halo, heteroaryl, heterocyloalkyl, sulfoxy, sulfonyl, —NR²⁷COOR²⁸, —NR²⁹C(O)R³⁰, —NR³¹SO₂R³², SO₂NR³¹R³²—C(O)NR³³R³⁴, and

R²⁷, R²⁸, R²⁹, R³⁰, R³¹, R³², R³³ and R³⁴ are, independently, hydrogen, alkyl, or cycloalkyl.

The IGF1R inhibitors of the present invention are useful in various pharmaceutically acceptable salt forms. The term “pharmaceutically acceptable salt” refers to those salt forms which would be apparent to the pharmaceutical chemist, i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients or their pharmaceutically acceptable salts in combination with pharmaceutically acceptable carriers.

According to the methods of the present invention, IGF1R inhibitors are administered in combination with at least one additional anticancer agent, resulting in a synergistic effect. Additional anticancer agents that are useful in the present invention include, among others, 17α-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, chlorotrianisene, Hydroxyprogesterone, Aminoglutethimide, Estramustine, Medroxyprogesteroneacetate, Leuprolide, Flutamide, Toremifene, Zoladex, matrix metalloproteinase inhibitors, VEGF inhibitors, including as anti-VEGF antibodies such as Avastin, and small molecules such as ZD6474 and SU6668, vatalanib, BAY-43-9006, SU11248, CP-547632, and CEP-7055 are also included. Anti-Her2 antibodies from Genentech (such as Herceptin) may also be utilized. Suitable EGFR inhibitors include gefitinib, erlotinib, and cetuximab. Pan Her inhibitors include canertinib, EKB-569, and GW-572016. Also included are Src inhibitors as well as Casodex® bicalutamide, Astra Zeneca), Tamoxifen, MEK-1 kinase inhibitors, MAPK kinase inhibitors, PI3 inhibitors, and PDGF inhibitors, such as imatinib. Also included are anti-angiogenic and antivascular agents which, by interrupting blood flow to solid tumors, render cancer cells quiescent by depriving them of nutrition. Castration, which also renders androgen dependent carcinomas non-proliferative, may also be utilized. Also included are MET kinase inhibitors, inhibitors of non-receptor and receptor tyrosine kinases, and inhibitors of integrin signaling.

According to one embodiment, the anticancer agent is selected from the group consisting of kinase inhibitors, including a HER-1 inhibitor, HER-2 inhibitor, a HER-4 inhibitor, a pan HER inhibitor, a VEGF inhibitor, a Src inhibitor, a MEK inhibitor, a PDGF inhibitor or a MET inhibitor.

In a preferred embodiment, the anticancer agent is a pan HER inhibitor, an EGFR inhibitor, a MEK inhibitor or a Src inhibitor.

According to one aspect of the present invention, the anticancer agent is an EGFR inhibitor, including small molecule and biological inhibitors, such as, for example, EGFR antibodies and functional equivalents thereof. Functional equivalents of antibodies have binding characteristics comparable to those of antibodies, and inhibit the growth of cells that express EGFR. In some embodiments, the EGFR inhibitor is cetuximab. In another embodiment of the present invention, the EGFR inibitor is erlotinib. In another embodiment of the present invention, the EGFR inhibitor is gefinitib. In another embodiment of the present invention, the EGFR inhibitor is ABX-EGF (Abgenix). In yet another embodiment, the EGFR inhibitor is EMD72000 (Merck KGaA)

EGFR inhibitors that are small molecules and are useful in the present invention include, for example, the following:

U.S. Pat. No. 5,656,655 to Spada et al. discloses styryl substituted heteroaryl compounds that inhibit EGFR. The heteroaryl group is a monocyclic ring with one or two heteroatoms, or a bicyclic ring with 1 to about 4 heteroatoms, the compound being optionally substituted or polysubstituted. The compounds disclosed in U.S. Pat. No. 5,656,655 are incorporated herein by reference.

U.S. Pat. No. 5,646,153 to Spada et al. discloses bis mono and/or bicyclic aryl heteroaryl, carbocyclic, and heterocarbocyclic compounds that inhibit EGFR. The compounds disclosed in U.S. Pat. No. 5,646,153 are incorporated herein by reference.

U.S. Pat. No. 5,679,683 to Bridges et al. discloses tricyclic pyrimidine compounds that inhibit the EGFR. The compounds are fused heterocyclic pyrimidine derivatives described at column 3, line 35 to column 5, line 6. The description of these compounds at column 3, line 35 to column 5, line 6 is incorporated herein by reference.

U.S. Pat. No. 5,616,582 to Barker discloses quinazoline derivatives that have receptor kinase inhibitory activity. The compounds disclosed in U.S. Pat. No. 5,616,582 are incorporated herein by reference.

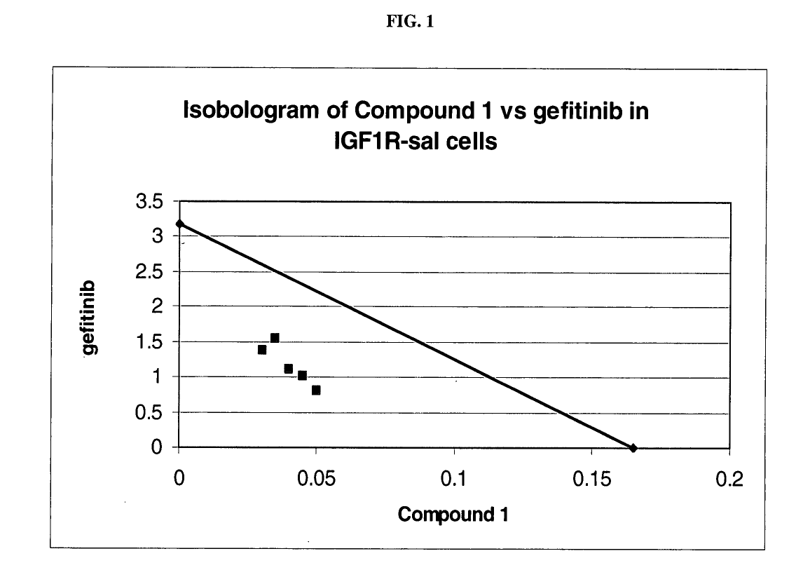

Fry et al., Science 265, 1093-1095 (1994) in FIG. 1 discloses a compound having a structure that inhibits EGFR. The compound shown in FIG. 1 of the Fry et al. article is incorporated herein by reference.

Osherov et al. disclose tyrphostins that inhibit EGFR/HER1. The compounds disclosed in the Osherov et al. article, and, in particular, those in Tables I, II, m, and IV are incorporated herein by reference.

U.S. Pat. No. 5,196,446 to Levitzki et al. discloses heteroarylethenediyl or heteroarylethendeiylaryl compounds that inhibit EGFR. The compounds disclosed in U.S. Pat. No. 5,196,446 from column 2, line 42 to column 3, line 40 are incorporated herein by reference.

Panek et al., Journal of Pharmacology and Experimental Therapeutics 283, hereby incorporated by reference 1433-1444 (1997) discloses a compound identified as PD166285 that inhibits the EGFR, PDGFR, and FGFR families of receptors. PD166285 is identified as 6-(2,6-dichlorophenyl)-2-(4-(2-diethylaminoethyoxy)phenylamino)-8-methyl-8H-pyrido(2,3-d)pyrimidin-7-one having the structure shown in FIG. 1 on page 1436.

According to another aspect of the present invention, the anticancer agent is a a Src inhibitor. Src inhibitors are described in U.S. patent application Ser. No. 10/378,373, filed Mar. 3, 2003, the disclosure of which is herein incorporated by reference in its entirety.

According to one preferred embodiment, the Src inhibitor has the formula III:

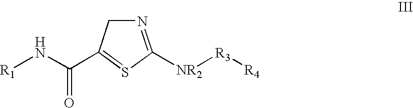

wherein:

each R₁, R₃ and R₄ is, independently, a heterocyclic group or an aryl group, optionally substituted with one or more substituents; and

R₂ is hydrogen or alkyl.

Src compounds that are useful in the present invention include, among others, those selected from the following list:

[5-[[(2,4,6-Trimethylphenyl)amino]carbonyl]-4-methyl-2-thiazolyl]carbamic acid, 1,1-dimethylethyl ester;

[5-[[(2,4,6-Trimethylphenyl)amino]carbonyl]-4-trifluoromethyl-2-thiazolyl]carbamic acid, 1,1-dimethylethyl ester;

2-Amino-N-(2,4,6-trimethylphenyl)-4-trifluoromethyl-5-thiazolecarboxamide, trifluoroacetate (1:1);

[5-[[(2,4,6-Trimethylphenyl)amino]carbonyl]-4-phenyl-2-thiazolyl]carbamic acid, 1,1-dimethylethyl ester;

2-Amino-N-(2,4,6-trimethylphenyl)-4-phenyl-5-thiazolecarboxamide, trifluoroacetate (1:1);

[5-[[phenylamino]carbonyl]-4-methyl-2-thiazolyl]carbamic acid, 1,1-dimethylethyl ester;

[5-[[(2,4-Dichlorophenyl)amino]carbonyl]-4-methyl-2-thiazolyl]carbamic acid, 1,1-dimethylethyl ester;

5-[[(2,4,6-Trimethylphenyl)amino]carbonyl]-2-thiazolyl]carbamic acid, 1,1-dimethylethyl ester;

2-Amino-N-(2,4,6-trimethylphenyl)-4-phenyl-5-thiazolecarboxamide, trifluoroacetate (1:1);

[5-[[(2-Methoxy-6-methylphenyl)amino]carbonyl]-4-methyl-2-thiazolyl]carbamic acid 1,1-dimethylethyl ester;

[4-Methyl-5-[[[3-methyl-4-(1-methylethyl)phenyl]amino]carbonyl]-2-thiazolyl]carbamic acid 1,1-dimethylethyl ester;

[5-[[(4-Bromo-2,6-dimethylphenyl)amino]carbonyl]-4-methyl-2-thiazolyl]carbamic acid 1,1-dimethylethyl ester;

[4-Methyl-5-[[[2-methyl-6-(1-methylethyl)phenyl]amino]carbonyl]-2-thiazolyl]carbamic acid 1,1-dimethylethyl ester;

[5-[[(2,4-Dimethylphenyl)amino]carbonyl]-4-methyl-2-thiazolyl]carbamic acid 1,1-dimethylethyl ester;

[4-Methyl-5-[[(2-methylphenyl)amino]carbonyl]-2-thiazolyl]carbamic acid 1,1-dimethylethyl ester;

[5-[[(2-Chloro-6-methylphenyl)amino]carbonyl]-4-methyl-2-thiazolyl]carbamic acid 1,1-dimethylethyl ester;

[5-[[[2-(1,1-Dimethylethyl)-4-methylphenyl]amino]carbonyl]-4-methyl-2-thiazolyl]carbamic acid 1,1-dimethylethyl ester;

[5-[[(2-Furanylmethyl)amino]carbonyl]-4-methyl-2-thiazolyl]carbamic acid 1,1-dimethylethyl ester;

[5-[[[3-Methoxy-5-(trifluoromethyl)phenyl]amino]carbonyl]-4-methyl-2-thiazolyl]carbamic acid 1,1-dimethylethyl ester;

[5-[[(4-Cyclohexylphenyl)amino]carbonyl]-4-methyl-2-thiazolyl]carbamic acid 1,1-dimethylethyl ester;

[5-[[(Cyclohexylmethyl)amino]carbonyl]-4-methyl-2-thiazolyl]carbamic acid 1,1-dimethylethyl ester;

[5-[[(2,3-Dihydro-1H-indenyl)amino]carbonyl]-4-methyl-2-thiazolyl]carbamic acid 1,1-dimethylethyl ester;

[5-[(2,5-Dihydro-1H-pyrrol-1-yl)carbonyl]-4-methyl-2-thiazolyl]carbamic acid 1,1-dimethylethyl ester;

[5-[(2,5-Dihydro-2,5-dimethyl-1H-pyrrol-1-yl)carbonyl]-4-methyl-2-thiazolyl]carbamic acid 1,1-dimethylethyl ester;

1-[[2-[[(1,1-Dimethylethoxy)carbonyl]amino]-4-methyl-5-thiazolyl]carbonyl]-L-prolinamide;

[5-[(4-Formyl-1-piperazinyl)carbonyl]-4-methyl-2-thiazolyl]carbamic acid 1,1-dimethylethyl ester;

[5-(1,4-Dioxa-8-azaspiro[4.5]decan-8-ylcarbonyl)-4-methyl-2-thiazolyl]carbamic acid 1,1-dimethylethyl ester;

[5-[[3-[(Diethylamino)carbonyl]-1-piperidinyl]carbonyl]-4-methyl-2-thiazolyl]carbamic acid 1,1-dimethylethyl ester;

[4-Methyl-5-[(octahydro-1-quinolinyl)carbonyl]-2-thiazolyl]carbamic acid 1,1-dimethylethyl ester;

2-[[(1,1-Dimethylethoxy)carbonyll]amino]-4-methyl-5-thiazolecarboxylic acid 2-[(1,1-dimethylethoxy)carbonyl]hydrazide;

[5-[[(4-Methoxyphenyl)amino]carbonyl]-4-methyl-2-thiazolyl]carbamic acid 1,1-dimethylethyl ester;

[4-Methyl-5-[[(4-methylphenyl)amino]carbonyl]-2-thiazolyl]carbamic acid 1,1-dimethylethyl ester;

[5-[[(1,2-Dimethylpropyl)amino]carbonyl]-4-methyl-2-thiazolyl]carbamic acid 1,1-dimethylethyl ester;

[5-[[(2,2-Dimethylpropyl)amino]carbonyl]-4-methyl-2-thiazolyl]carbamic acid 1,1-dimethylethyl ester;

[4-Methyl-5-[(2-propynylamino)carbonyl]-2-thiazolyl]carbamic acid 1,1-dimethylethyl ester;

[4-Methyl-5-[(2-propenylamino)carbonyl]-2-thiazolyl]carbamic acid 1,1-dimethylethyl ester;

[4-Methyl-5-[(methylphenylamino)carbonyl]-2-thiazolyl]carbamic acid 1,1-dimethylethyl ester;

[4-Methyl-5-[[(3,4,5-trimethoxyphenyl)amino]carbonyl]-2-thiazolyl]carbamic acid 1,1-dimethylethyl ester;

[5-[[[2,6-Bis(1-methylethyl)phenyl]amino]carbonyl]-4-methyl-2-thiazolyl]carbamic acid 1,1-dimethylethyl ester;

[5-[[[3-(1H-Imidazol-1-yl)propyl]amino]carbonyl]-4-methyl-2-thiazolyl]carbamic acid 1,1-dimethylethyl ester;

[5-[[[(3,4-Difluorophenyl)methyl]amino]carbonyl]-4-methyl-2-thiazolyl]carbamic acid 1,1-dimethylethyl ester;

N-[[2-[[(1,1-Dimethylethoxy)carbonyl]amino]-4-methyl-5-thiazolyl]carbonyl]-L-leucine methyl ester;

5-[[[2-[[(1,1-Dimethylethoxy)carbonyl]amino]-4-methyl-5-thiazolyl]carbonyl]amino]-4-oxopentanoic acid methyl ester;

[5-[[[2-(Ethylthio)ethyl]amino]carbonyl]-4-methyl-2-thiazolyl]carbamic acid 1,1-dimethylethyl ester;

[5-[[Bis(3-methylbutyl)amino]carbonyl]-4-methyl-2-thiazolyl]carbamic acid 1,1-dimethylethyl ester;

[5-[[Ethyl(1-methylethyl)amino]carbonyl]-4-methyl-2-thiazolyl]carbamic acid 1,1-dimethylethyl ester;

2-[[(1,1-Dimethylethoxy)carbonyl]amino]-4-methyl-5-thiazolecarboxylic acid 2-[[(3,5-dichlorophenyl)amino]thioxomethyl]hydrazide;

[5-[[Bis(2-ethoxyethyl)amino]carbonyl]-4-methyl-2-thiazolyl]carbamic acid 1,1-dimethylethyl ester;

[4-Methyl-5-[[3-[(trifluoroacetyl)amino]-1-pyrrolidinyl]carbonyl]-2-thiazolyl]carbamic acid 1,1-dimethylethyl ester;

[5-[[(2,6-Dimethylphenyl)amino]carbonyl]-4-methyl-2-thiazolyl]carbamic acid 1,1-dimethylethyl ester;

2-[[(2,2-Dichloro-1-methylcyclopropyl)carbonyl]amino]-4-methyl-N-(2,4,6-trimethylphenyl)-5-thiazolecarboxamide;

2-[(Cyclohexylacetyl)amino]-4-methyl-N-(2,4,6-trimethylphenyl)-5-thiazolecarboxamide;

2-[(2,5-Difluorobenzoyl)amino]-4-methyl-N-(2,4,6-trimethylphenyl)-5-thiazolecarboxamide;

2-[(5-Bromo-2-chlorobenzoyl)amino]-4-methyl-N-(2,4,6-trimethylphenyl)-5-thiazolecarboxamide;

2-[(3-Cyanobenzoyl)amino]-4-methyl-N-(2,4,6-trimethylphenyl)-5-thiazolecarboxamide;

2-[[4-(Acetylamino)benzoyl]amino]-4-methyl-N-(2,4,6-trimethylphenyl)-5-thiazolecarboxamide;

4-Methyl-2-[[3-(trifluoromethyl)benzoyl]amino]-N-(2,4,6-trimethylphenyl)-5-thiazolecarboxamide;

4-Methyl-2-[[2-(2-phenylethyl)benzoyl]amino]-N-(2,4,6-trimethylphenyl)-5-thiazolecarboxamide;

2-[(3,5-Dimethylbenzoyl)amino]-4-methyl-N-(2,4,6-trimethylphenyl)-5-thiazolecarboxamide;

2-[(4-Ethenylbenzoyl)amino]-4-methyl-N-(2,4,6-trimethylphenyl)-5-thiazolecarboxamide;

2-[(4-Butylbenzoyl)amino]-4-methyl-N-(2,4,6-trimethylphenyl)-5-thiazolecarboxamide;

4-Methyl-2-[(4-pentylbenzoyl)amino]-N-(2,4,6-trimethylphenyl)-5-thiazolecarboxamide;

4-Methyl-2-[(1-oxo-3-phenoxypropyl)amino]-N-(2,4,6-trimethylphenyl)-5-thiazolecarboxamide;

4-Methyl-2-[(1-oxo-3-phenylpropyl)amino]-N-(2,4,6-trimethylphenyl)-5-thiazolecarboxamide;

2-[[3-(2-Methoxyphenyl)-1-oxopropyl]amino]-4-methyl-N-(2,4,6-trimethylphenyl)-5-thiazolecarboxamide;

4-Methyl-2-[(2-naphthalenylacetyl)amino]-N-(2,4,6-trimethylphenyl)-5-thiazolecarboxamide;

2-[(Diphenylacetyl)amino]-4-methyl-N-(2,4,6-trimethylphenyl)-5-thiazolecarboxamide;

2-[[(2-Chloro-6-fluorophenyl)acetyl]amino]-4-methyl-N-(2,4,6-trimethylphenyl)-5-thiazolecarboxamide;

4-Methyl-2-[[(2-methylphenyl)acetyl]amino]-N-(2,4,6-trimethylphenyl)-5-thiazolecarboxamide;

2-[[(3-Methoxyphenyl)acetyl]amino]-4-methyl-N-(2,4,6-trimethylphenyl)-5-thiazolecarboxamide;

2-[[(3,4-Dimethoxyphenyl)acetyl]amino]-4-methyl-N-(2,4,6-trimethylphenyl)-5-thiazolecarboxamide;

2-[[(4-Chlorophenyl)acetyl]amino]-4-methyl-N-(2,4,6-trimethylphenyl)-5-thiazolecarboxamide;

2-[([1,1′-Biphenyl]-4-ylacetyl)amino]-4-methyl-N-(2,4,6-trimethylphenyl)-5-thiazolecarboxamide;

4-Methyl-2-[(1-oxo-4-phenylbutyl)amino]-N-(2,4,6-trimethylphenyl)-5-thiazolecarboxamide;

2-[(2-Hydroxy-2-phenyl-1-oxopropyl)amino]-4-methyl-N-(2,4,6-trimethylphenyl)-5-thiazolecarboxamide;

2-[(2-Hydroxy-1-oxohexyl)amino]-4-methyl-N-(2,4,6-trimethylphenyl)-5-thiazolecarboxamide;

4-Methyl-2-[[1-oxo-4-(2-thienyl)butyl]amino]-N-(2,4,6-trimethylphenyl)-5-thiazolecarboxamide;

4-Methyl-2-[(3-thienylcarbonyl)amino]-N-(2,4,6-trimethylphenyl)-5-thiazolecarboxamide;

2-[(2-Benzofuranylcarbonyl)amino]-4-methyl-N-(2,4,6-trimethylphenyl)-5-thiazolecarboxamide;

N-[4-Methyl-5-[[(2,4,6-trimethylphenyl)amino]carbonyl]-2-thiazolyl]-4-pyridinecarboxamide, N-oxide;

6-Chloro-N-[4-methyl-5-[[(2,4,6-trimethylphenyl)amino]carbonyl]-2-thiazolyl]-3-pyridinecarboxamide;

N-[4-Methyl-5-[[(2,4,6-trimethylphenyl)amino]carbonyl]-2-thiazolyl]-3-pyridinecarboxamide;

N-[4-Methyl-5-[[(2,4,6-trimethylphenyl)amino]carbonyl]-2-thiazolyl]-3-quinolinecarboxamide;

4-Methyl-2-[[(4-nitrophenyl)acetyl]amino]-N-(2,4,6-trimethylphenyl)-5-thiazolecarboxamide;

4-Methyl-2-[(2,4,6-trichlorobenzoyl)amino]-N-(2,4,6-trimethylphenyl)-5-thiazolecarboxamide;

4-Methyl-2-[[2-[[3-(trifluoromethyl)phenyl]amino]benzoyl]amino]-N-(2,4,6-trimethylphenyl)-5-thiazolecarboxamide;

4-Methyl-2-[[4-(4-nitrophenyl)-1-oxobutyl]amino]-N-(2,4,6-trimethylphenyl)-5-thiazolecarboxamide;

4-Methyl-2-[[4-(methylsulfonyl)benzoyl]amino]-N-(2,4,6-trimethylphenyl)-5-thiazolecarboxamide;

2-[(4-Heptylbenzoyl)amino]-4-methyl-N-(2,4,6-trimethylphenyl)-5-thiazolecarboxamide;

2-[[(2,4-Difluorophenyl)acetyl]amino]-4-methyl-N-(2,4,6-trimethylphenyl)-5-thiazolecarboxamide;

(S)-2-[[2-(Dipropylamino)-1-oxopropyl]amino]-4-methyl-N-(2,4,6-trimethylphenyl)-5-thiazolecarboxamide;

2-[(2-Biphenylenecarbonyl)amino]-4-methyl-N-(2,4,6-trimethylphenyl)-5-thiazolecarboxamide;

2-[[3-(3-Methoxyphenyl)-1-oxopropyl]amino]-4-methyl-N-(2,4,6-trimethylphenyl)-5-thiazolecarboxamide;

4-Methyl-N-(2,4,6-trimethylphenyl)-2-[[(2,4,6-trimethylphenyl)acetyl]amino]-5-thiazolecarboxamide;

4-Methyl-2-[(1-oxo-6-heptenyl)amino]-N-(2,4,6-trimethylphenyl)-5-thiazolecarboxamide;

2-[[(1,3-Benzodioxol-5-yl)acetyl]amino]-4-methyl-N-(2,4,6-trimethylphenyl)-5-thiazolecarboxamide;

4-Methyl-2-[[[2-(phenylmethoxy)phenyl]acetyl]amino]-N-(2,4,6-trimethylphenyl)-5-thiazolecarboxamide;

4-Methyl-2-[[(3-phenoxyphenyl)acetyl]amino]-N-(2,4,6-trimethylphenyl)-5-thiazolecarboxamide;

2-[(3,5-Dimethoxyphenyl)acetyl]amino]-4-methyl-N-(2,4,6-trimethylphenyl)-5-thiazolecarboxamide;

2-[[4-[4-[Bis(2-chloroethyl)amino]phenyl]-1-oxobutyl]amino]-4-methyl-N-(2,4,6-trimethylphenyl)-5-thiazolecarboxamide;

4-[[4-[[[4-methyl-5-[[(2,4,6-trimethylphenyl)amino]carbonyl]-2-thiazolyl]amino]carbonyl]phenyl}amino]-4-oxobutanoic acid methyl ester;

4-Methyl-2-[[(phenylsulfonyl)acetyl]amino]-N-(2,4,6-trimethylphenyl)-5-thiazolecarboxamide;

2-[[2-(Acetylamino)-1-oxohexyl]amino]-4-methyl-N-(2,4,6-trimethylphenyl)-5-thiazolecarboxamide;

2-[[4-[(Dipropylamino)sulfonyl]benzoyl]amino]-4-methyl-N-(2,4,6-trimethylphenyl)-5-thiazolecarboxamide;

2-[(4-Cyclohexylbenzoyl)amino]-4-methyl-N-(2,4,6-trimethylphenyl)-5-thiazolecarboxamide;

2-[(4-Bromo-3-methylbenzoyl)amino]-4-methyl-N-(2,4,6-trimethylphenyl)-5-thiazolecarboxamide;

2-[[(2,3-Difluorophenyl)acetyl]amino]-4-methyl-N-(2,4,6-trimethylphenyl)-5-thiazolecarboxamide;

4-Methyl-2-[[[4-(1-methylethyl)phenyl]acetyl]amino]-N-(2,4,6-trimethylphenyl)-5-thiazolecarboxamide;

2-[[[4-(1,1-Dimethylethyl)cyclohexyl]carbonyl]amino]-4-methyl-N-(2,4,6-trimethylphenyl)-5-thiazolecarboxamide;

N,N-Dimethyl-N′-[4-methyl-5-[[(2,4,6-trimethylphenyl)amino]carbonyl]-2-thiazolyl]butanediamide;

2-[(1,6-Dioxohexyl)amino]-4-methyl-N-(2,4,6-trimethylphenyl)-5-thiazolecarboxamide;

2-[(Benzo[b]thiophen-2-ylcarbonyl)amino]-4-methyl-N-(2,4,6-trimethylphenyl)-5-thiazolecarboxamide;

2-[(1-Adamantylcarbonyl)amino]-4-methyl-N-(2,4,6-trimethylphenyl)-5-thiazolecarboxamide;

4-Methyl-2-[[(4-methylcyclohexyl)carbonyl]amino]-N-(2,4,6-trimethylphenyl)-5-thiazolecarboxamide;

2-[(1,7-Dioxooctyl)amino]-4-methyl-N-(2,4,6-trimethylphenyl)-5-thiazolecarboxamide;

2-[[2-(Acetylamino)-4-(ethylthio)-1-oxobutyl]amino]-4-methyl-N-(2,4,6-trimethylphenyl)-5-thiazolecarboxamide;

1,5-Dimethyl-N-[4-methyl-5-[[(2,4,6-trimethylphenyl)amino]carbonyl]-2-thiazolyl]-1H-pyrazole-3-carboxamide;

2-[[[4-methyl-5-[[(2,4,6-trimethylphenyl)amino]carbonyl]-2-thiazolyl]amino]carbonyl]benzoic acid;

N-[4-Methyl-5-[[(2,4,6-trimethylphenyl)amino]carbonyl]-2-thiazolyl]-6-benzothiazolecarboxamide;

1-Ethyl-4-methyl-N-[4-methyl-5-[[(2,4,6-trimethylphenyl)amino]carbonyl]-2-thiazolyl]-1H-pyrazole-3-carboxamide;

4-Methyl-2-[[3-[(3H-1,2,3-triazolo[4,5-b]pyridin-3-yloxy)methyl]benzoyl]amino]-N-(2,4,6-trimethylphenyl)-5-thiazolecarboxamide;

2-[(2-Furanylcarbonyl)amino]-4-methyl-N-(2,4,6-trimethylphenyl)-5-thiazolecarboxamide;

2-[(4-Chlorobenzoyl)amino]-4-methyl-N-(2,4,6-trimethylphenyl)-5-thiazolecarboxamide;

[4-Methyl-5[[(2-nitrophenyl)amino]carbonyl]-2-thiazolyl]carbamic acid, 1,1-dimethylethyl ester;

[4-Methyl-5 [[(2,4,6-trimethylphenyl)amino]carbonyl]-2-thiazolyl]carbamic acid, phenylmethyl ester;

Methyl[4-methyl-5-[[(2,4,6-trimethylphenyl)amino]carbonyl]-2-thiazolyl]carbamic acid, 1,1-dimethylethyl ester;

[4-Methyl-5-[[methyl(2,4,6-trimethylphenyl)amino]carbonyl]-2-thiazolyl]carbamic acid, 1,1-dimethylethyl ester;

[4-Methyl-5 [[(2,4,6-trimethylphenyl)amino]carbonyl]-2-thiazolyl]carbamic acid, methyl ester;

[4-Ethyl-5 [[(2,4,6-trimethylphenyl)amino]carbonyl]-2-thiazolyl]carbamic acid, 1,1-dimethylethyl ester;

[5-[[(2,6-Dichlorophenyl)amino]carbonyl]-4-methyl-2-thiazolyl]carbamic acid, 1,1-dimethylethyl ester;

2-Amino-N-(2-methyl-6-isopropylphenyl)-4-methyl-5-thiazolecarboxamide, trifluoroacetate (1:1);

2-(Benzoylamino)-4-methyl-N-(2,4,6-trimethylphenyl)-5-thiazolecarboxamide;

4-Methyl-2-[(phenylcetyl)amino]-N-(2,4,6-trimethylphenyl)-5-thiazolecarboxamide;

2-[[(Acetylamino)acetyl]amino]-4-methyl-N-(2,4,6-trimethylphenyl)-6-thiazolecarboxamide;

2-Amino-4-methyl-N-(2,4,6-trimethylphenyl)-5-thiazolecarbothioamide;

2-[(4-Bromobenzoyl)amino]-4-methyl-N-(2,4,6-trimethylphenyl)-5-thiazolecarboxamide;

4-Methyl-2-[(4-nitrobenzoyl)amino]-N-(2,4,6-trimethylphenyl)-5-thiazolecarboxamide;

2-[(4-Cyanobenzoyl)amino]-4-methyl-N-(2,4,6-trimethylphenyl)-5-thiazolecarboxamide;

4-Methyl-2-[[(5-nitro-2-furanyl)carbonyl]amino]-N-(2,4,6-trimethylphenyl)-5-thiazolecarboxamide;

4-Methyl-2-[(2-thienylcarbonyl)amino]-N-(2,4,6-trimethylphenyl)-5-thiazolecarboxamide;

4-[[[4-Methyl-5-[[(2,4,6-trimethylphenyl)amino]carbonyl]-2-thiazolyl]amino]carbonyl]benzoic acid methyl ester;

2-[(5-Isoxazolylcarbonyl)amino]-4-methyl-N-(2,4,6-trimethylphenyl)-5-thiazolecarboxamide;

2-[(3-Furanylcarbonyl)amino]-4-methyl-N-(2,4,6-trimethylphenyl)-5-thiazolecarboxamide;

2-[[(2,4-Dimethyl-5-thiazolyl)carbonyl]amino]-4-methyl-N-(2,4,6-trimethylphenyl)-5-thiazolecarboxamide;

2-[[(4-Methoxy-3-thienyl)carbonyl]amino]-4-methyl-N-(2,4,6-trimethylphenyl)-5-thiazolecarboxamide;

4-Methyl-2-[[(5-nitro-3-thienyl)carbonyl]amino]-N-(2,4,6-trimethylphenyl)-5-thiazolecarboxamide;

2-[[[4-[(4-Chlorophenyl)thio]-3-thienyl]carbonyl]amino]-4-methyl-N-(2,4,6-trimethylphenyl)-5-thiazolecarboxamide;

2-[[(5-Chloro-4-methoxy-3-thienyl)carbonyl]amino]-4-methyl-N-(2,4,6-trimethylphenyl)-5-thiazolecarboxamide;

2-[[[2-(4,5-Dihydro-4,4-dimethyl-2-oxazolyl)-3-thienyl]carbonyl]amino]-4-methyl-N-(2,4,6-trimethylphenyl)-5-thiazolecarboxamide;

2-[[(2-Acetyl-3-thienyl)carbonyl]amino]-4-methyl-N-(2,4,6-trimethylphenyl)-5-thiazolecarboxamide;

4-Methyl-2-[[(methylamino)carbonyl]amino]-N-(2,4,6-trimethylphenyl)-5-thiazolecarboxamide;

4-Methyl-2-[[(phenylamino)carbonyl]amino]-N-(2,4,6-trimethylphenyl)-5-thiazolecarboxamide;

4-Methyl-2-[[[(4-methylphenyl)amino]carbonyl]amino]-N-(2,4,6-trimethylphenyl)-5-thiazolecarboxamide;

4-Methyl-2-[[[(phenylmethyl)amino]carbonyl]amino]-N-(2,4,6-trimethylphenyl)-5-thiazolecarboxamide;

2-[[(Butylamino)carbonyl]amino]-4-methyl-N-(2,4,6-trimethylphenyl)-5-thiazolecarboxamide;

4-Methyl-2-[[(propylamino)carbonyl]amino]-N-(2,4,6-trimethylphenyl)-5-thiazolecarboxamide;

2-[[(Cyclohexylamino)carbonyl]amino]-4-methyl-N-(2,4,6-trimethylphenyl)-5-thiazolecarboxamide;

2-[[[(2-Chlorophenyl)amino]carbonyl]amino]-4-methyl-N-(2,4,6-trimethylphenyl)-5-thiazolecarboxamide;

2-[[[(3-Fluorophenyl)amino]carbonyl]amino]-4-methyl-N-(2,4,6-trimethylphenyl)-5-thiazolecarboxamide;

2-[[[(2,6-Dimethylphenyl)amino]carbonyl]amino]-4-methyl-N-(2,4,6-trimethylphenyl)-5-thiazolecarboxamide;

[5-[[(2,4,6-Trimethylphenyl)amino]carbonyl]-4-methyl-2-thiazolyl]carbamic acid, phenyl ester;

4-Methyl-2-[[[(2-phenylethyl)amino]carbonyl]amino]-N-(2,4,6-trimethylphenyl)-5-thiazolecarboxamide

2-[[(Hexylamino)carbonyl]amino]-4-methyl-N-(2,4,6-trimethylphenyl)-5-thiazolecarboxamide;

2-[[[(1,1-Dimethylethyl)amino]carbonyl]amino]-4-methyl-N-(2,4,6-trimethylphenyl)-5-thiazolecarboxamide;

2-[[[(3-Fluoro-4-methylphenyl)amino]carbonyl]amino]-4-methyl-N-(2,4,6-trimethylphenyl)-5-thiazolecarboxamide;

2-[[[(4-Methoxyphenyl)amino]carbonyl]amino]-4-methyl-N-(2,4,6-trimethylphenyl)-5-thiazolecarboxamide;

2-[[(Diethylamino)carbonyl]amino]-4-methyl —N-(2,4,6-trimethylphenyl)-5-thiazolecarboxamide;

2-[[[Bis(1-methylethyl)amino]carbonyl]amino]-4-methyl-N-(2,4,6-trimethylphenyl)-5-thiazolecarboxamide;

4-Methyl-2-[[[methyl(phenylmethyl)amino]carbonyl]amino]-N-(2,4,6-trimethylphenyl)-5-thiazolecarboxamide;

4-Methyl-2-[[(methylphenylamino)carbonyl]amino]-N-(2,4,6-trimethylphenyl)-5-thiazolecarboxamide;

2-[[(Cyclohexylmethylamino)carbonyl]amino]-4-methyl-N-(2,4,6-trimethylphenyl)-5-thiazolecarboxamide;

4-Methyl-2-[[[(1-phenylethyl)amino]carbonyl]amino]-N-(2,4,6-trimethylphenyl)-5-thiazolecarboxamide;

2-[[[(Cyclopropylmethyl)propylamino]carbonyl]amino]-4-methyl-N-(2,4,6-trimethylphenyl)-5-thiazolecarboxamide;

4-Methyl-2-[[[(2-methylcyclohexyl)amino]carbonyl]amino]-N-(2,4,6-trimethylphenyl)-5-thiazolecarboxamide;

4-Methyl-2-[[[(4-methylcyclohexyl)amino]carbonyl]amino]-N-(2,4,6-trimethylphenyl)-5-thiazolecarboxamide;

2-[[[(Cyclohexylmethyl)amino]carbonyl]amino]-4-methyl-N-(2,4,6-trimethylphenyl)-5-thiazolecarboxamide;

2-[[[(2,3-Dihydro-1H-inden-1-yl)amino]carbonyl]amino]-4-methyl-N-(2,4,6-trimethylphenyl)-5-thiazolecarboxamide;

4-Methyl-2-[[[(1-naphthalenylmethyl)amino]carbonyl]amino]-N-(2,4,6-trimethylphenyl)-5-thiazolecarboxamide

2-[[[Bis(phenylmethyl)amino]carbonyl]amino]-4-methyl-N-(2,4,6-trimethylphenyl)-5-thiazolecarboxamide;

2,6-Dimethyl-N-[4-methyl-5-[[(2,4,6-trimethylphenyl)amino]carbonyl]-2-thiazolyl]-4-morpholinecarboxamide;

2-Ethyl-N-[4-methyl-5-[[(2,4,6-trimethylphenyl)amino]carbonyl]-2-thiazolyl]-1-piperidinecarboxamide;

1-[[[4-Methyl-5-[[(2,4,6-trimethylphenyl)amino]carbonyl]-2-thiazolyl]amino]carbonyl]-3-piperidinecarboxylic acid ethyl ester;

3,3-Dimethyl-N-[4-methyl-5-[[(2,4,6-trimethylphenyl)amino]carbonyl]-2-thiazolyl]-1-piperidinecarboxamide

1-[[[4-Methyl-5-[[(2,4,6-trimethylphenyl)amino]carbonyl]-2-thiazolyl]amino]carbonyl]-4-piperidinecarboxylic acid ethyl ester;

4-Methyl-2-[[[(3-methyl-2-pyridinyl)amino]carbonyl]amino]-N-(2,4,6-trimethylphenyl)-5-thiazolecarboxamide

4-Methyl-2-[[[1-(phenylmethyl)-4-piperidinyl]amino]carbonyl]amino]-N-(2,4,6-trimethylphenyl)-5-thiazolecarboxamide;

Octahydro-N-[4-methyl-5-[[(2,4,6-trimethylphenyl)amino]carbonyl]-2-thiazolyl]-1 (2H)-quinolinecarboxamide;

3,4-Dihydro-N-[4-methyl-5-[[(2,4,6-trimethylphenyl)amino]carbonyl]-2-thiazolyl]-2(1H)-isoquinolinecarboxamide;

2-[[[(1,5-Dimethylhexyl)amino]carbonyl]amino]-4-methyl-N-(2,4,6-trimethylphenyl)-5-thiazolecarboxamide;

4-Methyl-2-[[[(1-methylheptyl)amino]carbonyl]amino]-N-(2,4,6-trimethylphenyl)-5-thiazolecarboxamide;

2-[[[[(2-Fluorophenyl)methyl]amino]carbonyl]amino]-4-methyl-N-(2,4,6-trimethylphenyl)-5-thiazolecarboxamide;

2-[[[[(2-Methoxyphenyl)methyl]amino]carbonyl]amino]-4-methyl-N-(2,4,6-trimethylphenyl)-5-thiazolecarboxamide;

2-[[[[(2-Ethoxyphenyl)methyl]amino]carbonyl]amino]-4-methyl-N-(2,4,6-trimethylphenyl)-5-thiazolecarboxamide;

2-[[[[(3-Methoxyphenyl)methyl]amino]carbonyl]amino]-4-methyl-N-(2,4,6-trimethylphenyl)-5-thiazolecarboxamide;

2-[[[[(4-Chlorophenyl)methyl]amino]carbonyl]amino]-4-methyl-N-(2,4,6-trimethylphenyl)-5-thiazolecarboxamide;

2-[[[[(4-Methoxyphenyl)methyl]amino]carbonyl]amino]-4-methyl-N-(2,4,6-trimethylphenyl)-5-thiazolecarboxamide;

2-[[[(2,2-Diphenylethyl)amino]carbonyl]amino]-4-methyl-N-(2,4,6-trimethylphenyl)-5-thiazolecarboxamide

2-[[[(2-Aminoethyl)phenylamino]carbonyl]amino]-4-methyl-N-(2,4,6-trimethylphenyl)-5-thiazolecarboxamide;

2-[[[[2-(3-Methoxyphenyl)ethyl]amino]carbonyl]amino]-4-methyl-N-(2,4,6-trimethylphenyl)-5-thiazolecarboxamide;

2-[[[[2-(3,4-Dimethoxyphenyl)ethyl]amino]carbonyl]amino]-4-methyl-N-(2,4,6-trimethylphenyl)-5-thiazolecarboxamide;

2-[[[[2-(4-Methoxyphenyl)ethyl]amino]carbonyl]amino]-4-methyl-N-(2,4,6-trimethylphenyl)-5-thiazolecarboxamide;

4-Methyl-2-[[[(3-phenylpropyl)amino]carbonyl]amino]-N-(2,4,6-trimethylphenyl)-5-thiazolecarboxamide;

2-[[[[2-(Cyclohex-1-en-1-yl)ethyl]amino]carbonyl]amino]-4-methyl-N-(2,4,6-trimethylphenyl)-5-thiazolecarboxamide;

2-[[[[4-(1,1-Dimethylethyl)cyclohexyl]amino]carbonyl]amino]-4-methyl-N-(2,4,6-trimethylphenyl)-5-thiazolecarboxamide;

2-[[[(3-Butoxypropyl)amino]carbonyl]amino]-4-methyl-N-(2,4,6-trimethylphenyl)-5-thiazolecarboxamide;

2-[[[[2-(2-Methoxyphenyl)ethyl]amino]carbonyl]amino]-4-methyl-N-(2,4,6-trimethylphenyl)-5-thiazolecarboxamide;

2-[[[[(2-Chloro-4-fluorophenyl)methyl]amino]carbonyl]amino]-4-methyl-N-(2,4,6-trimethylphenyl)-5-thiazolecarboxamide;

2-[[(Hexylmethylamino)carbonyl]amino]-4-methyl-N-(2,4,6-trimethylphenyl)-5-thiazolecarboxamide;

2-[[[[1-(4-Chlorophenyl)ethyl]amino]carbonyl]amino]-4-methyl-N-(2,4,6-trimethylphenyl)-5-thiazolecarboxamide;

2-[[[[2-(3-Chlorophenyl)ethyl]amino]carbonyl]amino]-4-methyl-N-(2,4,6-trimethylphenyl)-5-thiazolecarboxamide;

4-Methyl-2-[[[[2-(2-thienyl)ethyl]amino]carbonyl]amino]-N-(2,4,6-trimethylphenyl)-5-thiazolecarboxamide;

2-[[[[2-(2-Fluorophenyl)ethyl]amino]carbonyl]amino]-4-methyl-N-(2,4,6-trimethylphenyl)-5-thiazolecarboxamide;

4-Methyl-2-[[[[2-(2-pyridinyloxy)ethyl]amino]carbonyl]amino]-N-(2,4,6-trimethylphenyl)-5-thiazolecarboxamide;

2-[[[[(2-Bromo-4,5-dimethoxyphenyl)methyl]methylamino]carbonyl]amino]-4-methyl-N-(2,4,6-trimethylphenyl)-5-thiazolecarboxamide;

(E)-2-[[[(3,7-Dimethyl-2,6-octadienyl)amino]carbonyl]amino]-4-methyl-N-(2,4,6-trimethylphenyl)-5-thiazolecarboxamide;

2-[[[[(2,3-Dihydro-1,4-benzodioxin-2-yl)methyl]amino]carbonyl]amino]-4-methyl-N-(2,4,6-trimethylphenyl)-5-thiazolecarboxamide;

2-[[[[3-Methoxy-5-(trifluoromethyl)phenyl]amino]carbonyl]amino]-4-methyl-N-(2,4,6-trimethylphenyl)-5-thiazolecarboxamide;

2-[[[(4-Cyclohexylphenyl)amino]carbonyl]amino]-4-methyl-N-(2,4,6-trimethylphenyl)-5-thiazolecarboxamide;

4-Methyl-2-[[[(5,6,7,8-tetrahydro-1-naphthalenyl)amino]carbonyl]amino]-N-(2,4,6-trimethylphenyl)-5-thiazolecarboxamide;

2-[[(1-Anthracenylamino)carbonyl]amino]-4-methyl-N-(2,4,6-trimethylphenyl)-5-thiazolecarboxamide;

2-[[[(4-Chloro-1-naphthalenyl)amino]carbonyl]amino]-4-methyl-N-(2,4,6-trimethylphenyl)-5-thiazolecarboxamide;

4-Methyl-2-[[(2-naphthalenylamino)carbonyl]amino]-N-(2,4,6-trimethylphenyl)-5-thiazolecarboxamide;

2-[[(1H-Indol-5-ylamino)carbonyl]amino]-4-methyl-N-(2,4,6-trimethylphenyl)-5-thiazolecarboxamide;

2-[[(1,3-Benzodioxol-5-ylamino)carbonyl]amino]-4-methyl-N-(2,4,6-trimethylphenyl)-5-thiazolecarboxamide

4-Methyl-2-[[(2-pyrazinylamino)carbonyl]amino]-N-(2,4,6-trimethylphenyl)-5-thiazolecarboxamide;

2-[[[(5-Chloro-2-pyridinyl)amino]carbonyl]amino]-4-methyl-N-(2,4,6-trimethylphenyl)-5-thiazolecarboxamide;

4-Methyl-2-[[[(6-methyl-2-pyridinyl)amino]carbonyl]amino]-N-(2,4,6-trimethylphenyl)-5-thiazolecarboxamide;

4-Methyl-2-[[[(2-methyl-4-quinolinyl)amino]carbonyl]amino]-N-(2,4,6-trimethylphenyl)-5-thiazolecarboxamide;

2-[[[(2,3-Dihydro-1,4-benzodioxin-6-yl)amino]carbonyl]amino]-4-methyl-N-(2,4,6-trimethylphenyl)-5-thiazolecarboxamide;

2-[[([1,1′-Biphenyl]-2-ylamino)carbonyl]amino]-4-methyl-N-(2,4,6-trimethylphenyl)-5-thiazolecarboxamide;

2-[[[(4-Methoxy-2-methylphenyl)amino]carbonyl]amino]-4-methyl-N-(2,4,6-trimethylphenyl)-5-thiazolecarboxamide

4-Methyl-N-(2,4,6-trimethylphenyl)-2-[[[(2,4,6-trimethylphenyl)amino]carbonyl]amino]-5-thiazolecarboxamide;

2-[[[[2-(2-Hydroxyethyl)phenyl]amino]carbonyl]amino]-4-methyl-N-(2,4,6-trimethylphenyl)-5-thiazolecarboxamide;

2-[[[(3-Methoxyphenyl)amino]carbonyl]amino]-4-methyl-N-(2,4,6-trimethylphenyl)-5-thiazolecarboxamide;

2-[[[(4-Methoxy[1,1′-biphenyl]-3-yl)amino]carbonyl]amino]-4-methyl-N-(2,4,6-trimethylphenyl)-5-thiazolecarboxamide;

2-[[[(3-Acetylphenyl)amino]carbonyl]amino]-4-methyl-N-(2,4,6-trimethylphenyl)-5-thiazolecarboxamide;

2-[[[(4-Cyanophenyl)amino]carbonyl]amino]-4-methyl-N-(2,4,6-trimethylphenyl)-5-thiazolecarboxamide;

2-[[[[4-Fluoro-2-(trifluoromethyl)phenyl]amino]carbonyl]amino]-4-methyl-N-(2,4,6-trimethylphenyl)-5-thiazolecarboxamide

2-[[[(4-Hexyloxyphenyl)amino]carbonyl]amino]-4-methyl-N-(2,4,6-trimethylphenyl)-5-thiazolecarboxamide;

4-[[[[4-Methyl-5-[[(2,4,6-trimethylphenyl)amino]carbonyl]-2-thiazolyl]amino]carbonyl]amino]benzoic acid ethyl ester;

2-[[[(4-Decylphenyl)amino]carbonyl]amino]-4-methyl-N-(2,4,6-trimethylphenyl)-5-thiazolecarboxamide;

4-Methyl-2-[[[(4-propylphenyl)amino]carbonyl]amino]-N-(2,4,6-trimethylphenyl)-5-thiazolecarboxamide

4-Methyl-2-[[[(3,4,5-trimethoxyphenyl)amino]carbonyl]amino]-N-(2,4,6-trimethylphenyl)-5-thiazolecarboxamide;

4-Methyl-2-[[[[4-[[(5-methyl-3-isoxazolyl)amino]sulfonyl]phenyl]amino]carbonyl]amino]-N-(2,4,6-trimethylphenyl)-5-thiazolecarboxamide;

4-[[[[4-Methyl-5-[[(2,4,6-trimethylphenyl)amino]carbonyl]-2-thiazolyl]amino]carbonyl]amino]benzoic acid butyl ester;

2-[[(1-Isoquinolinylamino)carbonyl]amino]-4-methyl-N-(2,4,6-trimethylphenyl)-5-thiazolecarboxamide;

4-Methyl-2-[[[[2-[(phenylmethyl)thio]phenyl]amino]carbonyl]amino]-N-(2,4,6-trimethylphenyl)-5-thiazolecarboxamide;

4-Methyl-2-[[[[4-[(5-phenoxypentyl)oxy]phenyl]amino]carbonyl]amino]-N-(2,4,6-trimethylphenyl)-5-thiazolecarboxamide;

2-[[[[5-(1,1-Dimethylpropyl)-2-methoxyphenyl]amino]carbonyl]amino]-4-methyl-N-(2,4,6-trimethylphenyl)-5-thiazolecarboxamide;

2-[[[(1,2-Dihydro-5-acenaphthylenyl)amino]carbonyl]amino]-4-methyl-N-(2,4,6-trimethylphenyl)-5-thiazolecarboxamide;

4-Methyl-2-[[[(3-phenoxyphenyl)amino]carbonyl]amino]-N-(2,4,6-trimethylphenyl)-5-thiazolecarboxamide

4-Methyl-2-[[[[2-(4-morpholinyl)phenyl]amino]carbonyl]amino]-N-(2,4,6-trimethylphenyl)-5-thiazolecarboxamide;

4-Methyl-2-[[[[2-(1-piperidinyl)phenyl]amino]carbonyl]amino]-N-(2,4,6-trimethylphenyl)-5-thiazolecarboxamide;

2-[[[(1-Acetyl-2,3-dihydro-1H-indol-6-yl)amino]carbonyl]amino]-4-methyl-N-(2,4,6-trimethylphenyl)-5-thiazolecarboxamide;

2-[[[(2-Bromo-5-methoxyphenyl)amino]carbonyl]amino]-4-methyl-N-(2,4,6-trimethylphenyl)-5-thiazolecarboxamide;

2-[[[(2,3-Dimethyl-1H-indol-5-yl)amino]carbonyl]amino]-4-methyl-N-(2,4,6-trimethylphenyl)-5-thiazolecarboxamide;

4-Methyl-2-[[[[2-[[(1-methylethyl)amino]carbonyl]phenyl]amino]carbonyl]amino]-N-(2,4,6-trimethylphenyl)-5-thiazolecarboxamide;

2-[[[(3-Bromo-2-methylphenyl)amino]carbonyl]amino]-4-methyl-N-(2,4,6-trimethylphenyl)-5-thiazolecarboxamide;

2-[[[(4-Methoxybutyl)amino]carbonyl]amino]-4-methyl-N-(2,4,6-trimethylphenyl)-5-thiazolecarboxamide

2-[[[(3,3-Dimethylbutyl)amino]carbonyl]amino]-4-methyl-N-(2,4,6-trimethylphenyl)-5-thiazolecarboxamide;

4-Methyl-2-[[[(2-methylbutyl)amino]carbonyl]amino]-N-(2,4,6-trimethylphenyl)-5-thiazolecarboxamide;

4-Methyl-2-[[[(3-methylbutyl)amino]carbonyl]amino]-N-(2,4,6-trimethylphenyl)-5-thiazolecarboxamide;

2-[[[(2-Methoxyethyl)amino]carbonyl]amino]-4-methyl-N-(2,4,6-trimethylphenyl)-5-thiazolecarboxamide;

2-[[[[2-(Dimethylamino)ethyl]amino]carbonyl]amino]-4-methyl-N-(2,4,6-trimethylphenyl)-5-thiazolecarboxamide;

4-Methyl-2-[[[[2-(methylthio)ethyl]amino]carbonyl]aminno]-N-(2,4,6-trimethylphenyl)-5-thiazolecarboxamide;

2-[[(Butylamino)carbonyl]amino]-N-(2,3-dihydro-1H-inden-5-yl)-4-methyl-5-thiazolecarboxamide;

2-[[(Butylamino)carbonyl]amino]-N-2-naphthalenyl-4-methyl-5-thiazolecarboxamide;

2-[[(Butylamino)carbonyl]amino]-N-(3-hydroxy-2-naphthalenyl)-4-methyl-5-thiazolecarboxamide;

2-[[(Butylamino)carbonyl]amino]-N-(2-fluoro-5-methylphenyl)-4-methyl-5-thiazolecarboxamide;

2-[[(Butylamino)carbonyl]amino]-N-(2,6-dimethylphenyl)-4-methyl-5-thiazolecarboxamide;

N-(3-Bromo-2,4,6-trimethylphenyl)-2-[[(butylamino)carbonyl]amino]-4-methyl-5-thiazolecarboxamide;

2-[[(Butylamino)carbonyl]amino]-N-[2,6-dimethyl-3-(1-methylethyl)phenyl]-4-methyl-5-thiazolecarboxamide

N-(2-Bromo-4,6-dimethylphenyl)-2-[[(butylamino)carbonyl]amino]-4-methyl-5-thiazolecarboxamide;

3-[[[2-[[(Butylamino)carbonyl]amino]-4-methyl-5-thiazolyl]carbonyl]amino]-4-methyl-2-thiophenecarboxylic acid methyl ester;

2-[[(Butylamino)carbonyl]amino]-4-methyl-N-(2-methyl-6-quinolinyl)-5-thiazolecarboxamide;

2-[[(Butylamino)carbonyl]amino]-N-(2,6-dimethoxyphenyl)-4-methyl-5-thiazolecarboxamide;

2-[[(Butylamino)carbonyl]amino]-N-(4-methoxy-2-naphthalenyl)-4-methyl-5-thiazolecarboxamide;

2-[[(Butylamino)carbonyl]amino]-N-(2-methyl-1-naphthalenyl)-4-methyl-5-thiazolecarboxamide;

2-[[(Butylamino)carbonyl]amino]-N-[4-(dimethylamino)-2,3,5,6-tetramethylphenyl]-4-methyl-5-thiazolecarboxamide;

2-[[(Butylamino)carbonyl]amino]-N-(6-methyl-5-quinolinyl)-4-methyl-5-thiazolecarboxamide;

2-[[(Butylamino)carbonyl]amino]-N-[2-(2-hydroxyethyl)-6-methylphenyl]-4-methyl-5-thiazolecarboxamide;

2-[[(Butylamino)carbonyl]amino]-N-(2,6-dimethyl-3-nitrophenyl)-4-methyl-5-thiazolecarboxamide;

N-(2-Bromo-3,4,6-trimethylphenyl)-2-[[(butylamino)carbonyl]amino]-4-methyl-5-thiazolecarboxamide;

N-(2-Acetyl-6-hydroxyphenyl)-2-[[(butylamino)carbonyl]amino]-4-methyl-5-thiazolecarboxamide;

[4-[[[2-[[(Butylamino)carbonyl]amino]-4-methyl-5-thiazolyl]carbonyl]amino]-2,3,5,6-tetramethylphenyl]carbamic acid 1,1-dimethylethyl ester;

2-[[(Butylamino)carbonyl]amino]-N-(2,6-dichlorophenyl)-4-methyl-5-thiazolecarboxamide;

N-(4-Amino-2,3,5,6-tetramethylphenyl)-2-[[(butylamino)carbonyl]amino]-4-methyl-5-thiazolecarboxamide;

N-[5-(Acetylamino)-2,4-dimethylphenyl]-2-[[(butylamino)carbonyl]amino]-4-methyl-5-thiazolecarboxamide;

N-(4-Bromo-2,6-dimethylphenyl)-2-[[(butylamino)carbonyl]amino]-4-methyl-5-thiazolecarboxamide;

2-[[(Butylamino)carbonyl]amino]-N-(2-chloro-6-methylphenyl)-4-methyl-5-thiazolecarboxamide;

4-Methyl-2-[(methylsulfonyl)amino]-N-(2,4,6-trimethylphenyl)-5-thiazolecarboxamide;

4-Methyl-2-[[(phenylamino)thiocarbonyl]amino]-N-(2,4,6-trimethylphenyl)-5-thiazolecarboxamide;

2-[[(Ethylamino)carbonyl]amino]-4-methyl-N-(2,4,6-trimethylphenyl)-5-thiazolecarboxamide;

N-(2-Chloro-6-methylphenyl)-2-[(cyclopropylcarbonyl)amino]-5-thiazolecarboxamide;

2-[[[(1,1-Dimethylethyl)amino]carbonyl]amino]-N-(2-chloro-6-methylphenyl)-5-thiazolecarboxamide;

2-[[(1,1-Dimethylethoxy)carbonyl]amino]-4-methyl-N-(2,4,6-trimethylphenyl)-5-thiazoleacetamide;

2-Amino-4-methyl-N-(2,4,6-trimethylphenyl)-5-thiazoleacetamide;

N-(2-Chloro-6-methylphenyl)-2-[(4,6-dimethyl-2-pyridinyl)amino]-5-thiazolecarboxamide;

N-(2-Chloro-6-methylphenyl)-2-[(4-ethyl-2-pyridinyl)amino]-5-thiazolecarboxamide;

N-(2-Chloro-6-methylphenyl)-2-[(2,6-dimethyl-4-pyrimidinyl)amino]-5-thiazolecarboxamide;

N-(2-Chloro-6-methylphenyl)-2-(3-pyridazinylamino)-5-thiazolecarboxamide;

N-(2-Chloro-6-methylphenyl)-4-methyl-2-[(2-thienylcarbonyl)amino]-5-thiazolecarboxamide;

N-(2-Chloro-6-methylphenyl)-2-[(cyclopropylcarbonyl)amino]-4-methyl-5-thiazolecarboxamide;

N-(2-Chloro-6-methylphenyl)-4-methyl-2-[(2-furanylcarbonyl)amino]-5-thiazolecarboxamide;

N-(2-Chloro-6-methylphenyl)-4-methyl-2-[(3-thienylcarbonyl)amino]-5-thiazolecarboxamide;

N-(2-Chloro-6-methylphenyl)-4-methyl-2-[(3-furanylcarbonyl)amino]-5-thiazolecarboxamide;

trans-N-(2-Chloro-6-methylphenyl)-4-methyl-2-[[(2-phenylcyclopropyl)carbonyl]amino]-5-thiazolecarboxamide;

N-(2-Chloro-6-methylphenyl)-4-methyl-2-[[(2-methylcyclopropyl)carbonyl]amino]-5-thiazolecarboxamide;

N-(2-Chloro-6-methylphenyl)-2-[(cyclobutylcarbonyl)amino]-4-methyl-5-thiazolecarboxamide;

N-(2-Chloro-6-methylphenyl)-2-[(cyclopentylcarbonyl)amino]-4-methyl-5-thiazolecarboxamide;

2-(Benzoylamino)-N-(2-chloro-6-methylphenyl)-4-methyl-5-thiazolecarboxamide;

N-(2-Chloro-6-methylphenyl)-2-[(1-oxopropyl)amino]-5-thiazolecarboxamide;

N-(2-Chloro-6-methylphenyl)-2-[(1-oxobutyl)amino]-5-thiazolecarboxamide;

N-(2-Chloro-6-methylphenyl)-2-[(2-ethyl-1-oxobutyl)amino]-5-thiazolecarboxamide;

N-(2-Chloro-6-methylphenyl)-2-[[(1-phenylcyclopropyl)carbonyl]amino]-5-thiazolecarboxamide;

N-(2-Chloro-6-methylphenyl)-2-[[(1-methylcyclopropyl)carbonyl]amino]-5-thiazolecarboxamide;

N-(2-Chloro-6-methylphenyl)-2-[[(2,2-dichloro-1-methylcyclopropyl)carbonyl]amino]-5-thiazolecarboxamide;

N-(2-Chloro-6-methylphenyl)-2-[[(2-methylcyclopropyl)carbonyl]amino]-5-thiazolecarboxamide;

N-(2-Chloro-6-methylphenyl)-2-[[(1-hydroxycyclopropyl)carbonyl]amino]-5-thiazolecarboxamide;

N-(2-Chloro-6-methylphenyl)-2-[[(2,2,3,3-tetramethylcyclopropyl)carbonyl]amino]-5-thiazolecarboxamide;

N-(2-Chloro-6-methylphenyl)-2-[[(1-cyanocyclopropyl)carbonyl]amino]-5-thiazolecarboxamide;

N-(2-Chloro-6-methylphenyl)-2-[(cyclobutylcarbonyl)amino]-5-thiazolecarboxamide;

N-(2-Chloro-6-methylphenyl)-2-[(cyclopentylcarbonyl)amino]-5-thiazolecarboxamide;

N-(2-Chloro-6-methylphenyl)-2-[(cyclohexylcarbonyl)amino]-5-thiazolecarboxamide;

N-(2-Chloro-6-methylphenyl)-2-[(phenylacetyl)amino]-5-thiazolecarboxamide;

N-(2-Chloro-6-methylphenyl)-2-[(cyclohexylacetyl)amino]-5-thiazolecarboxamide;

N-(2-Chloro-6-methylphenyl)-2-[(4-pyridinylacetyl)amino]-5-thiazolecarboxamide;

N-(2-Chloro-6-methylphenyl)-2-[[(2,5-dimethyl-1H-pyrrol-3-yl)carbonyl]amino]-5-thiazolecarboxamide;

N-(2-Chloro-6-methylphenyl)-2-[(2-pyridinylcarbonyl)amino]-5-thiazolecarboxamide;

N-(2-Chloro-6-methylphenyl)-2-[(3-pyridinylcarbonyl)amino]-5-thiazolecarboxamide;

N-(2-Chloro-6-methylphenyl)-2-[(4-pyridinylcarbonyl)amino]-5-thiazolecarboxamide;

N-(2-Chloro-6-methylphenyl)-2-[(3-thienylcarbonyl)amino]-5-thiazolecarboxamide;

N-(2-Chloro-6-methylphenyl)-2-[(2-thienylcarbonyl)amino]-5-thiazolecarboxamide;

N-(2-Chloro-6-methylphenyl)-2-[(2-furanylcarbonyl)amino]-5-thiazolecarboxamide;

N-(2-Chloro-6-methylphenyl)-2-[(3-furanylcarbonyl)amino]-5-thiazolecarboxamide;

trans-N-(2-Chloro-6-methylphenyl)-2-[[(2-phenylcyclopropyl)carbonyl]amino]-5-thiazolecarboxamide;

N-(2-Chloro-6-methylphenyl)-2-[(2-methyl-1-oxopentyl)amino]-5-thiazolecarboxamide;

2-(Benzoylamino)-N-(2-chloro-6-methylphenyl)-5-thiazolecarboxamide;

2-[(Cyclopropylcarbonyl)amino]-N-(2,6-dimethylphenyl)-5-thiazolecarboxamide;

2-[(Cyclopropylcarbonyl)amino]-N-(2,4,6-trimethylphenyl)-5-thiazolecarboxamide;

N-(2-Chloro-4,6-dimethylphenyl)-2-[(cyclopropylcarbonyl)amino]-5-thiazolecarboxamide;

[4-[2-Oxo-2-[(2,4,6-trimethylphenyl)amino]ethyl]-2-thiazolyl]carbamic acid 1,1-dimethylethyl ester;

2-Amino-N-(2,4,6-trimethylphenyl)-4-thiazoleacetamide;

5-Amino-2-methyl-N-(2,4,6-trimethylphenyl)benzamide;

2-Amino-5-chloro-N-(2,4,6-trimethylphenyl)-4-pyrimidinecarboxamide;

[4-Methyl-5-[[(2,4,6-trimethylphenyl)amino]carbonyl]-2-oxazolyl]carbamic acid 1,1-dimethylethyl ester;

2-Amino-4-(methyl)-N-(2,4,6-trimethylphenyl)-5-oxazolecarboxamide, trifluoroacetate (1:1);

2-Amino-N-(2,4,6-trimethylphenyl)-5-pyridinecarboxamide;

3-Amino-N-(2,4,6-trimethylphenyl)-4-pyridinecarboxamide;

N-(2-Chloro-6-methylphenyl)-2-[(4-methyl-2-pyridinyl)amino]-5-thiazolecarboxamide;

2-[(6-Amino-2-pyridinyl)amino]-N-(2-chloro-6-methylphenyl)-5-thiazolecarboxamide;

N-(2-Chloro-6-methylphenyl)-2-[(6-propyl-2-pyridinyl)amino]-5-thiazolecarboxamide;

N-(2-Chloro-6-methylphenyl)-2-[(6-ethyl-4-pyrimidinyl)amino]-5-thiazolecarboxamide;

′N-(2-Chloro-6-methylphenyl)-2-(2-pyridinylamino)-5-thiazolecarboxamide;

′N-(2-Chloro-6-methylphenyl)-2-[(6-methyl-2-pyridinyl)amino]-5-thiazolecarboxamide;

′N-(2-Chloro-6-methylphenyl)-2-[(5-methyl-2-pyridinyl)amino]-5-thiazolecarboxamide;

′N-(2-Chloro-6-methylphenyl)-2-[(4-methyl-2-pyridinyl)amino]-5-thiazolecarboxamide;

′N-(2-Chloro-6-methylphenyl)-2-[(3-methyl-2-pyridinyl)amino]-5-thiazolecarboxamide;

′2-[(5-Bromo-3-methyl-2-pyridinyl)amino]-N-(2-chloro-6-methylphenyl)-5-thiazolecarboxamide;

′2-[(6-Amino-2-pyridinyl)amino]-N-(2-chloro-6-methylphenyl)-5-thiazolecarboxamide;

′2-[(5-Bromo-2-pyridinyl)amino]-N-(2-chloro-6-methylphenyl)-5-thiazolecarboxamide;

′N-(2-Chloro-6-methylphenyl)-2-[[3-(phenylmethoxy)-2-pyridinyl]amino]-5-thiazolecarboxamide;

′N-(2-Chloro-6-methylphenyl)-2-[(5-chloro-2-pyridinyl)amino]-5-thiazolecarboxamide;

′N-(2-Chloro-6-methylphenyl)-2-[(6-ethyl-2-pyridinyl)amino]-5-thiazolecarboxamide;

′N-(2-Chloro-6-methylphenyl)-2-[(6-propyl-2-pyridinyl)amino]-5-thiazolecarboxamide;

′2-[(3-Bromo-5-methyl-2-pyridinyl)amino]-N-(2-chloro-6-methylphenyl)-5-thiazolecarboxamide;

′2-[(2-Amino-3-pyridinyl)amino]-N-(2-chloro-6-methylphenyl)-5-thiazolecarboxamide;

′2-[(3-Amino-2-pyridinyl)amino]-N-(2-chloro-6-methylphenyl)-5-thiazolecarboxamide;

′N-(2-Chloro-6-methylphenyl)-2-(4-pyridinylamino)-5-thiazolecarboxamide;

′N-(2-Chloro-6-methylphenyl)-2-(3-pyridinylamino)-5-thiazolecarboxamide;

′N-(2-Chloro-6-methylphenyl)-2-[(6-chloro-3-pyridinyl)amino]-5-thiazolecarboxamide;

′N-(2-Chloro-6-methylphenyl)-2-[(2-chloro-3-pyridinyl)amino]-5-thiazolecarboxamide;

′N-(2-Chloro-6-methylphenyl)-2-[(6-methoxy-3-pyridinyl)amino]-5-thiazolecarboxamide;

′N-(2-Chloro-6-methylphenyl)-2-[(3,5-dimethyl-2-pyrazinyl)amino]-5-thiazolecarboxamide;

′N-(2-Chloro-6-methylphenyl)-2-(phenylamino)-5-thiazolecarboxamide;

′N-(2-Chloro-6-methylphenyl)-2-[(3-ethylphenyl)amino]-5-thiazolecarboxamide;

′N-(2-Chloro-6-methylphenyl)-2-[(3,5-dimethylphenyl)amino]-5-thiazolecarboxamide;

′N-(2-Chloro-6-methylphenyl)-2-[(4,6-di methyl-2-pyrimidinyl)amino]-5-thiazolecarboxamide;

′N-(2-Chloro-6-methylphenyl)-2-[(6-ethyl-4-pyrimidinyl)amino]-5-thiazolecarboxamide;

′N-(2-Chloro-6-methylphenyl)-2-[(6-chloro-2-pyrazinyl)amino]-5-thiazolecarboxamide;

′2-[(3-Aminophenyl)amino]-N-(2-chloro-6-methylphenyl)-5-thiazolecarboxamide;

′N-(2-Chloro-6-methylphenyl)-2-[(3-hydroxyphenyl)amino]-5-thiazolecarboxamide;

′2-[(3-Bromophenyl)amino]-N-(2-chloro-6-methylphenyl)-5-thiazolecarboxamide;

′N-(2,6-Dimethylphenyl)-2-(phenylamino)-5-thiazolecarboxamide;

′N-(2,6-Dimethylphenyl)-2-(methylphenylamino)-5-thiazolecarboxamide;

′N-(2,6-Dimethylphenyl)-2-(2-pyridinylamino)-5-thiazolecarboxamide;

′N-(2,6-Dimethylphenyl)-2-[(6-methyl-2-pyridinyl)amino]-5-thiazolecarboxamide;

′N-(2,6-Dimethylphenyl)-2-[(4-methyl-2-pyridinyl)amino]-5-thiazolecarboxamide;

′N-(2,6-Dimethylphenyl)-2-[(4-ethyl-2-pyridinyl)amino]-5-thiazolecarboxamide;

′N-(2,6-Dimethylphenyl)-2-[(4,6-dimethyl-2-pyridinyl)amino]-5-thiazolecarboxamide;

′2-[(6-Amino-2-pyridinyl)amino]-N-(2,6-dimethylphenyl)-5-thiazolecarboxamide;

′N-(2,6-Dimethylphenyl)-2-[(6-ethyl-2-pyridinyl)amino]-5-thiazolecarboxamide;

′N-(2,6-Dimethylphenyl)-2-[(6-propyl-2-pyridinyl)amino]-5-thiazolecarboxamide;

′2-[(2-Amino-3-pyridinyl)amino]-N-(2,6-dimethylphenyl)-5-thiazolecarboxamide;

′2-[(3-Amino-2-pyridinyl)amino]-N-(2,6-dimethylphenyl)-5-thiazolecarboxamide;

′2-[(6-Amino-2-methyl-4-pyrimidinyl)amino]-N-(2,6-dimethylphenyl)-5-thiazolecarboxamide;

′N-(2,6-Dimethylphenyl)-2-[[6-(4-morpholinyl)-3-pyridazinyl]amino]-5-thiazolecarboxamide;

′2-[(6-Chloro-3-pyridazinyl)amino]-N-(2,6-dimethylphenyl)-5-thiazolecarboxamide;

′N-(2,6-Dimethylphenyl)-2-(3-pyridazinylamino)-5-thiazolecarboxamide;

′2-[(3-Aminophenyl)amino]-N-(2,6-dimethylphenyl)-5-thiazolecarboxamide;

′2-[(3-Bromophenyl)amino]-N-(2,6-dimethylphenyl)-5-thiazolecarboxamide;

′2-(2-Pyridinylamino)-N-(2,4,6-trimethylphenyl)-5-thiazolecarboxamide;

′2-[(6-Methyl-2-pyridinyl)amino]-N-(2,4,6-trimethylphenyl)-5-thiazolecarboxamide;

′2-[(5-Methyl-2-pyridinyl)amino]-N-(2,4,6-trimethylphenyl)-5-thiazolecarboxamide;

′2-[(4-Methyl-2-pyridinyl)amino]-N-(2,4,6-trimethylphenyl)-5-thiazolecarboxamide;

′2-[(3-Methyl-2-pyridinyl)amino]-N-(2,4,6-trimethylphenyl)-5-thiazolecarboxamide;

′2-[(5-Bromo-2-pyridinyl)amino]-N-(2,4,6-trimethylphenyl)-5-thiazolecarboxamide;

′2-[(5-Chloro-2-pyridinyl)amino]-N-(2,4,6-trimethylphenyl)-5-thiazolecarboxamide;

′2-[(6-Methoxy-3-pyridinyl)amino]-N-(2,4,6-trimethylphenyl)-5-thiazolecarboxamide;

′2-[(4-Ethyl-2-pyridinyl)amino]-N-(2,4,6-trimethylphenyl)-5-thiazolecarboxamide;

′2-[(6-Ethyl-2-pyridinyl)amino]-N-(2,4,6-trimethylphenyl)-5-thiazolecarboxamide;

′2-[(6-Chloro-3-pyridinyl)amino]-N-(2,4,6-trimethylphenyl)-5-thiazolecarboxamide;

′2-[(2,6-Dimethyl-4-pyrimidinyl)amino]-N-(2,4,6-trimethylphenyl)-5-thiazolecarboxamide;

′2-[(4-Methyl-2-pyrimidinyl)amino]-N-(2,4,6-trimethylphenyl)-5-thiazolecarboxamide;

′2-(2-Pyrazinylamino)-N-(2,4,6-trimethylphenyl)-5-thiazolecarboxamide

′2-[(6-Chloro-2-pyrazinyl)amino]-N-(2,4,6-trimethylphenyl)-5-thiazolecarboxamide;

′2-[(3,5-Dimethyl-2-pyrazinyl)amino]-N-(2,4,6-trimethylphenyl)-5-thiazolecarboxamide;

′N-(2-Chloro-6-methylphenyl)-2-[[2-methyl-6-[[2-(4-morpholinyl)ethyl]amino]-4-pyrimidinyl]amino]-5-thiazolecarboxamide;

′N-(2-Chloro-6-methylphenyl)-2-[[2-methyl-6-[[3-(4-morpholinyl)propyl]amino]-4-pyrimidinyl]amino]-5-thiazolecarboxamide;

′N-(2-Chloro-6-methylphenyl)-2-[[2-methyl-6-[methyl[3-(methylamino)propyl]amino]-4-pyrimidinyl]amino]-5-thiazolecarboxamide;

′N-(2-Chloro-6-methylphenyl)-2-[[2-methyl-6-[[2-(tetrahydro-2-oxo-1H-imidazol-1-yl)ethyl]amino]-4-pyrimidinyl]amino]-5-thiazolecarboxamide;

′N-(2-Chloro-6-methylphenyl)-2-[[2-methyl-6-[(2-1H-imidazol-4-ylethyl)amino]-4-pyrimidinyl]amino]-5-thiazolecarboxamide;

′N-(2-Chloro-6-methylphenyl)-2-[[2-methyl-6-(4-morpholinyl)-4-pyrimidinyl]amino]-5-thiazolecarboxamide;

′N-(2-Chloro-6-methylphenyl)-2-[[6-[[[(2R)-1-ethyl-2-pyrrolidinyl]methyl]amino]-2-methyl-4-pyrimidinyl]amino]-5-thiazolecarboxamide;

′N-(2-Chloro-6-methylphenyl)-2-[[6-[[[(2S)-1-ethyl-2-pyrrolidinyl]methyl]amino]-2-methyl-4-pyrimidinyl]amino]-5-thiazolecarboxamide;

′2-[[6-[(2S)-2-(Aminocarbonyl)-1-pyrrolidinyl]-2-methyl-4-pyrimidinyl]amino]-N-(2-chloro-6-methylphenyl)-5-thiazolecarboxamide;

′N-(2-Chloro-6-methylphenyl)-2-[[6-[(2-hydroxyethyl)amino]-2-methyl-4-pyrimidinyl]amino]-5-thiazolecarboxamide;

′N-(2-Chloro-6-methylphenyl)-2-[[6-[4-(hydroxymethyl)-1-piperidinyl]-2-methyl-4-pyrimidinyl]amino]-5-thiazolecarboxamide;

′N-(2-Chloro-6-methylphenyl)-2-[[6-[4-(2-hydroxyethyl)-1-piperazinyl]-2-methyl-4-pyrimidinyl]amino]-5-thiazolecarboxamide;

′1-[6-[[5-[[(2-Chloro-6-methylphenyl)amino]carbonyl]-2-thiazolyl]amino]-2-methyl-4-pyrimidinyl]-4-piperidinecarboxamide;

′N-(2-Chloro-6-methylphenyl)-2-[[2-methyl-6-[(3S)-3-methyl-1-piperazinyl]-4-pyrimidinyl]amino]-5-thiazolecarboxamide;

′2-[[6-[3-(Acetylamino)-1-pyrrolidinyl]-2-methyl-4-pyrimidinyl]amino]-N-(2-chloro-6-methylphenyl)-5-thiazolecarboxamide;

′N-(2-Chloro-6-methylphenyl)-2-[[6-[[2-(1-methyl-2-pyrrolidinyl)ethyl]amino]-2-methyl-4-pyrimidinyl]amino]-5-thiazolecarboxamide;

′N-(2-Chloro-6-methylphenyl)-2-[[2-methyl-6-[[(5-methyl-2-pyrazinyl)methyl]amino]-4-pyrimidinyl]amino]-5-thiazolecarboxamide;

′N-(2-Chloro-6-methylphenyl)-2-[[2-methyl-6-[[2-(1H-1,2,3-triazol-1-yl)ethyl]amino]-4-pyrimidinyl]amino]-5-thiazolecarboxamide;

′N-(2-Chloro-6-methylphenyl)-2-[[6-[[2-(4-morpholinyl)ethyl]amino]-4-pyrimidinyl]amino]-5-thiazolecarboxamide;

′N-(2-Chloro-6-methylphenyl)-2-[[6-[[2-(dimethylamino)ethyl]amino]-4-pyrimidinyl]amino]-5-thiazolecarboxamide;

′N-(2-Chloro-6-methylphenyl)-2-[[6-[[2-(tetrahydro-2-oxo-1H-imidazol-1-yl)ethyl]amino]-4-pyrimidinyl]amino]-5-thiazolecarboxamide;

′N-(2-Chloro-6-methylphenyl)-2-[[6-[methyl [2-(methylamino)ethyl]amino]-4-pyrimidinyl]amino]-5-thiazolecarboxamide;

′N-(2-Chloro-6-methylphenyl)-2-[[6-[[2-(1-methyl-2-pyrrolidinyl)ethyl]amino]-4-pyrimidinyl]amino]-5-thiazolecarboxamide;

′N-(2-Chloro-6-methylphenyl)-2-[[6-[[2-(1-pyrrolidinyl)ethyl]amino]-4-pyrimidinyl]amino]-5-thiazolecarboxamide;

′N-(2-Chloro-6-methylphenyl)-2-[[6-[[(1-ethyl-2-pyrrolidinyl)methyl]amino]-4-pyrimidinyl]amino]-5-thiazolecarboxamide;

′N-(2-Chloro-6-methylphenyl)-2-[[6-[(4-piperidinylmethyl)amino]-4-pyrimidinyl]amino]-5-thiazolecarboxamide;

′2-[[6-[[2-(Acetylamino)ethyl]amino]-4-pyrimidinyl]amino]-N-(2-chloro-6-methylphenyl)-5-thiazolecarboxamide;

′N-(2-Chloro-6-methylphenyl)-2-[[6-[[2-(1H-1,2,3-triazol-1-yl)ethyl]amino]-4-pyrimidinyl]amino]-5-thiazolecarboxamide;

′N-(2-Chloro-6-methylphenyl)-2-[[6-(4-morpholinyl)-4-pyrimidinyl]amino]-5-thiazolecarboxamide;

′N-(2-Chloro-6-methylphenyl)-2-[[6-[[2-(4-morpholinyl)ethyl]amino]-2-pyridinyl]amino]-5-thiazolecarboxamide;

′N-(2-Chloro-6-methylphenyl)-2-[[6-[[3-(4-morpholinyl)propyl]amino]-2-pyridinyl]amino]-5-thiazolecarboxamide;

′N-(2-Chloro-6-methylphenyl)-2-[[6-[methyl [3-(methylamino)propyl]amino]-2-pyridinyl]amino]-5-thiazolecarboxamide;

′N-(2-Chloro-6-methylphenyl)-2-[[6-[(3S)-3-methyl-1-piperazinyl]-2-pyridinyl]amino]-5-thiazolecarboxamide;

′N-(2-Chloro-6-methylphenyl)-2-[[6-[(3-1H-imidazol-1-ylpropyl)amino]-2-pyridinyl]amino]-5-thiazolecarboxamide;

′N-(2-Chloro-6-methylphenyl)-2-[[6-[(2-hydroxyethyl)amino]-2-pyridinyl]amino]-5-thiazolecarboxamide;

′N-(2-Chloro-6-methylphenyl)-2-[[6-[(2-1H-imidazol-1-ylethyl)amino]-2-pyridinyl]amino]-5-thiazolecarboxamide;

′N-(2-Chloro-6-methylphenyl)-2-[[6-(4-morpholinyl)-2-pyridinyl]amino]-5-thiazolecarboxamide;

′N-(2-Chloro-6-methylphenyl)-2-[[6-[[2-(4-morpholinyl)ethyl]amino]-2-pyrazinyl]amino]-5-thiazolecarboxamide;

′N-(2-Chloro-6-methylphenyl)-2-[[6-[[3-(4-morpholinyl)propyl]amino]-2-pyrazinyl]amino]-5-thiazolecarboxamide;

′N-(2-Chloro-6-methylphenyl)-2-[[6-(4-morpholinyl)-2-pyrazinyl]amino]-5-thiazolecarboxamide;

′N-(2-Chloro-6-methylphenyl)-2-[[6-[(3S)-3-methyl-1-piperazinyl]-2-pyrazinyl]amino]-5-thiazolecarboxamide;

′N-(2-Chloro-6-methylphenyl)-2-[[6-(3-hydroxy-1-pyrrolidinyl)-2-pyrazinyl]amino]-5-thiazolecarboxamide;

′N-(2-Chloro-6-methylphenyl)-2-[[6-(1H-imidazol-1-yl)-2-pyrazinyl]amino]-5-thiazolecarboxamide;

′N-(2-Chloro-6-methylphenyl)-2-[[6-(3-hydroxy-1-pyrrolidinyl)-3-pryidazinyl]amino]-5-thiazolecarboxamide;

′N-(2-Chloro-6-methylphenyl)-2-[[6-(1H-imidazol-1-yl)-3-pyridazinyl]amino]-5-thiazolecarboxamide;

′N-(2-Chloro-6-methylphenyl)-2-[[3-(methylamino)-2-pyrazinyl]amino]-5-thiazolecarboxamide;

′N-(2-Chloro-6-methylphenyl)-2-[[3-(3-hydroxy-1-pyrrolidinyl)-2-pyrazinyl]amino]-5-thiazolecarboxamide;

′N-(2-Chloro-6-methylphenyl)-2-[[3-(cyclopropylamino)-2-pyrazinyl]amino]-5-thiazolecarboxamide;

′N-(2-Chloro-6-methylphenyl)-2-[[3-(4-morpholinyl)-2-pyrazinyl]amino]-5-thiazolecarboxamide;

′N-(2-Chloro-6-methylphenyl)-2-[[3-[[2-(4-morpholinyl)ethyl]amino]-2-pyrazinyl]amino]-5-thiazolecarboxamide;

′2-[[3-[[2-(Acetylamino)ethyl]amino]-2-pyrazinyl]amino]-N-(2-chloro-6-methylphenyl)-5-thiazolecarboxamide;

′N-(2-Chloro-6-methylphenyl)-2-(cyclohexylamino)-5-thiazolecarboxamide;

′N-(2-Chloro-6-methylphenyl)-2-(methylamino)-5-thiazolecarboxamide;

′N-(2-Chloro-6-methylphenyl)-2-(cyclopropylamino)-5-thiazolecarboxamide;

′N-(2-Chloro-6-methylphenyl)-2-[(phenylmethyl)amino]-5-thiazolecarboxamide;

′2-[[2-(Acetylamino)ethyl]amino]-N-(2-chloro-6-methylphenyl)-5-thiazolecarboxamide;

′N-(2-Chloro-6-methylphenyl)-2-[[(1R)-1-(hydroxymethyl)-3-methylbutyl]amino]-5-thiazolecarboxamide;

′N-(2-Chloro-6-methylphenyl)-2-[[6-(methoxymethyl)-4-pyrimidinyl]amino]-5-thiazolecarboxamide;

′N-(2-Chloro-6-methylphenyl)-2-[[6-hydroxymethyl)-4-pyrimidinyl]amino]-5-thiazolecarboxamide;

′N-(2-Chloro-6-methylphenyl)-2-[[6-(4-morpholinylmethyl)-4-pyrimidinyl]amino]-5-thiazolecarboxamide;

′N-(2-Chloro-6-methylphenyl)-2-[[6-[[[2-(dimethylamino)-ethyl]amino]methyl]-4-pyrimidinyl]amino]-5-thiazolecarboxamide;

′N-(2-Chloro-6-methylphenyl)-2-[[6-[[[2-(4-morpholinyl)ethyl]amino]methyl]-4-pyrimidinyl]amino]-5-thiazolecarboxamide;

′N-(2-Chloro-6-methylphenyl)-2-[[6-[[[3-(4-morpholinyl)propyl]-amino]methyl]-4-pyrimidinyl]amino]-5-thiazolecarboxamide;

′N-(2-Chloro-6-methylphenyl)-2-[[6-[[[3-(2-oxo-1-pyrrolidinyl)propyl]amino]methyl]-4-pyrimidinyl]amino]-5-thiazolecarboxamide;

′N-(2-Chloro-6-methylphenyl)-2-[[6-[[(2-1H-imidazol-4-ylethyl)amino]methyl]-4-pyrimidinyl]amino]-5-thiazolecarboxamide;

′N-(2-Chloro-6-methylphenyl)-2-[[6-[[(3-1H-imidazol-1-ylpropyl)amino]methyl]-4-pyrimidinyl]amino]-5-thiazolecarboxamide;

′N-(2-Chloro-6-methylphenyl)-2-[[6-[[[2-(2-pyridinyl)ethyl]amino]methyl]-4-pyrimidinyl]amino]-5-thiazolecarboxamide;

′N-(2-Chloro-6-methylphenyl)-2-[[6-[[[2-(3-pyridinyl)ethyl]amino]methyl]-4-pyrimidinyl]amino]-5-thiazolecarboxamide;

′1-[[6-[[5-[[(2-Chloro-6-methylphenyl)amino]carbonyl]-2-thiazolyl]amino]-4-pyrimidinyl]methyl]-4-piperidinecarboxamide;

′2-[[6-[[[2-(Acetylamino)ethyl]amino]methyl]-4-pyrimidinyl]amino]-N-(2-chloro-6-methylphenyl)-5-thiazolecarboxamide;

′N-(2-Chloro-6-methylphenyl)-2-(2-naphthalenylamino)-5-thiazolecarboxamide;

′N-(2-Chloro-6-methylphenyl)-2-(2-quinolinylamino)-5-thiazolecarboxamide;

′N-(2-Chloro-6-methylphenyl)-2-(3-isoquinolinylamino)-5-thiazolecarboxamide;

′N-(2-Chloro-6-methylphenyl)-2-(2-quinoxalinylamino)-5-thiazolecarboxamide;

′N-(2-Chloro-6-methylphenyl)-4-methyl-2-[[2-methyl-6-(4-morpholinyl)-4-pyrimidinyl]amino]-5-thiazolecarboxamide;

′N-(2-Chloro-6-methylphenyl)-4-methyl-2-[[2-methyl-6-[[2-(4-morpholinyl)ethyl]amino]-4-pyrimidinyl]amino]-5-thiazolecarboxamide;

′2-[(2,6-Dimethyl-4-pyrimidinyl)amino]-N-phenyl-5-thiazolecarboxamide;

′2-[(2,6-Dimethyl-4-pyrimidinyl)methylamino]-N-(2-methylphenyl)-5-thiazolecarboxamide;

′2-[(2,6-Dimethyl-4-pyrimidinyl)amino]-N-(2-methylphenyl)-5-thiazolecarboxamide;

′N-(3,5-Dimethoxyphenyl)-2-[(2,6-dimethyl-4-pyrimidinyl)amino]-5-thiazolecarboxamide;

′N-[2,6-Bis(1-methylethyl)phenyl]-2-[(2,6-dimethyl-4-pyrimidinyl)amino]-5-thiazolecarboxamide;

′N-(2-Chloro-6-methylphenyl)-2-[2,6-dimethyl-4-pyrimidinyl)methylamino]-5-thiazolecarboxamide;

′N-(2-Chloro-6-methylphenyl)-2-[(2,6-dimethyl-4-pyrimidinyl)amino]-N-methyl-5-thiazolecarboxamide;

N-,N-(2-Chloro-6-methylphenyl)-(4-methoxybenzyl)-2-[(6-bromo-2-pyridinyl)amino]-5-thiazolecarboxamide;

N-(2-Chloro-6-methylphenyl)-2-[(6-bromo-2-pyridinyl)amino-5-thiazolecarboxamide;

′N-(2-Chloro-6-methylphenyl)-2-[[6-[4-(2-furanylcarbonyl)-1-piperazinyl]-2-pyridinyl]amino]-5-thiazolecarboxamide;

′2-[[6-[[3-(1H-Benzimidazol-1-yl)propyl]amino]-2-pyridinyl]amino]-N-(2-chloro-6-methylphenyl)-5-thiazolecarboxamide;

′N-(2-Chloro-6-methylphenyl)-2-[[6-[[4-(1H-imidazol-1-yl)butyl]amino]-2-pyridinyl]amino]-5-thiazolecarboxamide;

′N-(2-Chloro-6-methylphenyl)-2-[[6-[[5-(1H-imidazol-1-yl)pentyl]amino]-2-pyridinyl]amino]-5-thiazolecarboxamide;

′N-(2-Chloro-6-methylphenyl)-2-[[6-[[3-(4-methyl-1-piperazinyl)propyl]amino]-2-pyridinyl]amino]-5-thiazolecarboxamide;

′N-(2-Chloro-6-methylphenyl)-2-[[6-[[4-(1H-imidazol-1-yl)phenyl]amino]-2-pyridinyl]amino]-5-thiazolecarboxamide;

′N-(2-Chloro-6-methylphenyl)-2-[[6-[[6-(1H-imidazol-1-yl)hexyl]amino]-2-pyridinyl]amino]-5-thiazolecarboxamide;

′N-(2-Chloro-6-methylphenyl)-2-[[6-[(3-1H-imidazol-1-ylpropyl)amino]-2-pyridinyl]amino]-5-thiazolecarboxamide;

′2-[[6-[[3-(1H-Imidazol-1-yl)propyl]amino]-2-pyridinyl]amino]-N-(4-methoxyphenyl)-5-thiazolecarboxamide;

′2-[[6-[[3-(1H-Imidazol-1-yl)propyl]amino]-2-pyridinyl]amino]-N-(4-phenoxyphenyl)-5-thiazolecarboxamide;

′N-(4-Chlorophenyl)-2-[[6-[[3-(1H-imidazol-1-yl)propyl]amino]-2-pyridinyl]amino]-5-thiazolecarboxamide;

′2-[[6-[[3-(1H-Imidazol-1-yl)propyl]amino]-2-pyridinyl]amino]-N-[1-(phenylmethyl)-1H-indazol-5-yl]-5-thiazolecarboxamide;

′N-(2-Ethylphenyl)-2-[[6-[[3-(1H-imidazol-1-yl)propyl]amino]-2-pyridinyl]amino]-5-thiazolecarboxamide;

′N-(2,6-Dimethoxyphenyl)-2-[[6-[[3-(1H-imidazol-1-yl)propyl]amino]-2-pyridinyl]amino]-5-thiazolecarboxamide;

′N-(2,4-Dimethoxyphenyl)-2-[[6-[[3-(1H-imidazol-1-yl)propyl]amino]-2-pyridinyl]amino]-5-thiazolecarboxamide;

′2-[[6-[[3-(1H-Imidazol-1-yl)propyl]amino]-2-pyridinyl]amino]-N-phenyl-5-thiazolecarboxamide;

′2-[[6-[[3-(1H-Imidazol-1-yl)propyl]amino]-2-pyridinyl]amino]-N-(2-methylphenyl)-5-thiazolecarboxamide;

′N-(2-Chlorophenyl)-2-[[6-[[3-(1H-imidazol-1-yl)propyl]amino]-2-pyridinyl]amino]-5-thiazolecarboxamide;

′N-(2,6-Diethylphenyl)-2-[[6-[[3-(1H-imidazol-1-yl)propyl]amino]-2-pyridinyl]amino]-5-thiazolecarboxamide;

′N-(2-Chloro-6-methylphenyl)-2-[[6-[[3-(1H-imidazol-1-yl)propyl]amino]-2-pyridinyl]amino]-4-methyl-5-thiazolecarboxamide;

′2-[[6-[[3-(1H-Imidazol-1-yl)propyl]amino]-2-pyridinyl]amino]-4-methyl-N-[1-(phenylmethyl)-1H-indazol-5-yl]-5-thiazolecarboxamide;

′N-(2-Chloro-6-methylphenyl)-2-[[3-[[3-(1H-imidazol-1-yl)propyl]amino]phenyl]amino]-5-thiazolecarboxamide;

′N-(2-Chloro-6-methylphenyl)-2-[[5-[[3-(1H-imidazol-1-yl)propyl]amino]-2-nitrophenyl]amino]-5-thiazolecarboxamide;

N-(2-Chloro-6-methylphenyl)-2-[(3,4,5-trimethoxy-phenyl)amino]-5-thiazolecarboxamide;

N-(2-Chloro-6-methyl-phenyl)-2-[(4-methoxy-phenyl)amino]-5-thiazolecarboxamide;

N-(2-Chloro-6-methyl-phenyl)-2-[(3-methoxy-phenyl)amino]-5-thiazolecarboxamide;

N-(2-Chloro-6-methyl-phenyl)-2-[(2-methoxy-phenyl)amino]-5-thiazolecarboxamide;

N-(2-Chloro-6-methyl-phenyl)-2-[(3,5-dimethoxyphenyl)amino]-5-thiazolecarboxamide;

N-(2-Chloro-6-methyl-phenyl)-2-[[4-(dimethylamino)-phenyl]amino]-5-thiazolecarboxamide;

N-(2-Chloro-6-methylphenyl)-2-[[4-(4-morpholinyl)phenyl]amino]-5-thiazolecarboxamide;

N-(2-Chloro-6-methylphenyl)-2-[[3-(carboxymethyl)-phenyl]amino]-5-thiazolecarboxamide;

N-(2-Chloro-6-methylphenyl)-2-[[3-(3-carboxypropyl)-phenyl]amino]-5-thiazolecarboxamide;

N-(2-Chloro-6-methylphenyl)-2-[[4-(carboxymethyl)phenyl]amino]-5-thiazolecarboxamide;

N-(2-Chloro-6-methylphenyl)-2-[(2-methyl-1H-benzimidazol-5-yl)amino]-5-thiazolecarboxamide;

N-(2-Chloro-6-methylphenyl)-2-[[1-[3-(1H-imidazol-1-yl)propyl]-1H-benzimidazol-4-yl]amino]-5-thiazolecarboxamide;

N-(2-Chloro-6-methylphenyl)-2-[[1-[2-(1H-imidazol-1-yl)ethyl]-1H-indazol-6-yl]amino]-5-thiazolecarboxamide;

N-(2-Chloro-6-methylphenyl)-2-[[2-[2-(1H-imidazol-1-yl)ethyl]-2H-indazol-6-yl]amino]-5-thiazolecarboxamide;

N-(2-Chloro-6-methylphenyl)-2-[(1-methyl-1H-benzimidazol-6-yl)amino]-5-thiazolecarboxamide;

N-(2-Chloro-6-methylphenyl)-2-[(1-methyl-1H-benzimidazol-5-yl)amino]-5-thiazolecarboxamide;

N-(2-Chloro-6-methylphenyl)-2-[[2-[3-(1H-imidazol-1-yl)propyl]amino]-1H-benzimidazol-5-yl]amino]-5-thiazolecarboxamide;

N-(2-Chloro-6-methylphenyl)-2-[[2-(4-morpholinylmethyl)-1H-benzimidazol-5-yl]amino]-5-thiazolecarboxamide;

N-(2-Chloro-6-methylphenyl)-2-[[2-(1H-imidazol-1-ylmethyl)-1H-benzimidazol-5-yl]amino]-5-thiazolecarboxamide;

N-(2-Chloro-6-methylphenyl)-2-[[3-[[5-(1H-imidazol-1-yl)-2-pyridinyl]amino]phenyl]amino]-5-thiazolecarboxamide;

N-(2-Chloro-6-methylphenyl)-2-[[3-[3-(1H-imidazol-1-yl)propoxy]phenyl]amino]-5-thiazolecarboxamide;

N-(2-Chloro-6-methylphenyl)-2-[[4-[3-(1H-imidazol-1-yl)propoxy]phenyl]amino]-5-thiazolecarboxamide;

N-(2-Chloro-6-methylphenyl)-2-[[3-[[[3-(1H-imidazol-1-yl)propyl]amino]sulfonyl]phenyl]amino]-5-thiazolecarboxamide; and

N-(2-Chloro-6-methylphenyl)-2-[[4-[[[3-(1H-imidazol-1-yl)propyl]amino]sulfonyl]phenyl]amino]-5-thiazolecarboxamide.

In one embodiment of the present invention, the anticancer agent is a MEK inhibitor having the following formula IV:

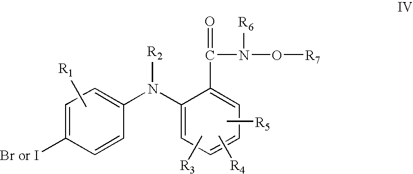

wherein

R₁ is hydrogen, hydroxy, C₁-C₈ alkyl, C₁-C₈ alkoxy, halo, trifluoromethyl, or CN;

R₂ is hydrogen;

R₃, R₄, and R₅ independently are hydrogen, hydroxy, halo, trifluoromethyl, C₁-C₈ alkyl, C₁-C₈ alkoxy, nitro, CN, or (O or NH)ₘ—(CH₂)ₙ—R₉, where R₉ is hydrogen, hydroxy, CO₂H or NR₁₀R₁₁;

n is 0 to 4;

m is 0 or 1;

R₁₀ and R₁₁ independently are hydrogen or C₁-C₈ alkyl, or taken together with the nitrogen to which they are attached can complete a 3- to 10-member cyclic ring optionally containing one, two, or three additional heteroatoms selected from O, S, NH, or N—C₁-C₈ alkyl;

R₆ is hydrogen, C₁-C₈ alkyl,

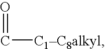

alkyl, aryl, aralkyl, or C₃-C₁₀ cycloalkyl;

R₇ is hydrogen, C₁-C₈ alkyl, C₂-C₈ alkenyl, C₂-C₈ alkynyl, C₃-C₁₀ (cycloalkyl or cycloalkyl optionally containing a heteroatom selected from O, S, or NR₉); and

wherein any of the foregoing alkyl, alkenyl, and alkynyl groups can be unsubstituted or substituted by cycloalkyl (or cycloalkyl optionally containing a heteroatom selected from O, S, or NR₉), aryl, aryloxy, heteroaryl, or heteroaryloxy; or R₆ and R₇ taken together with the N-0 to which they are attached can complete a 5- to 10-membered cyclic ring, optionally containing one, two, or three additional heteroatoms selected from O, S, or NR₁₀R₁₁.

Preferred MEK inhibitors are selected from the group consisting of:

4-Fluoro-N-hydroxy-2-(4-iodo-2-methyl-phenylamino)-benzamide;

4-Fluoro-2-(4-iodo-2-methyl-phenylamino)-N-(methoxy)-benzamide;

4-Fluoro-2-(4-iodo-2-methyl-phenylamino)-N-(prop-2-ynyloxy)-benzamide;

4-Fluoro-2-(4-iodo-2-methyl-phenylamino)-N-(2-phenoxyethoxy)-benzamide;

4-Fluoro-2-(4-iodo-2-methyl-phenylamino)-N-(2-thienylmethoxy)-benzamide;

4-Fluoro-2-(4-iodo-2-methyl-phenylamino)-N-(prop-2-enyloxy)-benzamide;

4-Fluoro-2-(4-iodo-2-methyl-phenylamino)-N-(cyclopropylmethoxy)-benzamide;

4-Fluoro-2-(4-iodo-2-methyl-phenylamino)-N-(cyclopentoxy)-benzamide;

4-Fluoro-N-hydroxy-2-(4-iodo-2-methyl-phenylamino)-N-isopropyl-benzamide; and

4-Fluoro-N-hydroxy-2-(4-iodo-2-methyl-phenylamino)-N-methyl-benzamide;

3,4-Difluoro-2-(4-iodo-2-methyl-phenylamino)-N-(3-furylmethoxy)-benzamide;

3,4-Difluoro-2-(4-iodo-2-methyl-phenylamino)-N-ethoxy-benzamide;

3,4-Difluoro-2-(4-iodo-2-methyl-phenylamino)-N-(but-2-enyloxy)-benzamide;

3,4-Difluoro-2-(4-iodo-2-methyl-phenylamino)-N-(cyclopropyl-methoxy)-benzamide;

3,4-Difluoro-2-(4-iodo-2-methyl-phenylamino)-N-(1-methylprop-2-ynyloxy)-benzamide;

3,4-Difluoro-2-(4-iodo-2-methyl-phenylamino)-N-(3-phenylprop-2-ynyloxy)-benzamide;

3,4-Difluoro-2-(4-iodo-2-methyl-phenylamino)-N-(3-methyl-5-phenylpent-2-en-4-ynyloxy)-benzamide;

3,4-Difluoro-2-(4-iodo-2-methyl-phenylamino)-N-(prop-2-ynyloxy)-benzamide;

3,4-Difluoro-2-(4-iodo-2-methyl-phenylamino)-N-(propoxy)-benzamide;

3,4-Difluoro-2-(4-iodo-2-methyl-phenylamino)-N-(cyclobutoloxy)-benzamide;

3,4-Difluoro-2-(4-iodo-2-methyl-phenylamino)-N-(2-thienylmethoxy)-benzamide;

3,4-Difluoro-2-(4-iodo-2-methyl-phenylamino)-N-(2-methyl-prop-2-enyloxy)-benzamide;

3,4-Difluoro-2-(4-iodo-2-methyl-phenylamino)-N-(2-phenoxyethoxy)-benzamide;

3,4-Difluoro-2-(4-iodo-2-methyl-phenylamino)-N-(but-2-enyloxy)-benzamide;

3,4-Difluoro-2-(4-iodo-2-methyl-phenylamino)-N-(but-3-ynyloxy)-benzamide;

3,4-Difluoro-2-(4-iodo-2-methyl-phenylamino)-N-(cyclopentyloxy)-benzamide;

3,4-Difluoro-2-(4-iodo-2-methyl-phenylamino)-N-(3-(2-fluorophenyl)-prop-2-ynyloxy)-benzamide;

3,4-Difluoro-2-(4-iodo-2-methyl-phenylamino)-N-(tetrahydro-pyran-2-yloxy)-benzamide;

3,4-Difluoro-N-hydroxy-2-(4-iodo-2-methyl-phenylamino)-benzamide;

3,4-Difluoro-2-(2-chloro-4-iodo-phenylamino)-N-cyclobutylmethoxy-benzamide;

3,4-Difluoro-2-(2-chloro-4-iodo-phenylamino)-N-(tetrahydro-pyran-2-yloxy)-benzamide; and

3,4-Difluoro-2-(2-chloro-4-iodo-phenylamino)-N-cyclopropylmethoxy-benzamide.

According the one embodiment of the present invention, the anticancer agent is a HER-1, HER-2, or HER-4 inhibitor, or a pan HER inhibitor.

In one preferred embodiment of the present invention, the anticancer agent is a pan HER inhibitor having the formula V:

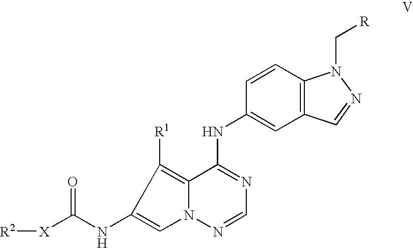

wherein

R is selected from the group consisting of aryl, substituted aryl, heterocyclo, and substituted heterocyclo;

R¹ is selected from the group consisting of alkyl and substituted alkyl;

R² is selected from the group consisting of hydrogen, alkyl, substituted alkyl, cycloalkyl, substituted cycloalkyl, aryl, substituted aryl, aralkyl, heterocyclo, and substituted heterocyclo; or, R² may be absent;

X is selected from the group consisting of a bond, O, S, C(R³)₂, C(R³)₃, NR³; and N(R³)₂;

R³ is independently selected from the group consisting of hydrogen, alkyl, substituted alkyl, aryl, substituted aryl, aralkyl, heterocyclo, and substituted heterocyclo,

and pharmaceutically acceptable salts, prodrugs, enantiomers, diastereomers, and solvates thereof.

In some preferred embodiments, the pan HER inhibitor is selected from one of the following:

[5-ethyl-4-[[(1-phenylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[(1-phenylmethyl)-1H-indazol-5-yl]amino]pyrrolo[2,1-f][1,2,4]triazin-6-yl]-carbamic acid, (2R)-2-pyrrolidinylmethyl ester,

[5-ethyl-4-[[(1-phenylmethyl)-1H-indazol-5-yl]amino]pyrrolo[2,1-f][1,2,4]triazin-6-yl]-carbamic acid, (2S)-2-pyrrolidinylmethyl ester,

[5-ethyl-4-[[(1-phenylmethyl)-1H-indazol-5-yl]amino]pyrrolo[2,1-f][1,2,4]triazin-6-yl]-carbamic acid, (3R)-3-morpholinylmethyl ester,

[5-ethyl-4-[[(1-phenylmethyl)-1H-indazol-5-yl]amino]pyrrolo[2,1-f][1,2,4]triazin-6-yl]-carbamic acid, 3-[(3S)-3-hydroxy-1-pyrrolidinyl]propyl ester,

[5-ethyl-4-[[(1-phenylmethyl)-1H-indazol-5-yl]amino]pyrrolo[2,1-f][1,2,4]triazin-6-yl]-carbamic acid, 3-[(3S)-3-hydroxy-1-piperidinyl]propyl ester,

[5-ethyl-4-[[(1-phenylmethyl)-1H-indazol-5-yl]amino]pyrrolo[2,1-f][1,2,4]triazin-6-yl]-carbamic acid, (3R)-3-pyrrolidinylmethyl ester,

[5-ethyl-4-[[(1-phenylmethyl)-1H-indazol-5-yl]amino]pyrrolo[2,1-f][1,2,4]triazin-6-yl]-carbamic acid, 3-[(3R)-3-hydroxy-1-pyrrolidinyl]propyl ester,

[5-ethyl-4-[[(1-phenylmethyl)-1H-indazol-5-yl]amino]pyrrolo[2,1-f [1,2,4]triazin-6-yl]-carbamic acid, [(2S)-1-methyl-2-pyrrolidinyl]methyl ester,

[5-ethyl-4-[[(1-phenylmethyl)-1H-indazol-5-yl]amino]pyrrolo[2,1-f][1,2,4]triazin-6-yl]-carbamic acid, (2S)-2-morpholinylmethyl ester,

[5-ethyl-4-[[(1-phenylmethyl)-1H-indazol-5-yl]amino]pyrrolo[2,1-f][1,2,4]triazin-6-yl]-carbamic acid, (3S)-3-pyrrolidinylmethyl ester,

[5-ethyl-4-[[(1-phenylmethyl)-1H-indazol-5-yl]amino]pyrrolo[2,1-f][1,2,4]triazin-6-yl]-carbamic acid, (2R)-2-morpholinylmethyl ester,

[5-ethyl-4-[[(1-phenylmethyl)-1H-indazol-5-yl]amino]pyrrolo[2,1-f][1,2,4]triazin-6-yl]-carbamic acid, [(3R)-1-methyl-3-pyrrolidinyl]methyl ester,

[5-ethyl-4-[[(1-phenylmethyl)-1H-indazol-5-yl]amino]pyrrolo[2,1-f][1,2,4]triazin-6-yl]-carbamic acid, trans-4-aminocyclohexyl ester,

[5-ethyl-4-[[(1-phenylmethyl)-1H-indazol-5-yl]amino]pyrrolo[2,1-f][1,2,4]triazin-6-yl]-carbamic acid, (3R)-3-piperidinyl ester,

[5-ethyl-4-[[(1-phenylmethyl)-1H-indazol-5-yl]amino]pyrrolo[2,1-f][1,2,4]triazin-6-yl]-carbamic acid, (3S)-3-piperidinyl ester,

[5-ethyl-4-[[(1-phenylmethyl)-1H-indazol-5-yl]amino]pyrrolo[2,1-f][1,2,4]triazin-6-yl]-carbamic acid, cis-4-aminocyclohexyl,

[5-ethyl-4-[[(1-phenylmethyl)-1H-indazol-5-yl]amino]pyrrolo[2,1-f][1,2,4]triazin-6-yl]-carbamic acid, (2R,4R)-2-(hydroxymethyl)-4-piperidinyl ester,

[5-ethyl-4-[[(1-phenylmethyl)-1H-indazol-5-yl]amino]pyrrolo[2,1-f][1,2,4]triazin-6-yl]-carbamic acid, (2S)-2-(hydroxymethyl)-4-piperidinyl ester,

[5-ethyl-4-[[(1-phenylmethyl)-1H-indazol-5-yl]amino]pyrrolo[2,1-f][1,2,4]triazin-6-yl]-carbamic acid, cis-4-(aminomethyl)cyclohexyl ester,

[5-ethyl-4-[[(1-phenylmethyl)-1H-indazol-5-yl]amino]pyrrolo[2,1-f][1,2,4]triazin-6-yl]-carbamic acid, cis-4-amino-4-methylcyclohexyl ester,

[5-ethyl-4-[[(1-phenylmethyl)-1H-indazol-5-yl]amino]pyrrolo[2,1-f][1,2,4]triazin-6-yl]-carbamic acid, [(2R,4R)-4-(hydroxy-2-piperidinyl]methylester,

[5-ethyl-4-[[(1-phenylmethyl)-1H-indazol-5-yl]amino]pyrrolo[2,1-f][1,2,4]triazin-6-yl]-carbamic acid, trans-4-(aminomethyl)cyclohexyl ester,

[5-ethyl-4-[[1-(2-oxazolylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[1-(2-thienylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[1-[(3-fluorophenyl)methyl]-1H-indazol-5-yl]amino]pyrrolo[2,1-f][1,2,4]triazin-6-yl]-carbamic acid, (3S)-3-morpholinylmethyl ester,

[5-ethyl-4-[[1-(4-thiazolylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[1-(3-thienylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[1-(2-pyridinylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[1-(2-thiazolylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[1-(3-pyridinylmethyl)-1H-indazol-5-yl]amino]pyrrolo[2,1-f][1,2,4]triazin-6-yl]-carbamic acid, (3S)-3-morpholinylmethyl ester,

[5-ethyl-4-[[1-(pyrazinylmethyl)-1H-indazol-5-yl]amino]pyrrolo[2,1-f][1,2,4]triazin-6-yl]-carbamic acid, (3S)-3-morpholinylmethyl ester,

[4-[[1-(3-fluorophenyl)methyl]-1H-indazol-5-ylamino]-5-methyl-pyrrolo[2,1-f][1,2,4]triazin-6-yl]-carbamic acid, trans-4-aminocyclohexyl ester,

[4-[[1-(3-fluorophenyl)methyl]-1H-indazol-5-ylamino]-5-methyl-pyrrolo [2,1-f][1,2,4]triazin-6-yl]-carbamic acid, (2R,4R)-2-(hydroxymethyl)-4-piperidinyl ester,

[4-[[1-(3-fluorophenyl)methyl]-1H-indazol-5-ylamino]-5-methyl-pyrrolo[2,1-f][1,2,4]triazin-6-yl]-carbamic acid, (2S,4S)-2-(hydroxymethyl)-4-piperidinyl ester,

[4-[[1-(3-fluorophenyl)methyl]-1H-indazol-5-ylamino]-5-methyl-pyrrolo[2,1-f][1,2,4]triazin-6-yl]-carbamic acid, cis-4-aminocyclohexyl ester,

[4-[[1-(3-fluorophenyl)methyl]-1H-indazol-5-ylamino]-5-methyl-pyrrolo[2,1-f][1,2,4]triazin-6-yl]-carbamic acid, cis-4-amino-4-methyl-cyclohexyl ester,

[4-[[1-(3-fluorophenyl)methyl]-1H-indazol-5-ylamino]-5-methyl-pyrrolo[2,1-f][1,2,4]triazin-6-yl]-carbamic acid, (2R)-2-aminopropyl ester,

[4-[[1-(3-fluorophenyl)methyl]-1H-indazol-5-ylamino]-5-methyl-pyrrolo[2,1-f][1,2,4]triazin-6-yl]-carbamic acid, (2S)-2-aminopropyl ester,

[4-[[1-(3-fluorophenyl)methyl]-1H-indazol-5-ylamino]-5-methyl-pyrrolo[2,1-f][1,2,4]triazin-6-yl]-carbamic acid, (3S)-3-morpholinylmethyl ester,

[4-[[1-(3-fluorophenyl)methyl]-1H-indazol-5-ylamino]-5-methyl-pyrrolo[2,1-f][1,2,4]triazin-6-yl]-carbamic acid, (3R)-3-piperidinyl ester,

[4-[[1-(3-fluorophenyl)methyl]-1H-indazol-5-ylamino]-5-methyl-pyrrolo[2,1-f][1,2,4]triazin-6-yl]-carbamic acid, (3S)-3-piperidinyl ester,

3-[[[[[4-[[1-[(3-fluorophenyl)methyl]-1H-indazol-5-yl]amino]-5-methylpyrrolo[2,1-f][1,2,4]triazin-6-yl]amino]carbonyl]oxy]methyl]-4-morpholinecarboxylic acid, (3S)-1,1-dimethylethyl ester,

[4-[[1-(3-fluorophenyl)methyl]-1H-indazol-5-ylamino]-5-methyl-pyrrolo[2,1-f][1,2,4]triazin-6-yl]-carbamic acid, 3-morpholinylmethyl ester, and

[4-[[1-(3-fluorophenyl)methyl]-1H-indazol-5-ylamino]-5-methyl-pyrrolo[2,1-f][1,2,4]triazin-6-yl]-carbamic acid, (3R)-3-morpholinylmethyl ester.

According to one embodiment of the present invention, the pan HER inhibitor has the formula VI:

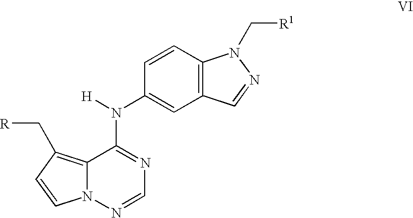

and includes its enantiomers, diastereomers, and pharmaceutically acceptable salts, prodrugs and solvates thereof, wherein

R is selected from the group consisting of SR², SOR², SO₂R², OR², and NR³R⁴;

R¹ is selected from the group consisting of aryl, substituted aryl, heterocyclo, and substituted heterocyclo;

R² is selected from the group consisting of hydrogen, alkyl, substituted alkyl, aryl, substituted aryl, aralkyl, heterocyclo, and substituted heterocyclo;

R³ and R⁴ are independently selected from the group consisting of hydrogen, alkyl,

substituted alkyl, aryl, substituted aryl, heterocyclo, and substituted heterocyclo;

or R² and R³ may together form an optionally substituted monocyclic 4-8 membered saturated or unsaturated carbocyclic or heterocyclic ring, or an optionally substituted bicyclic 7 to 12 membered saturated or unsaturated carbocyclic or heterocyclic ring.

According to one embodiment of the present invention, the anticancer agent is a cytotoxic agent. Cytotoxic agents include, without limitation, the following:

Alkylating agents (including, without limitation, nitrogen mustards, ethylenimine derivatives, alkyl sulfonates, nitrosoureas and triazenes): Uracil mustard, Chlormethine, Cyclophosphamide (Cytoxan®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as Taxol®), Mithramycin, Deoxyco-formycin, Mitomycin-C, L-Asparaginase, Interferons (especially IFN-a), Etoposide, and Teniposide.

Other anti-proliferative cytotoxic agents are navelbene, CPT-11, anastrazole, letrazole, capecitabine, reloxafine, cyclophosphamide, ifosamide, and droloxafine.

Microtubule affecting agents interfere with cellular mitosis and are well known in the art for their anti-proliferative cytotoxic activity. Microtubule affecting agents useful in the invention include, but are not limited to, allocolchicine (NSC 406042), Halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in the scientific and patent literature, see, e.g., Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; Panda (1996) J. Biol. Chem 271:29807-29812.

Paclitaxel is a preferred anticancer agent of the present invention. Paclitaxel inhibits eukaryotic cell replication by enhancing polymerization of tubulin moieties into stabilized microtubule bundles that are unable to reorganize into the proper structures for mitosis. Of the many available chemotherapeutic drugs, paclitaxel has generated interest because of its efficacy in clinical trials against drug-refractory tumors, including ovarian and mammary gland tumors (Hawkins (1992) Oncology, 6: 17-23, Horwitz (1992) Trends Pharmacol. Sci. 13: 134-146, Rowinsky (1990) J. Natl. Canc. Inst. 82: 1247-1259).

In some embodiments of the present invention, the cytotoxic agent has paclitaxel-like activity. These include, but are not limited to, paclitaxel and paclitaxel derivatives (paclitaxel-like compounds) and analogues. Paclitaxel and its derivatives are available commercially. In addition, methods of making paclitaxel and paclitaxel derivatives and analogues are well known to those of skill in the art (see, e.g., U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506).

Thus, anti-proliferative cytotoxic agents which are suitable for use in the methods and compositions of this invention include, but are not limited to, microtubule-stabilizing agents such as paclitaxel (also known as Taxol®), docetaxel (also known as Taxotere®), 7-O-methylthiomethylpaclitaxel (disclosed in U.S. Pat. No. 5,646,176), 4-desacetyl-4-methylcarbonatepaclitaxel, 3′-tert-butyl-3′-N-tert-butyloxycarbonyl-4-deacetyl-3′-dephenyl-3′-N-debenzoyl-4-O-methoxycarbonyl-paclitaxel (disclosed in U.S. Ser. No. 09/712,352 filed on Nov. 14, 2000), C-4 methyl carbonate paclitaxel, epothilone A, epothilone B, epothilone C, epothilone D, desoxyepothilone A, desoxyepothilone B, [1S-[1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17 oxabicyclo[14.1.0]heptadecane-5,9-dione (disclosed in WO 99/02514), [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Pat. No. 6,262,094) and derivatives thereof; and microtubule-disruptor agents.

Also suitable are cytotoxic agents such as CDK inhibitors, an antiproliferative cell cycle inhibitor, epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Additional cytotoxic agents include, melphalan, hexamethyl melamine, thiotepa, cytarabin, idatrexate, trimetrexate, dacarbazine, L-asparaginase, camptothecin, topotecan, bicalutamide, flutamide, leuprolide, pyridobenzoindole derivatives, interferons, and interleukins.

The present invention also encompasses a pharmaceutical composition useful in the treatment of cancer, comprising a therapeutically effective amount of the combinations of this invention and may comprise an additional anti-cancer agent or agents, and a pharmaceutically acceptable carrier. The compositions of the present invention may further comprise one or more pharmaceutically acceptable additional ingredient(s) such as alum, stabilizers, antimicrobial agents, buffers, coloring agents, flavoring agents, adjuvants, and the like.

The present invention also encompasses a pharmaceutical composition useful in the treatment of cancer, comprising a therapeutically effective amount of the combinations of this invention and may comprise an additional anti-cancer agent or agents, and a pharmaceutically acceptable carrier. The compositions of the present invention may further comprise one or more pharmaceutically acceptable additional ingredient(s) such as alum, stabilizers, antimicrobial agents, buffers, coloring agents, flavoring agents, adjuvants, and the like.

The IGF1R and anticancer agents of the present invention are administered orally or parenterally including the intravenous, intramuscular, intraperitoneal, subcutaneous, rectal and topical routes of administration.

For oral use, compositions of this invention may be administered, for example, in the form of tablets or capsules, powders, dispersible granules, or cachets, or as aqueous solutions or suspensions. In the case of tablets for oral use, carriers that are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added. In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations that are intended for conversion, shortly before use, to liquid preparations for either oral or parenteral administration. Such liquid forms include solutions, suspensions and emulsions.

The IGF1R and/or anticancer agents of the present invention can be delivered transdermally. The transdermal compositions can take the form of creams, lotions, aerosols and/or emulsions and can be included in a transdermal patch of the matrix or reservoir type as are conventional in the art for this purpose.

The IGF1R inhibitor may be administered prior to, simultaneously with, or subsequent to the administration of the anticancer agent.

The combinations of the present invention may also be used in conjunction with other well-known anticancer therapies, including radiation, chemotherapy and surgery. Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, USA); the disclosure of which is incorporated herein by reference thereto.

The actual dosage employed may be varied depending upon the requirements of the patient and the severity of the condition being treated. Generally, treatment is initiated with smaller dosages that are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

Also, in general, the IGF1R inhibitors and the anticancer agents do not have to be administered in the same pharmaceutical composition, and may, because of different physical and chemical characteristics, have to be administered by different routes. For example, the IGF1R inhibitor may be administered orally to generate and maintain good blood levels thereof, while the anticancer agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of IGF1R inhibitor and anticancer agent will depend upon the diagnosis of the attending physicians and their judgment of the condition of the patient and the appropriate treatment protocol.

Administration of either the IGF1R inhibitor and/or anticancer agent can be repeated during a single treatment protocol. The determination of the order of administration, and the number of repetitions of administration of each therapeutic agent during a treatment protocol, is well within the knowledge of the skilled physician after evaluation of the disease being treated and the condition of the patient.

Thus, in accordance with experience and knowledge, the practicing physician can modify each protocol for the administration of a component of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

In order to facilitate a further understanding of the invention, the following examples are presented primarily for the purpose of illustrating more specific details thereof. The scope of the invention should not be deemed limited by the examples, but encompasses the entire subject matter defined in the claims.

### EXAMPLES

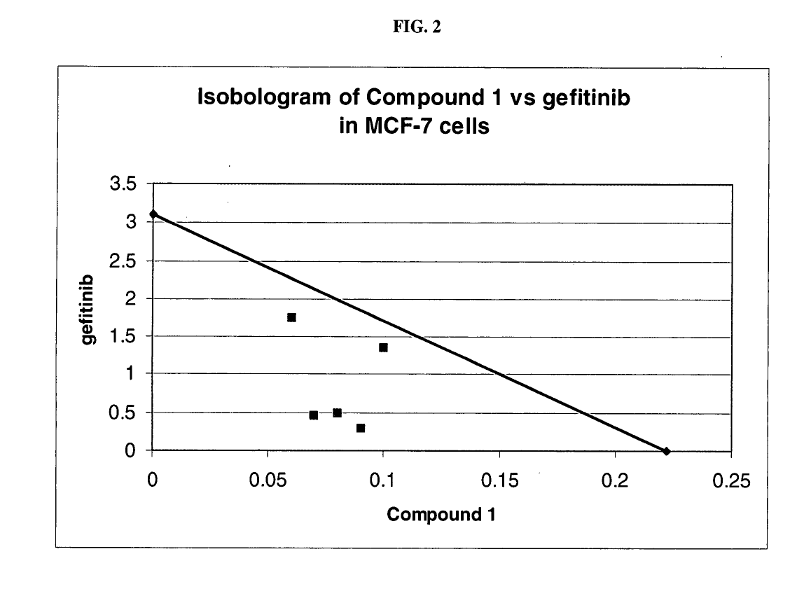

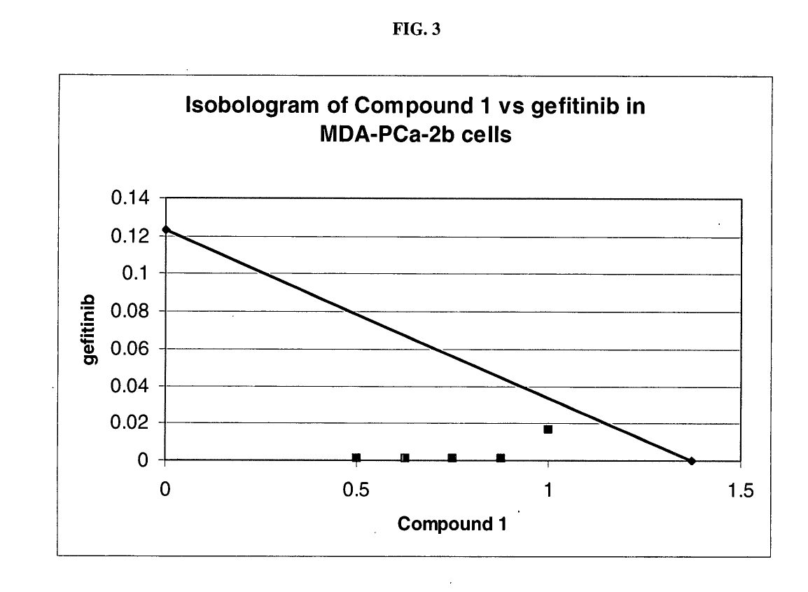

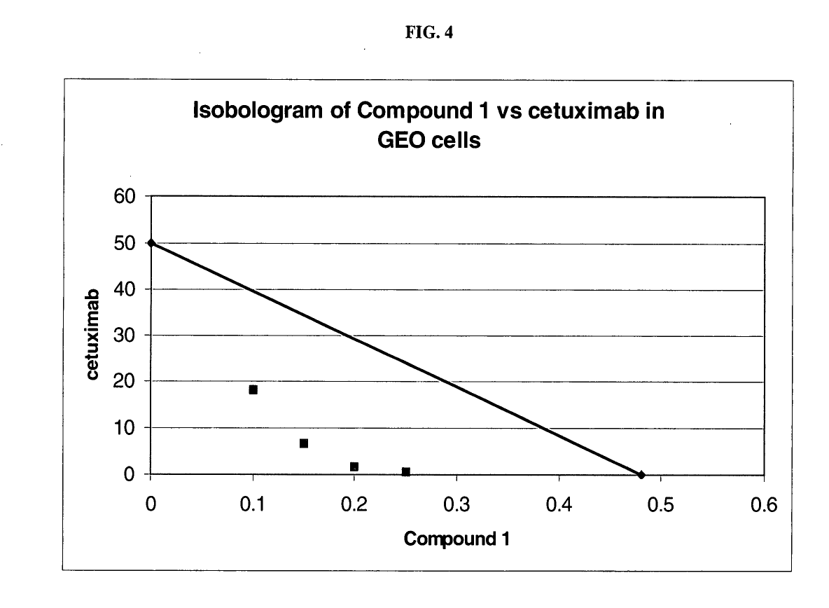

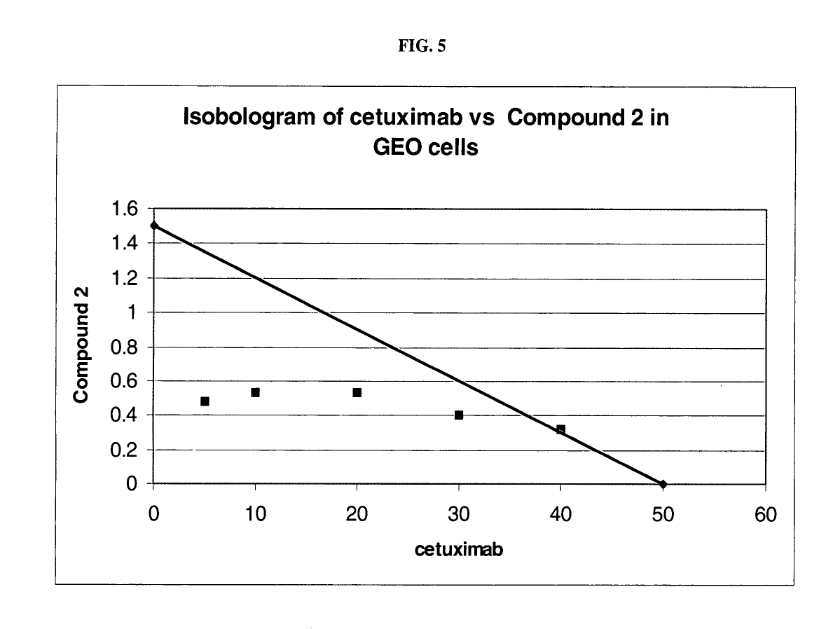

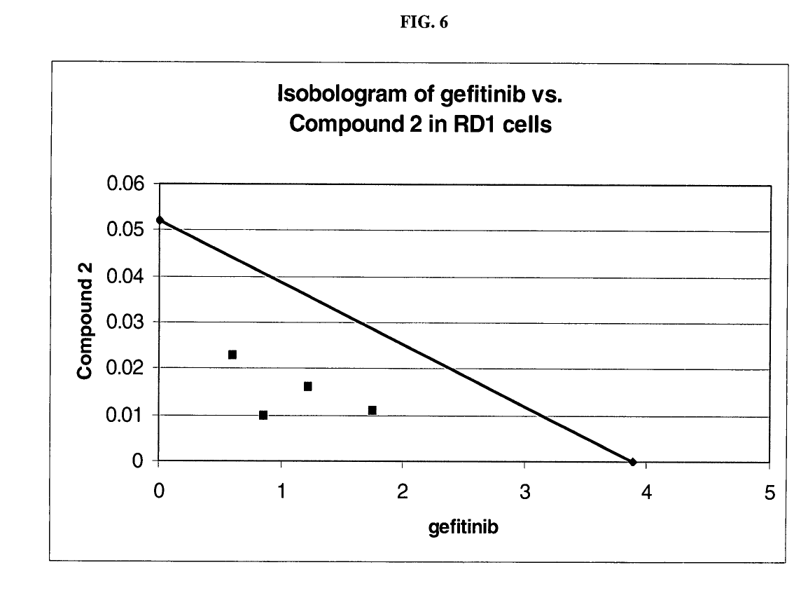

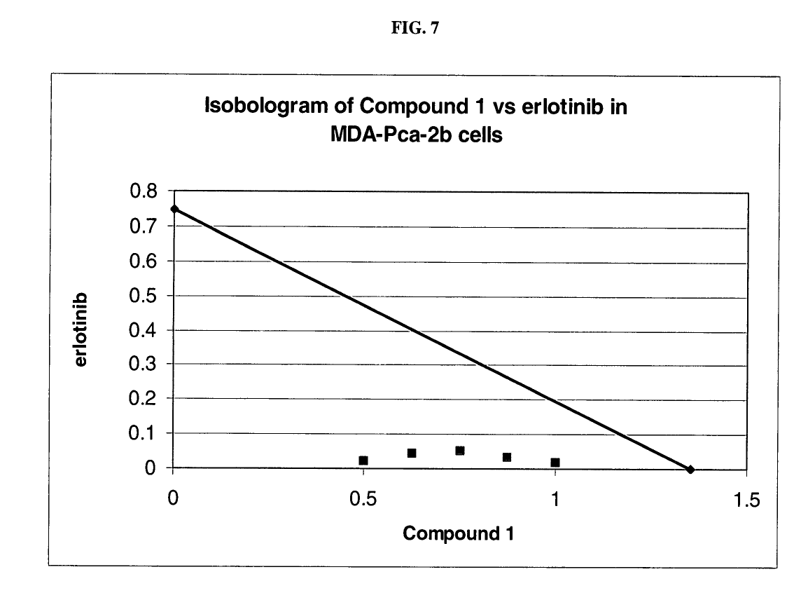

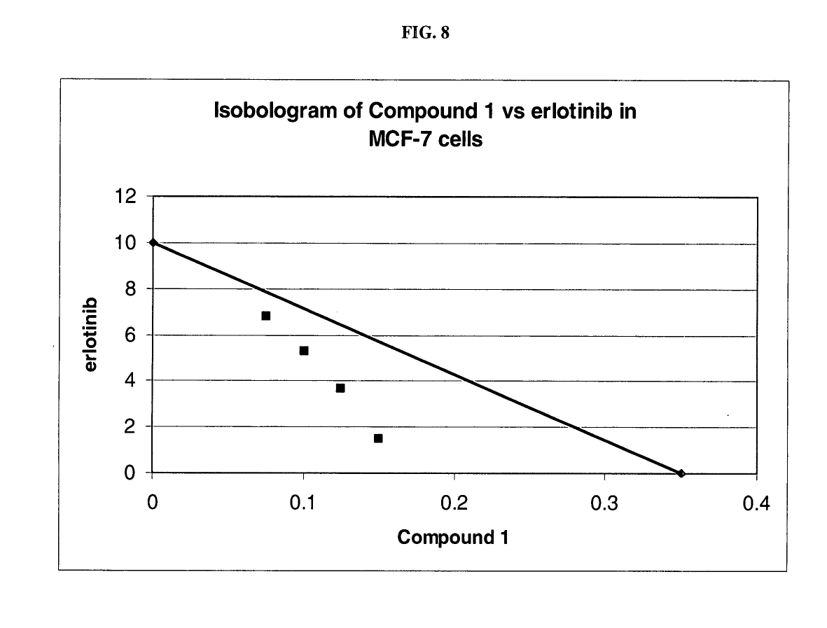

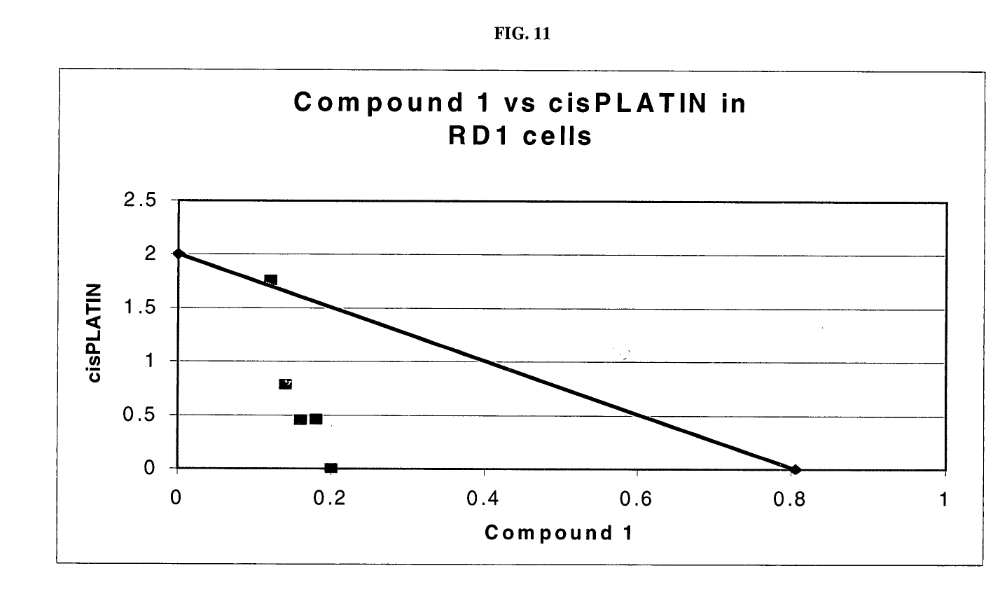

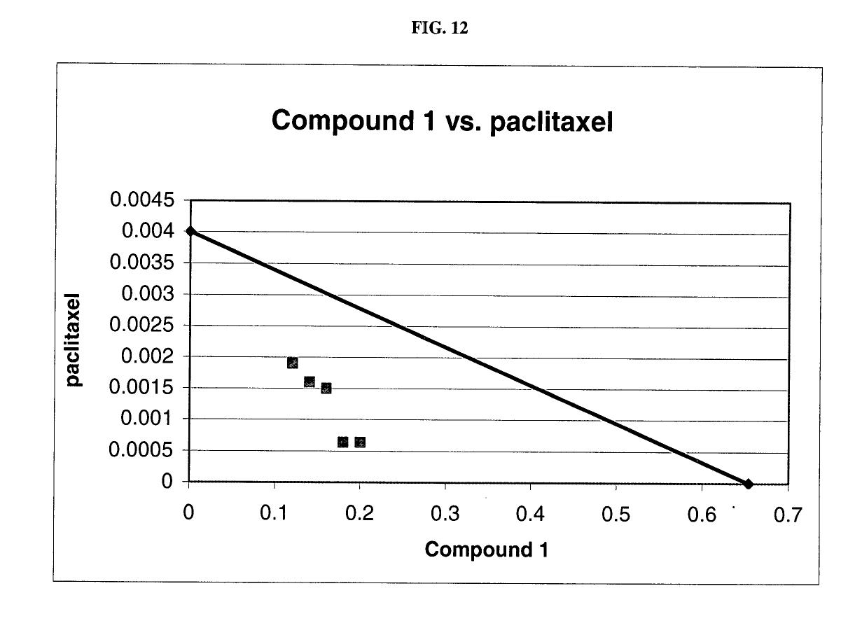

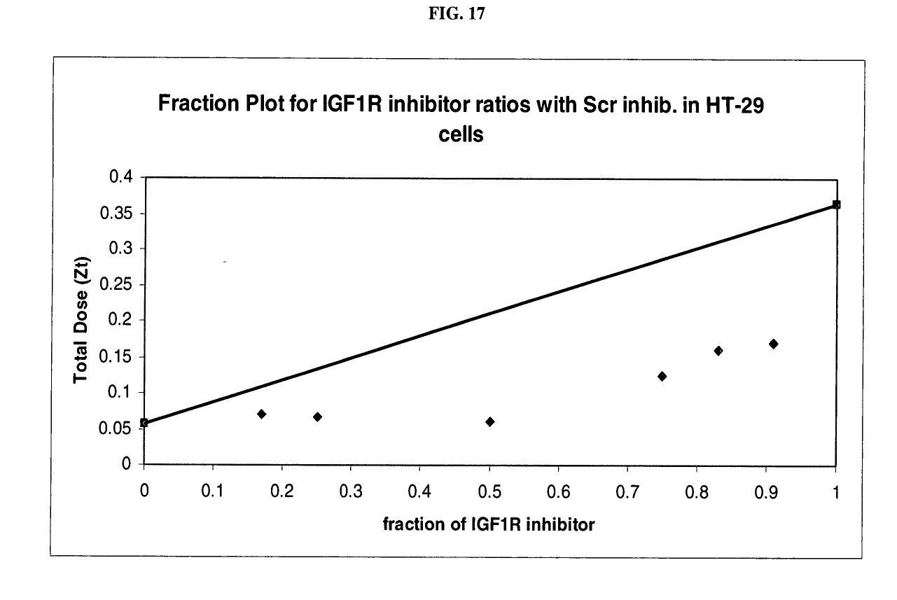

FIGS. 1 through 17 demonstrate the synergistic effects achieved when an IGF1R inhibitor of Formula I is administered in combination with an additional anticancer agent. Isobolograms and fraction plots are used to analyze the data.

### Example 1

### ³H-Thymidine Uptake Cell Proliferation Assay Utilizing Drug Combinations of IGF1R Inhibitors and Additional Anticancer Agents

Stock drug concentrations were 10 mM in 100% DMSO (dimethyl sulfoxide), with subsequent dilutions performed in 70% DMSO.

Serial dilutions (1:4 or 1:5) were used to establish the 50% inhibitory dose of both the test and standard compounds alone. The cells were seeded in a 50 ul volume using a 96-well format 24 hrs prior to addition of the drug. The next day, each well received an additional 25 ul of the test compound or media (containing DMSO), and 25 ul of the standard compound or media (containing DMSO). A dose response curve was established for the standard compound; the test compound was then added as a single dose to the standard compound dose curves. All wells contain a final volume of 100 ul and a final concentration of 0.35% DMSO.

After dosing, the cells were allowed to incubate at 37° C. in an atmosphere of 5% CO₂ until they were labeled with 0.44 uCi/well ³H-thymidine; after a total of 72 hours post dosing, wells were harvested. Wells without cells were used to calculate a background value, and wells with cells but without drug were used to calculate a total control value. At harvest, the cells were trypsized and the amount of ³H-thymidine incorporated was captured by glass filter and counted by scintillation.

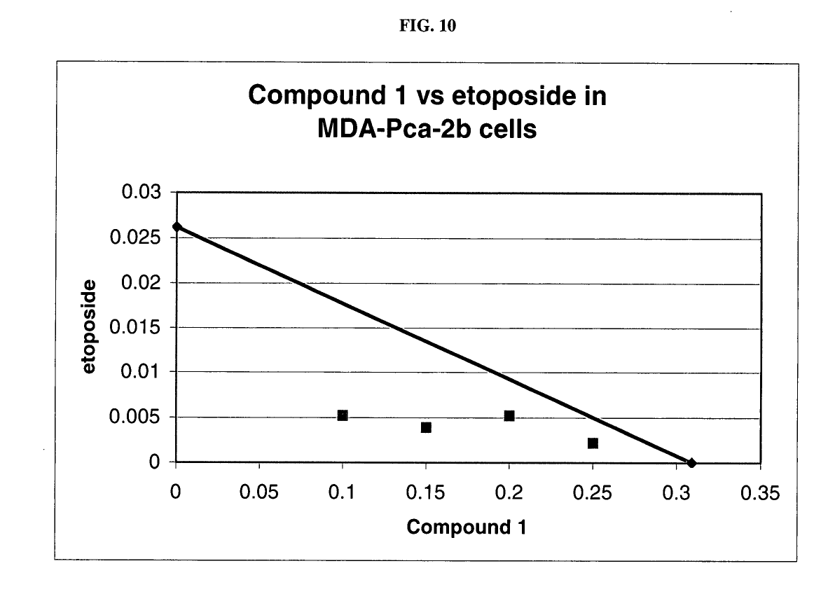

Concentrations of each drug alone or combinations of the two drugs administered together that blocked growth by 50% (IC₅₀) were calculated. Assuming zero interaction between the two compounds, these points on the axes can be joined by a straight line (isobole) which indicates combinations of standard and test drugs that are isoeffective with either drug alone. The isoeffect is the IC₅₀. When drug combinations fall along this straight line they are assumed to be additive. When the drug combinations are more effective than expected, lower concentrations are required to produce the isoeffect (IC₅₀) and are considered synergistic. These points will fall below the zero interaction isobole. When drug combinations require higher concentrations than expected to produce the isoeffect, they are considered antagonistic and the points will fall above the zero interaction isobole. All of the combinations tested fall at or below the zero interaction isobole as depicted in FIGS. 10, 11, and 12 “Compound 1” represents an IGF1R inhibitor according to Formula I as described above.

### Example 2

### ³H-Thymidine Uptake Cell Proliferation Assay Utilizing Drug Combinations of IGF1R Inhibitors and EGFR Inhibitors

Stock drug concentrations were 10 mM in 100% DMSO (dimethyl sulfoxide), with subsequent dilutions performed in 70% DMSO.

Serial dilutions (1:4 or 1:5) were used to establish the 50% inhibitory dose of both the test and standard compounds alone. The cells were seeded in a 50 ul volume using a 96-well format 24 hrs prior to addition of the drug. The next day, each well received an additional 25 ul of the test compound or media (containing DMSO), and 25 ul of the standard compound or media (containing DMSO). A dose response curve was established for the standard compound; the test compound was then added as a single dose to the standard compound dose curves. All wells contain a final volume of 100 ul and a final concentration of 0.35% DMSO.

After dosing, the cells were allowed to incubate at 37° C. in an atmosphere of 5% CO₂ until they were labeled with 0.44 uCi/well ³H-thymidine; after a total of 72 hours post dosing, wells were harvested. Wells without cells were used to calculate a background value, and wells with cells but without drug were used to calculate a total control value. At harvest, the cells were trypsized and the amount of ³H-thymidine incorporated was captured by glass filter and counted by scintillation.

Concentrations of each drug alone or combinations of the two drugs administered together that blocked growth by 50% (IC₅₀) were calculated. Assuming zero interaction between the two compounds, these points on the axes can be joined by a straight line (isobole) which indicates combinations of standard and test drugs that are isoeffective with either drug alone. The isoeffect is the IC₅₀. When drug combinations fall along this straight line they are assumed to be additive. When the drug combinations are more effective than expected, lower concentrations are required to produce the isoeffect (IC₅₀) and are considered synergistic. These points will fall below the zero interaction isobole. When drug combinations require higher concentrations than expected to produce the isoeffect, they are considered antagonistic and the points will fall above the zero interaction isobole. All of the combinations tested fall at or below the zero interaction isobole as depicted in FIGS. 1 through 8 wherein “Compound 1” and “Compound 2” represent IGF1R inhibitors according to Formula I.

### Example 3

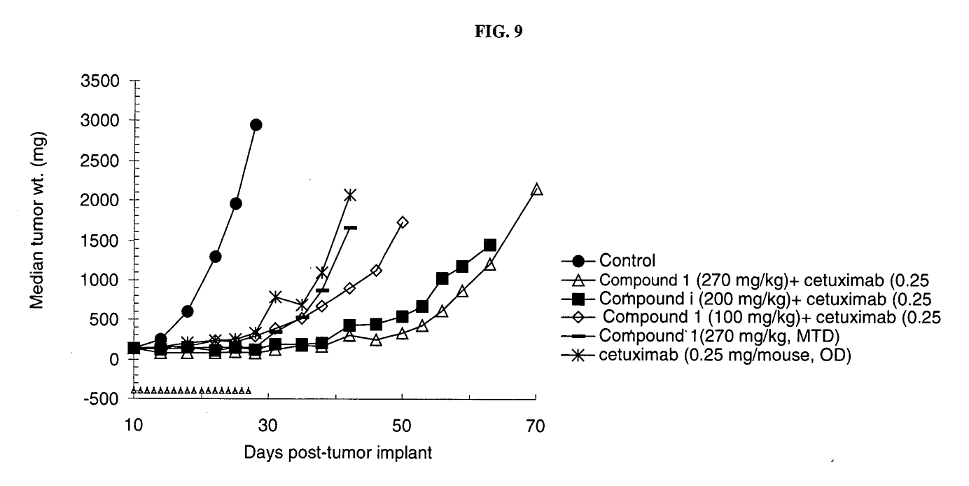

Chemotherapy trials were conducted with an IGF1R inhibitor (Compound 1) and an EGFR inhibitor (cetuximab), either singly or in combination, in nude mice bearing advanced-stage GEO human colon carcinoma xenografts. As monotherapy, both agents demonstrated significant antitumor activities, inhibiting tumor growth/progression and causing significant tumor growth delay (TGD, delay of tumor progression to a predetermined tumor burden). Treatment of mice with Compound 1 at its MTD of 270 mg/kg/adm, po, qdx17 yielded TGD value of 18.5 days. Cetuximab at its optimal dose of 0.25 mg/mouse, ip, q3dx6, produced TGD of 14.5 days. However, when used in combination the two agents produced antitumor efficacies that were far superior than those that could be produced maximally by either single agent alone (i.e., at their MTD or OD). Thus, using the maximally tolerated regimen (270 mg/kg/adm Compound 1 plus 0.25 mg/mouse cetuximab) the combination produced a TGD of 40.3 days, significantly better than single agent Compound 1 (P=0.0009) or single agent cetuximab (P=0.0008). Even more significant, superior antitumor efficacies were obtained with combination regimens that were below the maximally tolerated level and thus effectively improving the efficacy/tolerability margin of therapeutic strategies that target EGFR and IGF1R for the treatment of cancer. FIG. 9 depicts the effects of Compound 1 and cetuximab treatment, singly and in combination, on the growth of the GEO human colon carcinoma xenograft model in nude mice.

Importantly, in this study several combination regimens of Compound 1 and cetuximab, even at dose levels that are clearly below the MTD level, produced antitumor efficacies that were significantly superior than the optimal efficacy obtained with either single agent alone (at their respective MTD or OD), thus satisfying the definition of therapeutic synergism. On the other hand, the combination of Compound 1 and cetuximab produced toxicity that was no greater than either single agent alone, in terms of both weight loss and mortality.

The present invention is not limited to the embodiments specifically described above, but is capable of variation and modification without departure from the scope of the appended claims.

### Example 4

### ³H-Thymidine Uptake Cell Proliferation Assay Utilizing Drug Combinations of IGF1R Inhibitors and an Inhibitor of MEK

Stock drug concentrations were 10 mM in 100% DMSO (dimethyl sulfoxide), with subsequent dilutions performed in 70% DMSO.

Serial dilutions (1:4 or 1:5) were used to establish the 50% inhibitory dose of both the test and standard compounds alone. The IGF1R-sal cells were seeded (at 1500/well) in a 50 ul volume using a 96-well format 24 hrs prior to addition of the drug. The next day, each well received an additional 25 ul of the test compound or media (containing DMSO), and 25 ul of the standard compound or media (containing DMSO). A dose response curve was established for the standard compound; the test compound was then added as a single dose to the standard compound dose curves. All wells contain a final volume of 100 ul and a final concentration of 0.35% DMSO.

After dosing, the cells were allowed to incubate at 37° C. in an atmosphere of 5% CO₂ until they were labeled with 0.44 uCi/well ³H-thymidine; after a total of 72 hours post dosing, wells were harvested. Wells without cells were used to calculate a background value, and wells with cells but without drug were used to calculate a total control value. At harvest, the cells were trypsized and the amount of ³H-thymidine incorporated was captured by glass filter and counted by scintillation.

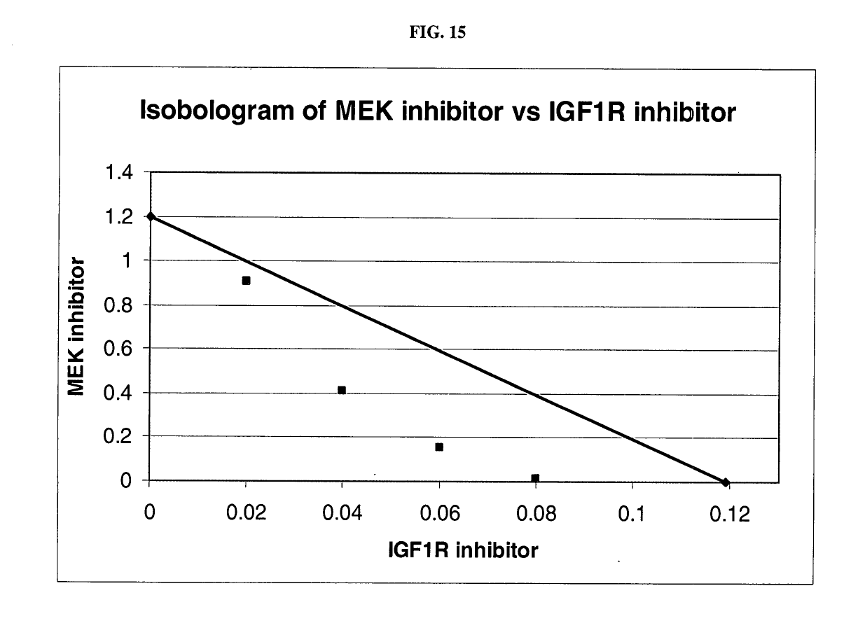

Concentrations of each drug alone or combinations of the two drugs administered together that blocked growth by 50% (IC₅₀) were calculated. Assuming zero interaction between the two compounds, these points on the axes can be joined by a straight line (isobole) which indicates combinations of standard and test drugs that are isoeffective with either drug alone. The isoeffect is the IC₅₀. When drug combinations fall along this straight line they are assumed to be additive. When the drug combinations are more effective than expected, lower concentrations are required to produce the isoeffect (IC₅₀) and are considered synergistic. These points will fall below the zero interaction isobole. When drug combinations require higher concentrations than expected to produce the isoeffect, they are considered antagonistic and the points will fall above the zero interaction isobole. All of the combinations tested fall at or below the zero interaction isobole as depicted in FIG. 15 wherein “Compound 1” represents an IGF1R inhibitor according to Formula I as described above.

### Example 5

### ³H-Thymidine Uptake Cell Proliferation Assay Utilizing a Dilution of Ratios Drug Combination Method of IGF1R Inhibitors and Pan Her Inhibitors

Drug Stocks (of 10 mM concentration in DMSO) for two compounds, were combined in ratios of 10:1, 5:1, 3:1, 1:1, 1:3, 1:5. These ratios, as well as the individual compound stock solutions, were diluted in a serial manner, using 70% DMSO. Typically the dose curves were started taking into account the dilution factors needed to achieve the final concentration. For the highest concentration of 1 uM final, the initial concentration is 200 uM. Two ul of this solution was transferred to 198 ul of complete RPMI tissue culture media (1/100 dilution=2 uM). Finally 50 ul of this media is added to a 50 ul culture in a 96 well plate (a ½ dilution=1 uM final concentration), containing cells which were plated the previous day. Colo205 at 5000 cells/well was used in one example. After addition of the drug, these cultures were allowed to incubate at 37° C. with 5% CO₂ for 72 hrs, including the final 3 hrs with 0.44 uCi/well of ³H-Thymidine. The cells were trypsinized and harvested onto glass filters which were counted using scintillation. The IC₅₀ values were estimated using the amounts of incorporated ³H-Thymidine as compared to untreated cells, with all samples in triplicate wells.

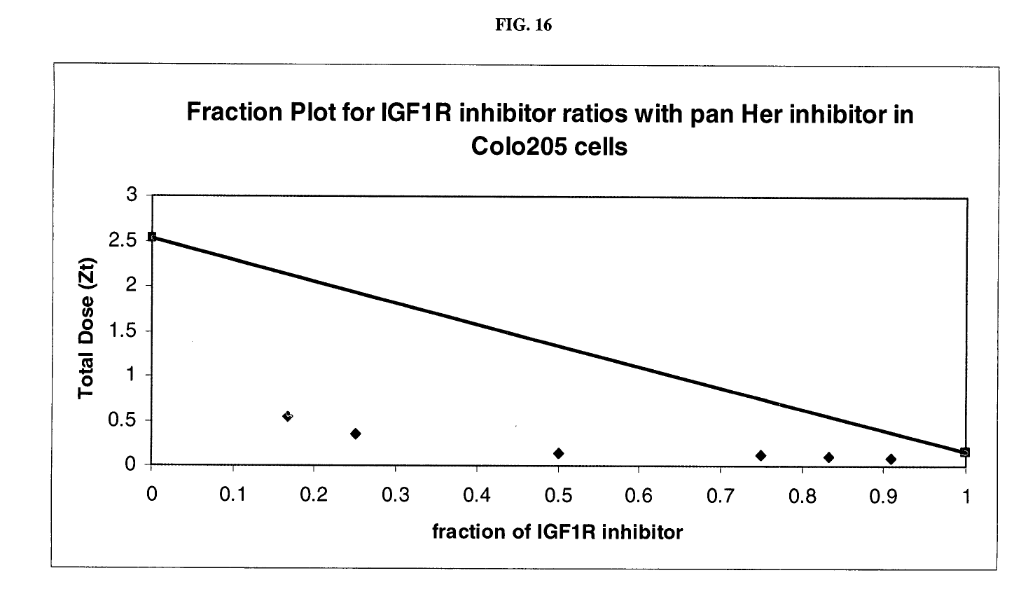

The total dose of the two compounds were used to calculate the IC₅₀ values for the dose curves. A fractions graph was used to generate an isobologram, where the values for the two compounds alone, at either end of the graph, were connected by a trend line. The IC₅₀ values for the combinations, were added at their fraction value (on the x-axis). Combination IC₅₀ values which graph below the trend line are considered to be synergistic, and values which fall close to the line are evidence of an additive effect, as shown in FIG. 16.

Statistical analysis of the Combination Indexes can include using the standard errors for the IC₅₀ values (calculated for the individual compounds) and the IC50 results for the separate combination ratios, to calculate a Combination Index value with a 95% confidence interval for each ratio. Combination index values below a value of 1 was considered to be synergistic when the 95% confidence interval (index value+/−2× standard error) did not exceed the value of 1.

### Example 6

### ³H-Thymidine Uptake Cell Proliferation Assay Utilizing a Dilution of Ratios Drug Combination Method of IGF1R Inhibitors and Src Inhibitors

Drug Stocks (of 10 mM concentration in DMSO) for two compounds, are combined in ratios of 10:1, 5:1, 3:1, 1:1, 1:3, 1:5. These ratios, as well as the individual compound stock solutions, were diluted in a serial manner, using 70% DMSO. Typically the dose curves will be started taking into account the dilution factors needed to achieve the final concentration. For the highest concentration of 1 uM final, the initial concentration was 200 uM. Two ul of this solution was transferred to 198 ul of complete RPMI tissue culture media (1/100 dilution=2 uM). Finally 50 ul of this media was added to a 50 ul culture in a 96 well plate (a ½ dilution=1 uM final concentration), containing cells which were plated the previous day. In two of runs, HT29 at 5000 cells/well or Colo205 at 5000 cells/well were used. After addition of the drug, these cultures were allowed to incubate at 37° C. with 5% CO₂ for 72 hrs, including the final 3 hrs with 0.44 uCi/well of ³H-Thymidine. The cells were trypsinized and harvested onto glass filters which were counted using scintillation. The IC₅₀ values were estimated using the amounts of incorporated ³H-Thymidine as compared to untreated cells, with all samples in triplicate wells.

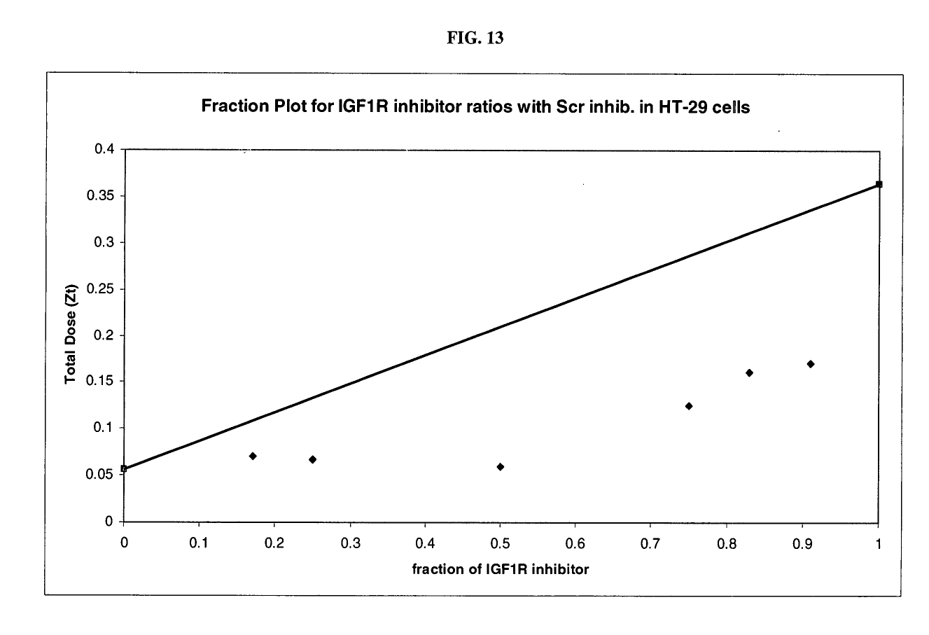

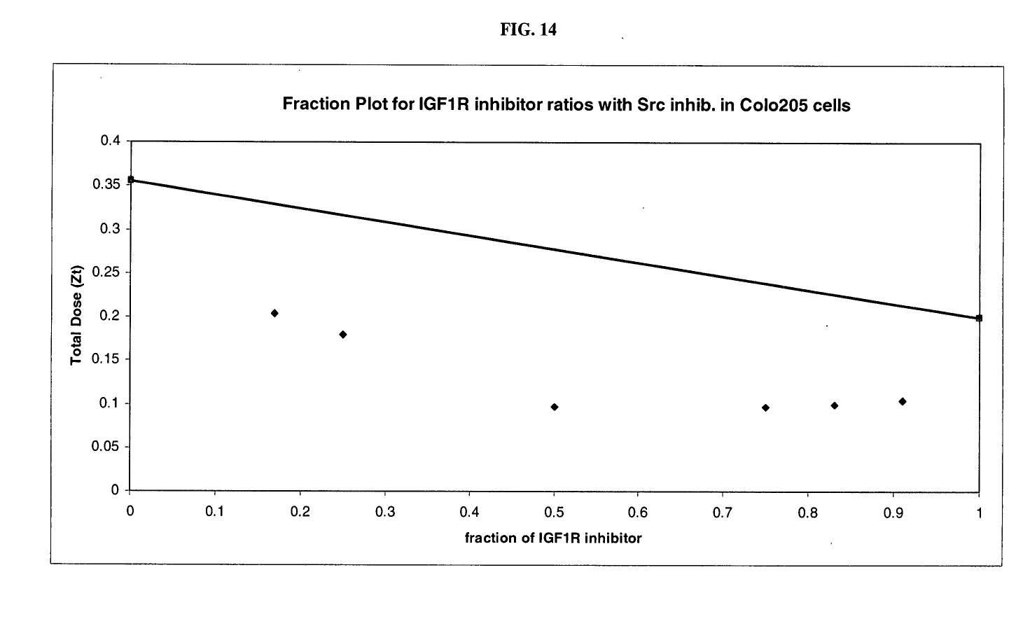

The total dose of the two compounds was used to calculate the IC₅₀ values for the dose curves. A fractions graph was used to generate an isobologram, where the values for the two compounds alone, at either end of the graph, were connected by a trend line. The IC₅₀ values for the combinations, were added at their fraction value (on the x-axis). Combination IC₅₀ values which graph below the trend line are considered to be synergistic, and values which fall close to the line are evidence of an additive effect, as shown in FIGS. 13 and 14.

Statistical analysis of the Combination Indexes can include using the standard errors for the IC₅₀ values (calculated for the individual compounds) and the IC50 results for the separate combination ratios, to calculate a Combination Index value with a 95% confidence interval for each ratio. Combination index values below a value of 1 was considered to be synergistic when the 95% confidence interval (index value+/−2× standard error) did not exceed the value of 1.

We claim:

1. A method for the synergistic treatment of cancer comprising administering to a mammal in need thereof a therapeutically effective amount of an IGF1R inhibitor in combination with a therapeutically effective amount of an additional anticancer agent in amounts sufficient to achieve a synergistic result.

2. The method according to claim 1 wherein said anticancer agent is a pan HER inhibitor, a Src inhibitor, a MEK inhibitor, or an EGFR inhibitor.

3. The method according to claim 2 wherein the anticancer agent is an EGFR inhibitor.

4. The method according to claim 3 wherein said EGFR inhibitor is erlotinib.

5. The method according to claim 3 wherein said EGFR inhibitor is cetuximab.

6. The method according to claim 3 wherein said EGFR inhibitor is gefitinib.

7. The method according to claim 2 wherein said anticancer agent is a Src inhibitor.

8. The method according to claim 7 wherein the Src inhibitor is selected from the group consisting of:
[5-[[(2,4,6-Trimethylphenyl)amino]carbonyl]-4-methyl-2-thiazolyl]carbamic acid, 1,1-dimethylethyl ester;
[5-[[(2,4,6-Trimethylphenyl)amino]carbonyl]-4-trifluoromethyl-2-thiazolyl]carbamic acid, 1,1-dimethylethyl ester;
2-Amino-N-(2,4,6-trimethylphenyl)-4-trifluoromethyl-5-thiazolecarboxamide, trifluoroacetate (1:1);
[5-[[(2,4,6-Trimethylphenyl)amino]carbonyl]-4-phenyl-2-thiazolyl]carbamic acid, 1,1-dimethylethyl ester;
2-Amino-N-(2,4,6-trimethylphenyl)-4-phenyl-5-thiazolecarboxamide, trifluoroacetate (1:1);
[5-[[phenylamino]carbonyl]-4-methyl-2-thiazolyl]carbamic acid, 1,1-dimethylethyl ester;
[5-[[(2,4-Dichlorophenyl)amino]carbonyl]-4-methyl-2-thiazolyl]carbamic acid, 1,1-dimethylethyl ester;
5-[[(2,4,6-Trimethylphenyl)amino]carbonyl]-2-thiazolyl]carbamic acid, 1,1-dimethylethyl ester;
2-Amino-N-(2,4,6-trimethylphenyl)-4-phenyl-5-thiazolecarboxamide, trifluoroacetate (1:1);
[5-[[(2-Methoxy-6-methylphenyl)amino]carbonyl]-4-methyl-2-thiazolyl]carbamic acid 1,1-dimethylethyl ester;
[4-Methyl-5-[[[3-methyl-4-(1-methylethyl)phenyl]amino]carbonyl]-2-thiazolyl]carbamic acid 1,1-dimethylethyl ester;
[5-[[(4-Bromo-2,6-dimethylphenyl)amino]carbonyl]-4-methyl-2-thiazolyl]carbamic acid 1,1-dimethylethyl ester;
[4-Methyl-5-[[[2-methyl-6-(1-methylethyl)phenyl]amino]carbonyl]-2-thiazolyl]carbamic acid 1,1-dimethylethyl ester;
[5-[[(2,4-Dimethylphenyl)amino]carbonyl]-4-methyl-2-thiazolyl]carbamic acid 1,1-dimethylethyl ester;
[4-Methyl-5-[[(2-methylphenyl)amino]carbonyl]-2-thiazolyl]carbamic acid 1,1-dimethylethyl ester;
[5-[[(2-Chloro-6-methylphenyl)amino]carbonyl]-4-methyl-2-thiazolyl]carbamic acid 1,1-dimethylethyl ester;
[5-[[[2-(1,1-Dimethylethyl)-4-methylphenyl]amino]carbonyl]-4-methyl-2-thiazolyl]carbamic acid 1,1-dimethylethyl ester;
[5-[[(2-Furanylmethyl)amino]carbonyl]-4-methyl-2-thiazolyl]carbamic acid 1,1-dimethylethyl ester;
[5-[[[3-Methoxy-5-(trifluoromethyl)phenyl]amino]carbonyl]-4-methyl-2-thiazolyl]carbamic acid 1,1-dimethylethyl ester;
[5-[[(4-Cyclohexylphenyl)amino]carbonyl]-4-methyl-2-thiazolyl]carbamic acid 1,1-dimethylethyl ester;
[5-[[(Cyclohexylmethyl)amino]carbonyl]-4-methyl-2-thiazolyl]carbamic acid 1,1-dimethylethyl ester;
[5-[[(2,3-Dihydro-1H-indenyl)amino]carbonyl]-4-methyl-2-thiazolyl]carbamic acid 1,1-dimethylethyl ester;
[5-[(2,5-Dihydro-1H-pyrrol-1-yl)carbonyl]-4-methyl-2-thiazolyl]carbamic acid 1,1-dimethylethyl ester;
[5-[(2,5-Dihydro-2,5-dimethyl-1H-pyrrol-1-yl)carbonyl]-4-methyl-2-thiazolyl]carbamic acid 1,1-dimethylethyl ester;
1-[[2-[[(1,1-Dimethylethoxy)carbonyl]amino]-4-methyl-5-thiazolyl]carbonyl]-L-prolinamide;
[5-[(4-Formyl-1-piperazinyl)carbonyl]-4-methyl-2-thiazolyl]carbamic acid 1,1-dimethylethyl ester;
[5-(1,4-Dioxa-8-azaspiro[4.5]decan-8-ylcarbonyl)-4-methyl-2-thiazolyl]carbamic acid 1,1-dimethylethyl ester;
[5-[[3-[(Diethylamino)carbonyl]-1-piperidinyl]carbonyl]-4-methyl-2-thiazolyl]carbamic acid 1,1-dimethylethyl ester;
[4-Methyl-5-[(octahydro-1-quinolinyl)carbonyl]-2-thiazolyl]carbamic acid 1,1-dimethylethyl ester;
2-[[(1,1-Dimethylethoxy)carbonyl]amino]-4-methyl-5-thiazolecarboxylic acid 2-[(1,1-dimethylethoxy)carbonyl]hydrazide;
[5-[[(4-Methoxyphenyl)amino]carbonyl]-4-methyl-2-thiazolyl]carbamic acid 1,1-dimethylethyl ester;
[4-Methyl-5-[[(4-methylphenyl)amino]carbonyl]-2-thiazolyl]carbamic acid 1,1-dimethylethyl ester;
[5-[[(1,2-Dimethylpropyl)amino]carbonyl]-4-methyl-2-thiazolyl]carbamic acid 1,1-dimethylethyl ester;
[5-[[(2,2-Dimethylpropyl)amino]carbonyl]-4-methyl-2-thiazolyl]carbamic acid 1,1-dimethylethyl ester;
[4-Methyl-5-[(2-propynylamino)carbonyl]-2-thiazolyl]carbamic acid 1,1-dimethylethyl ester;
[4-Methyl-5-[(2-propenylamino)carbonyl]-2-thiazolyl]carbamic acid 1,1-dimethylethyl ester;
[4-Methyl-5-[(methylphenylamino)carbonyl]-2-thiazolyl]carbamic acid 1,1-dimethylethyl ester;
[4-Methyl-5-[[(3,4,5-trimethoxyphenyl)amino]carbonyl]-2-thiazolyl]carbamic acid 1,1-dimethylethyl ester;
[5-[[[2,6-Bis(1-methylethyl)phenyl]amino]carbonyl]-4-methyl-2-thiazolyl]carbamic acid 1,1-dimethylethyl ester;
[5-[[[3-(1H-Imidazol-1-yl)propyl]amino]carbonyl]-4-methyl-2-thiazolyl]carbamic acid 1,1-dimethylethyl ester;
[5-[[[(3,4-Difluorophenyl)methyl]amino]carbonyl]-4-methyl-2-thiazolyl]carbamic acid 1,1-dimethylethyl ester;
N-[[2-[[(1,1-Dimethylethoxy)carbonyl]amino]-4-methyl-5-thiazolyl]carbonyl]-L-leucine methyl ester;
5-[[[2-[[(1,1-Dimethylethoxy)carbonyl]amino]-4-methyl-5-thiazolyl]carbonyl]amino]-4-oxopentanoic acid methyl ester;
[5-[[[2-(Ethylthio)ethyl]amino]carbonyl]-4-methyl-2-thiazolyl]carbamic acid 1,1-dimethylethyl ester;
[5-[[Bis(3-methylbutyl)amino]carbonyl]-4-methyl-2-thiazolyl]carbamic acid 1,1-dimethylethyl ester;
[5-[[Ethyl(1-methylethyl)amino]carbonyl]-4-methyl-2-thiazolyl]carbamic acid 1,1-dimethylethyl ester;
2-[[(1,1-Dimethylethoxy)carbonyl]amino]-4-methyl-5-thiazolecarboxylic acid 2-[[(3,5-dichlorophenyl)amino]thioxomethyl]hydrazide;
[5-[[Bis(2-ethoxyethyl)amino]carbonyl]-4-methyl-2-thiazolyl]carbamic acid 1,1-dimethylethyl ester;
[4-Methyl-5-[[3-[(trifluoroacetyl)amino]-1-pyrrolidinyl]carbonyl]-2-thiazolyl]carbamic acid 1,1-dimethylethyl ester;
[5-[[(2,6-Dimethylphenyl)amino]carbonyl]-4-methyl-2-thiazolyl]carbamic acid 1,1-dimethylethyl ester;
2-[[(2,2-Dichloro-1-methylcyclopropyl)carbonyl]amino]-4-methyl-N-(2,4,6-trimethylphenyl)-5-thiazolecarboxamide;
2-[(Cyclohexylacetyl)amino]-4-methyl-N-(2,4,6-trimethylphenyl)-5-thiazolecarboxamide;
2-[(2,5-Difluorobenzoyl)amino]-4-methyl-N-(2,4,6-trimethylphenyl)-5-thiazolecarboxamide;
2-[(5-Bromo-2-chlorobenzoyl)amino]-4-methyl-N-(2,4,6-trimethylphenyl)-5-thiazolecarboxamide;
2-[(3-Cyanobenzoyl)amino]-4-methyl-N-(2,4,6-trimethylphenyl)-5-thiazolecarboxamide;
2-[[4-(Acetylamino)benzoyl]amino]-4-methyl-N-(2,4,6-trimethylphenyl)-5-thiazolecarboxamide;
4-Methyl-2-[[3-(trifluoromethyl)benzoyl]amino]-N-(2,4,6-trimethylphenyl)-5-thiazolecarboxamide;
4-Methyl-2-[[2-(2-phenylethyl)benzoyl]amino]-N-(2,4,6-trimethylphenyl)-5-thiazolecarboxamide;
2-[(3,5-Dimethylbenzoyl)amino]-4-methyl-N-(2,4,6-trimethylphenyl)-5-thiazolecarboxamide;
2-[(4-Ethenylbenzoyl)amino]-4-methyl-N-(2,4,6-trimethylphenyl)-5-thiazolecarboxamide;
2-[(4-Butylbenzoyl)amino]-4-methyl-N-(2,4,6-trimethylphenyl)-5-thiazolecarboxamide;
4-Methyl-2-[(4-pentylbenzoyl)amino]-N-(2,4,6-trimethylphenyl)-5-thiazolecarboxamide;
4-Methyl-2-[(1-oxo-3-phenoxypropyl)amino]-N-(2,4,6-trimethylphenyl)-5-thiazolecarboxamide;
4-Methyl-2-[(1-oxo-3-phenylpropyl)amino]-N-(2,4,6-trimethylphenyl)-5-thiazolecarboxamide;
2-[[3-(2-Methoxyphenyl)-1-oxopropyl]amino]-4-methyl-N-(2,4,6-trimethylphenyl)-5-thiazolecarboxamide;
4-Methyl-2-[(2-naphthalenylacetyl)amino]-N-(2,4,6-trimethylphenyl)-5-thiazolecarboxamide;
2-[(Diphenylacetyl)amino]-4-methyl-N-(2,4,6-trimethylphenyl)-5-thiazolecarboxamide;
2-[[(2-Chloro-6-fluorophenyl)acetyl]amino]-4-methyl-N-(2,4,6-trimethylphenyl)-5-thiazolecarboxamide;
4-Methyl-2-[[(2-methylphenyl)acetyl]amino]-N-(2,4,6-trimethylphenyl)-5-thiazolecarboxamide;
2-[[(3-Methoxyphenyl)acetyl]amino]-4-methyl-N-(2,4,6-trimethylphenyl)-5-thiazolecarboxamide;
2-[[(3,4-Dimethoxyphenyl)acetyl]amino]-4-methyl-N-(2,4,6-trimethylphenyl)-5-thiazolecarboxamide;
2-[[(4-Chlorophenyl)acetyl]amino]-4-methyl-N-(2,4,6-trimethylphenyl)-5-thiazolecarboxamide;
2-[([1,1′-Biphenyl]-4-ylacetyl)amino]-4-methyl-N-(2,4,6-trimethylphenyl)-5-thiazolecarboxamide;
4-Methyl-2-[(1-oxo-4-phenylbutyl)amino]-N-(2,4,6-trimethylphenyl)-5-thiazolecarboxamide;
2-[(2-Hydroxy-2-phenyl-1-oxopropyl)amino]-4-methyl-N-(2,4,6-trimethylphenyl)-5-thiazolecarboxamide;
2-[(2-Hydroxy-1-oxohexyl)amino]-4-methyl-N-(2,4,6-trimethylphenyl)-5-thiazolecarboxamide;
4-Methyl-2-[[1-oxo-4-(2-thienyl)butyl]amino]-N-(2,4,6-trimethylphenyl)-5-thiazolecarboxamide;
4-Methyl-2-[(3-thienylcarbonyl)amino]-N-(2,4,6-trimethylphenyl)-5-thiazolecarboxamide;
2-[(2-Benzofuranylcarbonyl)amino]-4-methyl-N-(2,4,6-trimethylphenyl)-5-thiazolecarboxamide;
N-[4-Methyl-5-[[(2,4,6-trimethylphenyl)amino]carbonyl]-2-thiazolyl]-4-pyridinecarboxamide, N-oxide;
6-Chloro-N-[4-methyl-5-[[(2,4,6-trimethylphenyl)amino]carbonyl]-2-thiazolyl]-3-pyridinecarboxamide;
N-[4-Methyl-5-[[(2,4,6-trimethylphenyl)amino]carbonyl]-2-thiazolyl]-3-pyridinecarboxamide;
N-[4-Methyl-5-[[(2,4,6-trimethylphenyl)amino]carbonyl]-2-thiazolyl]-3-quinolinecarboxamide;
4-Methyl-2-[[(4-nitrophenyl)acetyl]amino]-N-(2,4,6-trimethylphenyl)-5-thiazolecarboxamide;
4-Methyl-2-[(2,4,6-trichlorobenzoyl)amino]-N-(2,4,6-trimethylphenyl)-5-thiazolecarboxamide;
4-Methyl-2-[[2-[[3-(trifluoromethyl)phenyl]amino]benzoyl]amino]-N-(2,4,6-trimethylphenyl)-5-thiazolecarboxamide;
4-Methyl-2-[[4-(4-nitrophenyl)-1-oxobutyl]amino]-N-(2,4,6-trimethylphenyl)-5-thiazolecarboxamide;
4-Methyl-2-[[4-(methylsulfonyl)benzoyl]amino]-N-(2,4,6-trimethylphenyl)-5-thiazolecarboxamide;
2-[(4-Heptylbenzoyl)amino]-4-methyl-N-(2,4,6-trimethylphenyl)-5-thiazolecarboxamide;
2-[[(2,4-Difluorophenyl)acetyl]amino]-4-methyl-N-(2,4,6-trimethylphenyl)-5-thiazolecarboxamide;
(S)-2-[[2-(Dipropylamino)-1-oxopropyl]amino]-4-methyl-N-(2,4,6-trimethylphenyl)-5-thiazolecarboxamide;
2-[(2-Biphenylenecarbonyl)amino]-4-methyl-N-(2,4,6-trimethylphenyl)-5-thiazolecarboxamide;
2-[[3-(3-Methoxyphenyl)-1-oxopropyl]amino]-4-methyl-N-(2,4,6-trimethylphenyl)-5-thiazolecarboxamide;
4-Methyl-N-(2,4,6-trimethylphenyl)-2-[[(2,4,6-trimethylphenyl)acetyl]amino]-5-thiazolecarboxamide;
4-Methyl-2-[(1-oxo-6-heptenyl)amino]-N-(2,4,6-trimethylphenyl)-5-thiazolecarboxamide;
2-[[(1,3-Benzodioxol-5-yl)acetyl]amino]-4-methyl-N-(2,4,6-trimethylphenyl)-5-thiazolecarboxamide;
4-Methyl-2-[[[2-(phenylmethoxy)phenyl]acetyl]amino]-N-(2,4,6-trimethylphenyl)-5-thiazolecarboxamide;
4-Methyl-2-[[(3-phenoxyphenyl)acetyl]amino]-N-(2,4,6-trimethylphenyl)-5-thiazolecarboxamide;
2-[(3,5-Dimethoxyphenyl)acetyl]amino]-4-methyl-N-(2,4,6-trimethylphenyl)-5-thiazolecarboxamide;
2-[[4-[4-[Bis(2-chloroethyl)amino]phenyl]-1-oxobutyl]amino]-4-methyl-N-(2,4,6-trimethylphenyl)-5-thiazolecarboxamide;
4-[[4-[[[4-methyl-5-[[(2,4,6-trimethylphenyl)amino]carbonyl]-2-thiazolyl]amino]carbonyl]phenyl]amino]-4-oxobutanoic acid methyl ester;
4-Methyl-2-[[(phenylsulfonyl)acetyl]amino]-N-(2,4,6-trimethylphenyl)-5-thiazolecarboxamide;
2-[[2-(Acetylamino)-1-oxohexyl]amino]-4-methyl-N-(2,4,6-trimethylphenyl)-5-thiazolecarboxamide;
2-[[4-[(Dipropylamino)sulfonyl]benzoyl]amino]-4-methyl-N-(2,4,6-trimethylphenyl)-5-thiazolecarboxamide;
2-[(4-Cyclohexylbenzoyl)amino]-4-methyl-N-(2,4,6-trimethylphenyl)-5-thiazolecarboxamide;
2-[(4-Bromo-3-methylbenzoyl)amino]-4-methyl-N-(2,4,6-trimethylphenyl)-5-thiazolecarboxamide;
2-[[(2,3-Difluorophenyl)acetyl]amino]-4-methyl-N-(2,4,6-trimethylphenyl)-5-thiazolecarboxamide;
4-Methyl-2-[[[4-(1-methylethyl)phenyl]acetyl]amino]-N-(2,4,6-trimethylphenyl)-5-thiazolecarboxamide;
2-[[[4-(1,1-Dimethylethyl)cyclohexyl]carbonyl]amino]-4-methyl-N-(2,4,6-trimethylphenyl)-5-thiazolecarboxamide;
N,N-Dimethyl-N′-[4-methyl-5-[[(2,4,6-trimethylphenyl)amino]carbonyl]-2-thiazolyl]butanediamide;
2-[(1,6-Dioxohexyl)amino]-4-methyl-N-(2,4,6-trimethylphenyl)-5-thiazolecarboxamide;
2-[(Benzo[b]thiophen-2-ylcarbonyl)amino]-4-methyl-N-(2,4,6-trimethylphenyl)-5-thiazolecarboxamide;
2-[(1-Adamantylcarbonyl)amino]-4-methyl-N-(2,4,6-trimethylphenyl)-5-thiazolecarboxamide;
4-Methyl-2-[[(4-methylcyclohexyl)carbonyl]amino]-N-(2,4,6-trimethylphenyl)-5-thiazolecarboxamide;
2-[(1,7-Dioxooctyl)amino]-4-methyl-N-(2,4,6-trimethylphenyl)-5-thiazolecarboxamide;
2-[[2-(Acetylamino)-4-(ethylthio)-1-oxobutyl]amino]-4-methyl-N-(2,4,6-trimethylphenyl)-5-thiazolecarboxamide;
1,5-Dimethyl-N-[4-methyl-5-[[(2,4,6-trimethylphenyl)amino]carbonyl]-2-thiazolyl]-1H-pyrazole-3-carboxamide;
2-[[[4-methyl-5-[[(2,4,6-trimethylphenyl)amino]carbonyl]-2-thiazolyl]amino]carbonyl]benzoic acid;
N-[4-Methyl-5-[[(2,4,6-trimethylphenyl)amino]carbonyl]-2-thiazolyl]-6-benzothiazolecarboxamide;
1-Ethyl-4-methyl-N-[4-methyl-5-[[(2,4,6-trimethylphenyl)amino]carbonyl]-2-thiazolyl]-1H-pyrazole-3-carboxamide;
4-Methyl-2-[[3-[(3H-1,2,3-triazolo[4,5-b]pyridin-3-yloxy)methyl]benzoyl]amino]-N-(2,4,6-trimethylphenyl)-5-thiazolecarboxamide;
2-[(2-Furanylcarbonyl)amino]-4-methyl-N-(2,4,6-trimethylphenyl)-5-thiazolecarboxamide;
2-[(4-Chlorobenzoyl)amino]-4-methyl-N-(2,4,6-trimethylphenyl)-5-thiazolecarboxamide;
[4-Methyl-5[[(2-nitrophenyl)amino]carbonyl]-2-thiazolyl]carbamic acid, 1,1-dimethylethyl ester;
[4-Methyl-5 [[(2,4,6-trimethylphenyl)amino]carbonyl]-2-thiazolyl]carbamic acid, phenylmethyl ester;
Methyl [4-methyl-5-[[(2,4,6-trimethylphenyl)amino]carbonyl]-2-thiazolyl]carbamic acid, 1,1-dimethylethyl ester;
[4-Methyl-5-[[methyl(2,4,6-trimethylphenyl)amino]carbonyl]-2-thiazolyl]carbamic acid, 1,1-dimethylethyl ester;
[4-Methyl-5 [[(2,4,6-trimethylphenyl)amino]carbonyl]-2-thiazolyl]carbamic acid, methyl ester;
[4-Ethyl-5 [[(2,4,6-trimethylphenyl)amino]carbonyl]-2-thiazolyl]carbamic acid, 1,1-dimethylethyl ester;
[5-[[(2,6-Dichlorophenyl)amino]carbonyl]-4-methyl-2-thiazolyl]carbamic acid, 1,1-dimethylethyl ester;
2-Amino-N-(2-methyl-6-isopropylphenyl)-4-methyl-5-thiazolecarboxamide, trifluoroacetate (1:1);
2-(Benzoylamino)-4-methyl-N-(2,4,6-trimethylphenyl)-5-thiazolecarboxamide;
4-Methyl-2-[(phenylcetyl)amino]-N-(2,4,6-trimethylphenyl)-5-thiazolecarboxamide;
2-[[(Acetylamino)acetyl]amino]-4-methyl-N-(2,4,6-trimethylphenyl)-6-thiazolecarboxamide;
2-Amino-4-methyl-N-(2,4,6-trimethylphenyl)-5-thiazolecarbothioamide;
2-[(4-Bromobenzoyl)amino]-4-methyl-N-(2,4,6-trimethylphenyl)-5-thiazolecarboxamide;
4-Methyl-2-[(4-nitrobenzoyl)amino]-N-(2,4,6-trimethylphenyl)-5-thiazolecarboxamide;
2-[(4-Cyanobenzoyl)amino]-4-methyl-N-(2,4,6-trimethylphenyl)-5-thiazolecarboxamide;
4-Methyl-2-[[(5-nitro-2-furanyl)carbonyl]amino]-N-(2,4,6-trimethylphenyl)-5-thiazolecarboxamide;
4-Methyl-2-[(2-thienylcarbonyl)amino]-N-(2,4,6-trimethylphenyl)-5-thiazolecarboxamide;
4-[[[4-Methyl-5-[[(2,4,6-trimethylphenyl)amino]carbonyl]-2-thiazolyl]amino]carbonyl]benzoic acid methyl ester;
2-[(5-Isoxazolylcarbonyl)amino]-4-methyl-N-(2,4,6-trimethylphenyl)-5-thiazolecarboxamide;
2-[(3-Furanylcarbonyl)amino]-4-methyl-N-(2,4,6-trimethylphenyl)-5-thiazolecarboxamide;
2-[[(2,4-Dimethyl-5-thiazolyl)carbonyl]amino]-4-methyl-N-(2,4,6-trimethylphenyl)-5-thiazolecarboxamide;
2-[[(4-Methoxy-3-thienyl)carbonyl]amino]-4-methyl-N-(2,4,6-trimethylphenyl)-5-thiazolecarboxamide;
4-Methyl-2-[[(5-nitro-3-thienyl)carbonyl]amino]-N-(2,4,6-trimethylphenyl)-5-thiazolecarboxamide;
2-[[[4-[(4-Chlorophenyl)thio]-3-thienyl]carbonyl]amino]-4-methyl-N-(2,4,6-trimethylphenyl)-5-thiazolecarboxamide;
2-[[(5-Chloro-4-methoxy-3-thienyl)carbonyl]amino]-4-methyl-N-(2,4,6-trimethylphenyl)-5-thiazolecarboxamide;
2-[[[2-(4,5-Dihydro-4,4-dimethyl-2-oxazolyl)-3-thienyl]carbonyl]amino]-4-methyl-N-(2,4,6-trimethylphenyl)-5-thiazolecarboxamide;
2-[[(2-Acetyl-3-thienyl)carbonyl]amino]-4-methyl-N-(2,4,6-trimethylphenyl)-5-thiazolecarboxamide;
4-Methyl-2-[[(methylamino)carbonyl]amino]-N-(2,4,6-trimethylphenyl)-5-thiazolecarboxamide;
4-Methyl-2-[[(phenylamino)carbonyl]amino]-N-(2,4,6-trimethylphenyl)-5-thiazolecarboxamide;
4-Methyl-2-[[[(4-methylphenyl)amino]carbonyl]amino]-N-(2,4,6-trimethylphenyl)-5-thiazolecarboxamide;
4-Methyl-2-[[[(phenylmethyl)amino]carbonyl]amino]-N-(2,4,6-trimethylphenyl)-5-thiazolecarboxamide;
2-[[(Butylamino)carbonyl]amino]-4-methyl-N-(2,4,6-trimethylphenyl)-5-thiazolecarboxamide;
4-Methyl-2-[[(propylamino)carbonyl]amino]-N-(2,4,6-trimethylphenyl)-5-thiazolecarboxamide;
2-[[(Cyclohexylamino)carbonyl]amino]-4-methyl-N-(2,4,6-trimethylphenyl)-5-thiazolecarboxamide;
2-[[[(2-Chlorophenyl)amino]carbonyl]amino]-4-methyl-N-(2,4,6-trimethylphenyl)-5-thiazolecarboxamide;
2-[[[(3-Fluorophenyl)amino]carbonyl]amino]-4-methyl-N-(2,4,6-trimethylphenyl)-5-thiazolecarboxamide;
2-[[[(2,6-Dimethylphenyl)amino]carbonyl]amino]-4-methyl-N-(2,4,6-trimethylphenyl)-5-thiazolecarboxamide;
[5-[[(2,4,6-Trimethylphenyl)amino]carbonyl]-4-methyl-2-thiazolyl]carbamic acid, phenyl ester;
4-Methyl-2-[[[(2-phenylethyl)amino]carbonyl]amino]-N-(2,4,6-trimethylphenyl)-5-thiazolecarboxamide
2-[[(Hexylamino)carbonyl]amino]-4-methyl-N-(2,4,6-trimethylphenyl)-5-thiazolecarboxamide;
2-[[[(1,1-Dimethylethyl)amino]carbonyl]amino]-4-methyl-N-(2,4,6-trimethylphenyl)-5-thiazolecarboxamide;
2-[[[(3-Fluoro-4-methylphenyl)amino]carbonyl]amino]-4-methyl-N-(2,4,6-trimethylphenyl)-5-thiazolecarboxamide;
2-[[[(4-Methoxyphenyl)amino]carbonyl]amino]-4-methyl-N-(2,4,6-trimethylphenyl)-5-thiazolecarboxamide;
2-[[(Diethylamino)carbonyl]amino]-4-methyl-N-(2,4,6-trimethylphenyl)-5-thiazolecarboxamide;
2-[[[Bis(1-methylethyl)amino]carbonyl]amino]-4-methyl-N-(2,4,6-trimethylphenyl)-5-thiazolecarboxamide;
4-Methyl-2-[[[methyl(phenylmethyl)amino]carbonyl]amino]-N-(2,4,6-trimethylphenyl)-5-thiazolecarboxamide;
4-Methyl-2-[[(methylphenylamino)carbonyl]amino]-N-(2,4,6-trimethylphenyl)-5-thiazolecarboxamide;
2-[[(Cyclohexylmethylamino)carbonyl]amino]-4-methyl-N-(2,4,6-trimethylphenyl)-5-thiazolecarboxamide;
4-Methyl-2-[[[(1-phenylethyl)amino]carbonyl]amino]-N-(2,4,6-trimethylphenyl)-5-thiazolecarboxamide;
2-[[[(Cyclopropylmethyl)propylamino]carbonyl]amino]-4-methyl-N-(2,4,6-trimethylphenyl)-5-thiazolecarboxamide;
4-Methyl-2-[[[(2-methylcyclohexyl)amino]carbonyl]amino]-N-(2,4,6-trimethylphenyl)-5-thiazolecarboxamide;
4-Methyl-2-[[[(4-methylcyclohexyl)amino]carbonyl]amino]-N-(2,4,6-trimethylphenyl)-5-thiazolecarboxamide;
2-[[[(Cyclohexylmethyl)amino]carbonyl]amino]-4-methyl-N-(2,4,6-trimethylphenyl)-5-thiazolecarboxamide;
2-[[[(2,3-Dihydro-1H-inden-1-yl)amino]carbonyl]amino]-4-methyl-N-(2,4,6-trimethylphenyl)-5-thiazolecarboxamide;
4-Methyl-2-[[[(1-naphthalenylmethyl)amino]carbonyl]amino]-N-(2,4,6-trimethylphenyl)-5-thiazolecarboxamide
2-[[[Bis(phenylmethyl)amino]carbonyl]amino]-4-methyl-N-(2,4,6-trimethylphenyl)-5-thiazolecarboxamide;
2,6-Dimethyl-N-[4-methyl-5-[[(2,4,6-trimethylphenyl)amino]carbonyl]-2-thiazolyl]-4-morpholinecarboxamide;
2-Ethyl-N-[4-methyl-5-[[(2,4,6-trimethylphenyl)amino]carbonyl]-2-thiazolyl]-1-piperidinecarboxamide;
1-[[[4-Methyl-5-[[(2,4,6-trimethylphenyl)amino]carbonyl]-2-thiazolyl]amino]carbonyl]-3-piperidinecarboxylic acid ethyl ester;
3,3-Dimethyl-N-[4-methyl-5-[[(2,4,6-trimethylphenyl)amino]carbonyl]-2-thiazolyl]-1-piperidinecarboxamide
1-[[[4-Methyl-5-[[(2,4,6-trimethylphenyl)amino]carbonyl]-2-thiazolyl]amino]carbonyl]-4-piperidinecarboxylic acid ethyl ester;
4-Methyl-2-[[[(3-methyl-2-pyridinyl)amino]carbonyl]amino]-N-(2,4,6-trimethylphenyl)-5-thiazolecarboxamide
4-Methyl-2-[[[1-(phenylmethyl)-4-piperidinyl]amino]carbonyl]amino]-N-(2,4,6-trimethylphenyl)-5-thiazolecarboxamide;
Octahydro-N-[4-methyl-5-[[(2,4,6-trimethylphenyl)amino]carbonyl]-2-thiazolyl]-1 (2H)-quinolinecarboxamide;
3,4-Dihydro-N-[4-methyl-5-[[(2,4,6-trimethylphenyl)amino]carbonyl]-2-thiazolyl]-2(1H)-isoquinolinecarboxamide;
2-[[[(1,5-Dimethylhexyl)amino]carbonyl]amino]-4-methyl-N-(2,4,6-trimethylphenyl)-5-thiazolecarboxamide;
4-Methyl-2-[[[(1-methylheptyl)amino]carbonyl]amino]-N-(2,4,6-trimethylphenyl)-5-thiazolecarboxamide;
2-[[[[(2-Fluorophenyl)methyl]amino]carbonyl]amino]-4-methyl-N-(2,4,6-trimethylphenyl)-5-thiazolecarboxamide;
2-[[[[(2-Methoxyphenyl)methyl]amino]carbonyl]amino]-4-methyl-N-(2,4,6-trimethylphenyl)-5-thiazolecarboxamide;
2-[[[[(2-Ethoxyphenyl)methyl]amino]carbonyl]amino]-4-methyl-N-(2,4,6-trimethylphenyl)-5-thiazolecarboxamide;
2-[[[[(3-Methoxyphenyl)methyl]amino]carbonyl]amino]-4-methyl-N-(2,4,6-trimethylphenyl)-5-thiazolecarboxamide;
2-[[[[(4-Chlorophenyl)methyl]amino]carbonyl]amino]-4-methyl-N-(2,4,6-trimethylphenyl)-5-thiazolecarboxamide;
2-[[[[(4-Methoxyphenyl)methyl]amino]carbonyl]amino]-4-methyl-N-(2,4,6-trimethylphenyl)-5-thiazolecarboxamide;
2-[[[(2,2-Diphenylethyl)amino]carbonyl]amino]-4-methyl-N-(2,4,6-trimethylphenyl)-5-thiazolecarboxamide
2-[[[(2-Aminoethyl)phenylamino]carbonyl]amino]-4-methyl-N-(2,4,6-trimethylphenyl)-5-thiazolecarboxamide;
2-[[[[2-(3-Methoxyphenyl)ethyl]amino]carbonyl]amino]-4-methyl-N-(2,4,6-trimethylphenyl)-5-thiazolecarboxamide;
2-[[[[2-(3,4-Dimethoxyphenyl)ethyl]amino]carbonyl]amino]-4-methyl-N-(2,4,6-trimethylphenyl)-5-thiazolecarboxamide;
2-[[[[2-(4-Methoxyphenyl)ethyl]amino]carbonyl]amino]-4-methyl-N-(2,4,6-trimethylphenyl)-5-thiazolecarboxamide;
4-Methyl-2-[[[(3-phenylpropyl)amino]carbonyl]amino]-N-(2,4,6-trimethylphenyl)-5-thiazolecarboxamide;
2-[[[[2-(Cyclohex-1-en-1-yl)ethyl]amino]carbonyl]amino]-4-methyl-N-(2,4,6-trimethylphenyl)-5-thiazolecarboxamide;
2-[[[[4-(1,1-Dimethylethyl)cyclohexyl]amino]carbonyl]amino]-4-methyl-N-(2,4,6-trimethylphenyl)-5-thiazolecarboxamide;
2-[[[(3-Butoxypropyl)amino]carbonyl]amino]-4-methyl-N-(2,4,6-trimethylphenyl)-5-thiazolecarboxamide;
2-[[[[2-(2-Methoxyphenyl)ethyl]amino]carbonyl]amino]-4-methyl-N-(2,4,6-trimethylphenyl)-5-thiazolecarboxamide;
2-[[[[(2-Chloro-4-fluorophenyl)methyl]amino]carbonyl]amino]-4-methyl-N-(2,4,6-trimethylphenyl)-5-thiazolecarboxamide;
2-[[(Hexylmethylamino)carbonyl]amino]-4-methyl-N-(2,4,6-trimethylphenyl)-5-thiazolecarboxamide;
2-[[[[1-(4-Chlorophenyl)ethyl]amino]carbonyl]amino]-4-methyl-N-(2,4,6-trimethylphenyl)-5-thiazolecarboxamide;
2-[[[[2-(3-Chlorophenyl)ethyl]amino]carbonyl]amino]-4-methyl-N-(2,4,6-trimethylphenyl)-5-thiazolecarboxamide;
4-Methyl-2-[[[[2-(2-thienyl)ethyl]amino]carbonyl]amino]-N-(2,4,6-trimethylphenyl)-5-thiazolecarboxamide;
2-[[[[2-(2-Fluorophenyl)ethyl]amino]carbonyl]amino]-4-methyl-N-(2,4,6-trimethylphenyl)-5-thiazolecarboxamide;
4-Methyl-2-[[[[2-(2-pyridinyloxy)ethyl]amino]carbonyl]amino]-N-(2,4,6-trimethylphenyl)-5-thiazolecarboxamide;
2-[[[[(2-Bromo-4,5-dimethoxyphenyl)methyl]methylamino]carbonyl]amino]-4-methyl-N-(2,4,6-trimethylphenyl)-5-thiazolecarboxamide;
(E)-2-[[[(3,7-Dimethyl-2,6-octadienyl)amino]carbonyl]amino]-4-methyl-N-(2,4,6-trimethylphenyl)-5-thiazolecarboxamide;
2-[[[[(2,3-Dihydro-1,4-benzodioxin-2-yl)methyl]amino]carbonyl]amino]-4-methyl-N-(2,4,6-trimethylphenyl)-5-thiazolecarboxamide;
2-[[[[3-Methoxy-5-(trifluoromethyl)phenyl]amino]carbonyl]amino]-4-methyl-N-(2,4,6-trimethylphenyl)-5-thiazolecarboxamide;
2-[[[(4-Cyclohexylphenyl)amino]carbonyl]amino]-4-methyl-N-(2,4,6-trimethylphenyl)-5-thiazolecarboxamide;
4-Methyl-2-[[[(5,6,7,8-tetrahydro-1-naphthalenyl)amino]carbonyl]amino]-N-(2,4,6-trimethylphenyl)-5-thiazolecarboxamide;
2-[[(1-Anthracenylamino)carbonyl]amino]-4-methyl-N-(2,4,6-trimethylphenyl)-5-thiazolecarboxamide;
2-[[[(4-Chloro-1-naphthalenyl)amino]carbonyl]amino]-4-methyl-N-(2,4,6-trimethylphenyl)-5-thiazolecarboxamide;
4-Methyl-2-[[(2-naphthalenylamino)carbonyl]amino]-N-(2,4,6-trimethylphenyl)-5-thiazolecarboxamide;
2-[[(1H-Indol-5-ylamino)carbonyl]amino]-4-methyl-N-(2,4,6-trimethylphenyl)-5-thiazolecarboxamide;
2-[[(1,3-Benzodioxol-5-ylamino)carbonyl]amino]-4-methyl-N-(2,4,6-trimethylphenyl)-5-thiazolecarboxamide
4-Methyl-2-[[(2-pyrazinylamino)carbonyl]amino]-N-(2,4,6-trimethylphenyl)-5-thiazolecarboxamide;
2-[[[(5-Chloro-2-pyridinyl)amino]carbonyl]amino]-4-methyl-N-(2,4,6-trimethylphenyl)-5-thiazolecarboxamide;
4-Methyl-2-[[[(6-methyl-2-pyridinyl)amino]carbonyl]amino]-N-(2,4,6-trimethylphenyl)-5-thiazolecarboxamide;
4-Methyl-2-[[[(2-methyl-4-quinolinyl)amino]carbonyl]amino]-N-(2,4,6-trimethylphenyl)-5-thiazolecarboxamide;
2-[[[(2,3-Dihydro-1,4-benzodioxin-6-yl)amino]carbonyl]amino]-4-methyl-N-(2,4,6-trimethylphenyl)-5-thiazolecarboxamide;
2-[[([1,1′-Biphenyl]-2-ylamino)carbonyl]amino]-4-methyl-N-(2,4,6-trimethylphenyl)-5-thiazolecarboxamide;
2-[[[(4-Methoxy-2-methylphenyl)amino]carbonyl]amino]-4-methyl-N-(2,4,6-trimethylphenyl)-5-thiazolecarboxamide
4-Methyl-N-(2,4,6-trimethylphenyl)-2-[[[(2,4,6-trimethylphenyl)amino]carbonyl]amino]-5-thiazolecarboxamide;
2-[[[[2-(2-Hydroxyethyl)phenyl]amino]carbonyl]amino]-4-methyl-N-(2,4,6-trimethylphenyl)-5-thiazolecarboxamide;
2-[[[(3-Methoxyphenyl)amino]carbonyl]amino]-4-methyl-N-(2,4,6-trimethylphenyl)-5-thiazolecarboxamide;
2-[[[(4-Methoxy[1,1′-biphenyl]-3-yl)amino]carbonyl]amino]-4-methyl-N-(2,4,6-trimethylphenyl)-5-thiazolecarboxamide;
2-[[[(3-Acetylphenyl)amino]carbonyl]amino]-4-methyl-N-(2,4,6-trimethylphenyl)-5-thiazolecarboxamide;
2-[[[(4-Cyanophenyl)amino]carbonyl]amino]-4-methyl-N-(2,4,6-trimethylphenyl)-5-thiazolecarboxamide;
2-[[[[4-Fluoro-2-(trifluoromethyl)phenyl]amino]carbonyl]amino]-4-methyl-N-(2,4,6-trimethylphenyl)-5-thiazolecarboxamide
2-[[[(4-Hexyloxyphenyl)amino]carbonyl]amino]-4-methyl-N-(2,4,6-trimethylphenyl)-5-thiazolecarboxamide;
4-[[[[4-Methyl-5-[[(2,4,6-trimethylphenyl)amino]carbonyl]-2-thiazolyl]amino]carbonyl]amino]benzoic acid ethyl ester;
2-[[[(4-Decylphenyl)amino]carbonyl]amino]-4-methyl-N-(2,4,6-trimethylphenyl)-5-thiazolecarboxamide;
4-Methyl-2-[[[(4-propylphenyl)amino]carbonyl]amino]-N-(2,4,6-trimethylphenyl)-5-thiazolecarboxamide
4-Methyl-2-[[[(3,4,5-trimethoxyphenyl)amino]carbonyl]amino]-N-(2,4,6-trimethylphenyl)-5-thiazolecarboxamide;
4-Methyl-2-[[[[4-[[(5-methyl-3-isoxazolyl)amino]sulfonyl]phenyl]amino]carbonyl]amino]-N-(2,4,6-trimethylphenyl)-5-thiazolecarboxamide;
4-[[[[4-Methyl-5-[[(2,4,6-trimethylphenyl)amino]carbonyl]-2-thiazolyl]amino]carbonyl]amino]benzoic acid butyl ester;
2-[[(1-Isoquinolinylamino)carbonyl]amino]-4-methyl-N-(2,4,6-trimethylphenyl)-5-thiazolecarboxamide;
4-Methyl-2-[[[[2-[(phenylmethyl)thio]phenyl]amino]carbonyl]amino]-N-(2,4,6-trimethylphenyl)-5-thiazolecarboxamide;
4-Methyl-2-[[[[4-[(5-phenoxypentyl)oxy]phenyl]amino]carbonyl]amino]-N-(2,4,6-trimethylphenyl)-5-thiazolecarboxamide;
2-[[[[5-(1,1-Dimethylpropyl)-2-methoxyphenyl]amino]carbonyl]amino]-4-methyl-N-(2,4,6-trimethylphenyl)-5-thiazolecarboxamide;
2-[[[(1,2-Dihydro-5-acenaphthylenyl)amino]carbonyl]amino]-4-methyl-N-(2,4,6-trimethylphenyl)-5-thiazolecarboxamide;
4-Methyl-2-[[[(3-phenoxyphenyl)amino]carbonyl]amino]-N-(2,4,6-trimethylphenyl)-5-thiazolecarboxamide
4-Methyl-2-[[[[2-(4-morpholinyl)phenyl]amino]carbonyl]amino]-N-(2,4,6-trimethylphenyl)-5-thiazolecarboxamide;
4-Methyl-2-[[[[2-(1-piperidinyl)phenyl]amino]carbonyl]amino]-N-(2,4,6-trimethylphenyl)-5-thiazolecarboxamide;
2-[[[(1-Acetyl-2,3-dihydro-1H-indol-6-yl)amino]carbonyl]amino]-4-methyl-N-(2,4,6-trimethylphenyl)-5-thiazolecarboxamide;
2-[[[(2-Bromo-5-methoxyphenyl)amino]carbonyl]amino]-4-methyl-N-(2,4,6-trimethylphenyl)-5-thiazolecarboxamide;
2-[[[(2,3-Dimethyl-1H-indol-5-yl)amino]carbonyl]amino]-4-methyl-N-(2,4,6-trimethylphenyl)-5-thiazolecarboxamide;
4-Methyl-2-[[[[2-[[(1-methylethyl)amino]carbonyl]phenyl]amino]carbonyl]amino]-N-(2,4,6-trimethylphenyl)-5-thiazolecarboxamide;
2-[[[(3-Bromo-2-methylphenyl)amino]carbonyl]amino]-4-methyl-N-(2,4,6-trimethylphenyl)-5-thiazolecarboxamide;
2-[[[(4-Methoxybutyl)amino]carbonyl]amino]-4-methyl-N-(2,4,6-trimethylphenyl)-5-thiazolecarboxamide
2-[[[(3,3-Dimethylbutyl)amino]carbonyl]amino]-4-methyl-N-(2,4,6-trimethylphenyl)-5-thiazolecarboxamide;
4-Methyl-2-[[[(2-methylbutyl)amino]carbonyl]amino]-N-(2,4,6-trimethylphenyl)-5-thiazolecarboxamide;
4-Methyl-2-[[[(3-methylbutyl)amino]carbonyl]amino]-N-(2,4,6-trimethylphenyl)-5-thiazolecarboxamide;
2-[[[(2-Methoxyethyl)amino]carbonyl]amino]-4-methyl-N-(2,4,6-trimethylphenyl)-5-thiazolecarboxamide;
2-[[[[2-(Dimethylamino)ethyl]amino]carbonyl]amino]-4-methyl-N-(2,4,6-trimethylphenyl)-5-thiazolecarboxamide;
4-Methyl-2-[[[[2-(methylthio)ethyl]amino]carbonyl]amino]-N-(2,4,6-trimethylphenyl)-5-thiazolecarboxamide;
2-[[(Butylamino)carbonyl]amino]-N-(2,3-dihydro-1H-inden-5-yl)-4-methyl-5-thiazolecarboxamide;
2-[[(Butylamino)carbonyl]amino]-N-2-naphthalenyl-4-methyl-5-thiazolecarboxamide;
2-[[(Butylamino)carbonyl]amino]-N-(3-hydroxy-2-naphthalenyl)-4-methyl-5-thiazolecarboxamide;
2-[[(Butylamino)carbonyl]amino]-N-(2-fluoro-5-methylphenyl)-4-methyl-5-thiazolecarboxamide;
2-[[(Butylamino)carbonyl]amino]-N-(2,6-dimethylphenyl)-4-methyl-5-thiazolecarboxamide;
N-(3-Bromo-2,4,6-trimethylphenyl)-2-[[(butylamino)carbonyl]amino]-4-methyl-5-thiazolecarboxamide;
2-[[(Butylamino)carbonyl]amino]-N-[2,6-dimethyl-3-(1-methylethyl)phenyl]-4-methyl-5-thiazolecarboxamide
N-(2-Bromo-4,6-dimethylphenyl)-2-[[(butylamino)carbonyl]amino]-4-methyl-5-thiazolecarboxamide;
3-[[[2-[[(Butylamino)carbonyl]amino]-4-methyl-5-thiazolyl]carbonyl]amino]-4-methyl-2-thiophenecarboxylic acid methyl ester;
2-[[(Butylamino)carbonyl]amino]-4-methyl-N-(2-methyl-6-quinolinyl)-5-thiazolecarboxamide;
2-[[(Butylamino)carbonyl]amino]-N-(2,6-dimethoxyphenyl)-4-methyl-5-thiazolecarboxamide;
2-[[(Butylamino)carbonyl]amino]-N-(4-methoxy-2-naphthalenyl)-4-methyl-5-thiazolecarboxamide;
2-[[(Butylamino)carbonyl]amino]-N-(2-methyl-1-naphthalenyl)-4-methyl-5-thiazolecarboxamide;
2-[[(Butylamino)carbonyl]amino]-N-[4-(dimethylamino)-2,3,5,6-tetramethylphenyl]-4-methyl-5-thiazolecarboxamide;
2-[[(Butylamino)carbonyl]amino]-N-(6-methyl-5-quinolinyl)-4-methyl-5-thiazolecarboxamide;
2-[[(Butylamino)carbonyl]amino]-N-[2-(2-hydroxyethyl)-6-methylphenyl]-4-methyl-5-thiazolecarboxamide;
2-[[(Butylamino)carbonyl]amino]-N-(2,6-dimethyl-3-nitrophenyl)-4-methyl-5-thiazolecarboxamide;
N-(2-Bromo-3,4,6-trimethylphenyl)-2-[[(butylamino)carbonyl]amino]-4-methyl-5-thiazolecarboxamide;
N-(2-Acetyl-6-hydroxyphenyl)-2-[[(butylamino)carbonyl]amino]-4-methyl-5-thiazolecarboxamide;
[4-[[[2-[[(Butylamino)carbonyl]amino]-4-methyl-5-thiazolyl]carbonyl]amino]-2,3,5,6-tetramethylphenyl]carbamic acid 1,1-dimethylethyl ester;
2-[[(Butylamino)carbonyl]amino]-N-(2,6-dichlorophenyl)-4-methyl-5-thiazolecarboxamide;
N-(4-Amino-2,3,5,6-tetramethylphenyl)-2-[[(butylamino)carbonyl]amino]-4-methyl-5-thiazolecarboxamide;
N-[5-(Acetylamino)-2,4-dimethylphenyl]-2-[[(butylamino)carbonyl]amino]-4-methyl-5-thiazolecarboxamide;
N-(4-Bromo-2,6-dimethylphenyl)-2-[[(butylamino)carbonyl]amino]-4-methyl-5-thiazolecarboxamide
2-[[(Butylamino)carbonyl]amino]-N-(2-chloro-6-methylphenyl)-4-methyl-5-thiazolecarboxamide;
4-Methyl-2-[(methylsulfonyl)amino]-N-(2,4,6-trimethylphenyl)-5-thiazolecarboxamide;
4-Methyl-2-[[(phenylamino)thiocarbonyl]amino]-N-(2,4,6-trimethylphenyl)-5-thiazolecarboxamide;
2-[[(Ethylamino)carbonyl]amino]-4-methyl-N-(2,4,6-trimethylphenyl)-5-thiazolecarboxamide;
N-(2-Chloro-6-methylphenyl)-2-[(cyclopropylcarbonyl)amino]-5-thiazolecarboxamide;
2-[[[(1,1-Dimethylethyl)amino]carbonyl]amino]-N-(2-chloro-6-methylphenyl)-5-thiazolecarboxamide;
2-[[(1,1-Dimethylethoxy)carbonyl]amino]-4-methyl-N-(2,4,6-trimethylphenyl)-5-thiazoleacetamide;
2-Amino-4-methyl-N-(2,4,6-trimethylphenyl)-5-thiazoleacetamide;
N-(2-Chloro-6-methylphenyl)-2-[(4,6-dimethyl-2-pyridinyl)amino]-5-thiazolecarboxamide;
N-(2-Chloro-6-methylphenyl)-2-[(4-ethyl-2-pyridinyl)amino]-5-thiazolecarboxamide;
N-(2-Chloro-6-methylphenyl)-2-[(2,6-dimethyl-4-pyrimidinyl)amino]-5-thiazolecarboxamide;
N-(2-Chloro-6-methylphenyl)-2-(3-pyridazinylamino)-5-thiazolecarboxamide;
N-(2-Chloro-6-methylphenyl)-4-methyl-2-[(2-thienylcarbonyl)amino]-5-thiazolecarboxamide;
N-(2-Chloro-6-methylphenyl)-2-[(cyclopropylcarbonyl)amino]-4-methyl-5-thiazolecarboxamide;
N-(2-Chloro-6-methylphenyl)-4-methyl-2-[(2-furanylcarbonyl)amino]-5-thiazolecarboxamide;
N-(2-Chloro-6-methylphenyl)-4-methyl-2-[(3-thienylcarbonyl)amino]-5-thiazolecarboxamide;
N-(2-Chloro-6-methylphenyl)-4-methyl-2-[(3-furanylcarbonyl)amino]-5-thiazolecarboxamide;
trans-N-(2-Chloro-6-methylphenyl)-4-methyl-2-[[(2-phenylcyclopropyl)carbonyl]amino]-5-thiazolecarboxamide;
N-(2-Chloro-6-methylphenyl)-4-methyl-2-[[(2-methylcyclopropyl)carbonyl]amino]-5-thiazolecarboxamide;
N-(2-Chloro-6-methylphenyl)-2-[(cyclobutylcarbonyl)amino]-4-methyl-5-thiazolecarboxamide;
N-(2-Chloro-6-methylphenyl)-2-[(cyclopentylcarbonyl)amino]-4-methyl-5-thiazolecarboxamide;
2-(Benzoylamino)-N-(2-chloro-6-methylphenyl)-4-methyl-5-thiazolecarboxamide;
N-(2-Chloro-6-methylphenyl)-2-[(1-oxopropyl)amino]-5-thiazolecarboxamide;
N-(2-Chloro-6-methylphenyl)-2-[(1-oxobutyl)amino]-5-thiazolecarboxamide;
N-(2-Chloro-6-methylphenyl)-2-[(2-ethyl-1-oxobutyl)amino]-5-thiazolecarboxamide;
N-(2-Chloro-6-methylphenyl)-2-[[(1-phenylcyclopropyl)carbonyl]amino]-5-thiazolecarboxamide;
N-(2-Chloro-6-methylphenyl)-2-[[(1-methylcyclopropyl)carbonyl]amino]-5-thiazolecarboxamide;
N-(2-Chloro-6-methylphenyl)-2-[[(2,2-dichloro-1-methylcyclopropyl)carbonyl]amino]-5-thiazolecarboxamide;
N-(2-Chloro-6-methylphenyl)-2-[[(2-methylcyclopropyl)carbonyl]amino]-5-thiazolecarboxamide;
N-(2-Chloro-6-methylphenyl)-2-[[(1-hydroxycyclopropyl)carbonyl]amino]-5-thiazolecarboxamide;
N-(2-Chloro-6-methylphenyl)-2-[[(2,2,3,3-tetramethylcyclopropyl)carbonyl]amino]-5-thiazolecarboxamide;
N-(2-Chloro-6-methylphenyl)-2-[[(1-cyanocyclopropyl)carbonyl]amino]-5-thiazolecarboxamide;
N-(2-Chloro-6-methylphenyl)-2-[(cyclobutylcarbonyl)amino]-5-thiazolecarboxamide;
N-(2-Chloro-6-methylphenyl)-2-[(cyclopentylcarbonyl)amino]-5-thiazolecarboxamide;
N-(2-Chloro-6-methylphenyl)-2-[(cyclohexylcarbonyl)amino]-5-thiazolecarboxamide;
N-(2-Chloro-6-methylphenyl)-2-[(phenylacetyl)amino]-5-thiazolecarboxamide;
N-(2-Chloro-6-methylphenyl)-2-[(cyclohexylacetyl)amino]-5-thiazolecarboxamide;
N-(2-Chloro-6-methylphenyl)-2-[(4-pyridinylacetyl)amino]-5-thiazolecarboxamide;
N-(2-Chloro-6-methylphenyl)-2-[[(2,5-dimethyl-1H-pyrrol-3-yl)carbonyl]amino]-5-thiazolecarboxamide;
N-(2-Chloro-6-methylphenyl)-2-[(2-pyridinylcarbonyl)amino]-5-thiazolecarboxamide;
N-(2-Chloro-6-methylphenyl)-2-[(3-pyridinylcarbonyl)amino]-5-thiazolecarboxamide;
N-(2-Chloro-6-methylphenyl)-2-[(4-pyridinylcarbonyl)amino]-5-thiazolecarboxamide;
N-(2-Chloro-6-methylphenyl)-2-[(3-thienylcarbonyl)amino]-5-thiazolecarboxamide;
N-(2-Chloro-6-methylphenyl)-2-[(2-thienylcarbonyl)amino]-5-thiazolecarboxamide;
N-(2-Chloro-6-methylphenyl)-2-[(2-furanylcarbonyl)amino]-5-thiazolecarboxamide;
N-(2-Chloro-6-methylphenyl)-2-[(3-furanylcarbonyl)amino]-5-thiazolecarboxamide;
trans-N-(2-Chloro-6-methylphenyl)-2-[[(2-phenylcyclopropyl)carbonyl]amino]-5-thiazolecarboxamide;
N-(2-Chloro-6-methylphenyl)-2-[(2-methyl-1-oxopentyl)amino]-5-thiazolecarboxamide;
2-(Benzoylamino)-N-(2-chloro-6-methylphenyl)-5-thiazolecarboxamide;
2-[(Cyclopropylcarbonyl)amino]-N-(2,6-dimethylphenyl)-5-thiazolecarboxamide;
2-[(Cyclopropylcarbonyl)amino]-N-(2,4,6-trimethylphenyl)-5-thiazolecarboxamide;
N-(2-Chloro-4,6-dimethylphenyl)-2-[(cyclopropylcarbonyl)amino]-5-thiazolecarboxamide;
[4-[2-Oxo-2-[(2,4,6-trimethylphenyl)amino]ethyl]-2-thiazolyl]carbamic acid 1,1-dimethylethyl ester;
2-Amino-N-(2,4,6-trimethylphenyl)-4-thiazoleacetamide;
5-Amino-2-methyl-N-(2,4,6-trimethylphenyl)benzamide;
2-Amino-5-chloro-N-(2,4,6-trimethylphenyl)-4-pyrimidinecarboxamide;
[4-Methyl-5-[[(2,4,6-trimethylphenyl)amino]carbonyl]-2-oxazolyl]carbamic acid 1,1-dimethylethyl ester;
2-Amino-4-(methyl)-N-(2,4,6-trimethylphenyl)-5-oxazolecarboxamide, trifluoroacetate (1:1);
2-Amino-N-(2,4,6-trimethylphenyl)-5-pyridinecarboxamide;
3-Amino-N-(2,4,6-trimethylphenyl)-4-pyridinecarboxamide;
N-(2-Chloro-6-methylphenyl)-2-[(4-methyl-2-pyridinyl)amino]-5-thiazolecarboxamide;
2-[(6-Amino-2-pyridinyl)amino]-N-(2-chloro-6-methylphenyl)-5-thiazolecarboxamide;
N-(2-Chloro-6-methylphenyl)-2-[(6-propyl-2-pyridinyl)amino]-5-thiazolecarboxamide;
N-(2-Chloro-6-methylphenyl)-2-[(6-ethyl-4-pyrimidinyl)amino]-5-thiazolecarboxamide;
′N-(2-Chloro-6-methylphenyl)-2-(2-pyridinyl amino)-5-thiazolecarboxamide;
′N-(2-Chloro-6-methylphenyl)-2-[(6-methyl-2-pyridinyl)amino]-5-thiazolecarboxamide;
′N-(2-Chloro-6-methylphenyl)-2-[(5-methyl-2-pyridinyl)amino]-5-thiazolecarboxamide;
′N-(2-Chloro-6-methylphenyl)-2-[(4-methyl-2-pyridinyl)amino]-5-thiazolecarboxamide;
′N-(2-Chloro-6-methylphenyl)-2-[(3-methyl-2-pyridinyl)amino]-5-thiazolecarboxamide;
′2-[(5-Bromo-3-methyl-2-pyridinyl)amino]-N-(2-chloro-6-methylphenyl)-5-thiazolecarboxamide;
′2-[(6-Amino-2-pyridinyl)amino]-N-(2-chloro-6-methylphenyl)-5-thiazolecarboxamide;
′2-[(5-Bromo-2-pyridinyl)amino]-N-(2-chloro-6-methylphenyl)-5-thiazolecarboxamide;
′N-(2-Chloro-6-methylphenyl)-2-[[3-(phenylmethoxy)-2-pyridinyl]amino]-5-thiazolecarboxamide;
′N-(2-Chloro-6-methylphenyl)-2-[(5-chloro-2-pyridinyl)amino]-5-thiazolecarboxamide;
′N-(2-Chloro-6-methylphenyl)-2-[(6-ethyl-2-pyridinyl)amino]-5-thiazolecarboxamide;
′N-(2-Chloro-6-methylphenyl)-2-[(6-propyl-2-pyridinyl)amino]-5-thiazolecarboxamide;
′2-[(3-Bromo-5-methyl-2-pyridinyl)amino]-N-(2-chloro-6-methylphenyl)-5-thiazolecarboxamide;
′2-[(2-Amino-3-pyridinyl)amino]-N-(2-chloro-6-methylphenyl)-5-thiazolecarboxamide;
′2-[(3-Amino-2-pyridinyl)amino]-N-(2-chloro-6-methylphenyl)-5-thiazolecarboxamide;
′N-(2-Chloro-6-methylphenyl)-2-(4-pyridinylamino)-5-thiazolecarboxamide;
′N-(2-Chloro-6-methylphenyl)-2-(3-pyridinylamino)-5-thiazolecarboxamide;
′N-(2-Chloro-6-methylphenyl)-2-[(6-chloro-3-pyridinyl)amino]-5-thiazolecarboxamide;
′N-(2-Chloro-6-methylphenyl)-2-[(2-chloro-3-pyridinyl)amino]-5-thiazolecarboxamide;
′N-(2-Chloro-6-methylphenyl)-2-[(6-methoxy-3-pyridinyl)amino]-5-thiazolecarboxamide;
′N-(2-Chloro-6-methylphenyl)-2-[(3,5-dimethyl-2-pyrazinyl)amino]-5-thiazolecarboxamide;
′N-(2-Chloro-6-methylphenyl)-2-(phenylamino)-5-thiazolecarboxamide;
′N-(2-Chloro-6-methylphenyl)-2-[(3-ethylphenyl)amino]-5-thiazolecarboxamide;
′N-(2-Chloro-6-methylphenyl)-2-[(3,5-dimethylphenyl)amino]-5-thiazolecarboxamide;
′N-(2-Chloro-6-methylphenyl)-2-[(4,6-dimethyl-2-pyrimidinyl)amino]-5-thiazolecarboxamide;
′N-(2-Chloro-6-methylphenyl)-2-[(6-ethyl-4-pyrimidinyl)amino]-5-thiazolecarboxamide;
′N-(2-Chloro-6-methylphenyl)-2-[(6-chloro-2-pyrazinyl)amino]-5-thiazolecarboxamide;
′2-[(3-Aminophenyl)amino]-N-(2-chloro-6-methylphenyl)-5-thiazolecarboxamide;
′N-(2-Chloro-6-methylphenyl)-2-[(3-hydroxyphenyl)amino]-5-thiazolecarboxamide;
′2-[(3-Bromophenyl)amino]-N-(2-chloro-6-methylphenyl)-5-thiazolecarboxamide;
′N-(2,6-Dimethylphenyl)-2-(phenylamino)-5-thiazolecarboxamide;
′N-(2,6-Dimethylphenyl)-2-(methylphenyl amino)-5-thiazolecarboxamide;
′N-(2,6-Dimethylphenyl)-2-(2-pyridinylamino)-5-thiazolecarboxamide;
′N-(2,6-Dimethylphenyl)-2-[(6-methyl-2-pyridinyl)amino]-5-thiazolecarboxamide;
′N-(2,6-Dimethylphenyl)-2-[(4-methyl-2-pyridinyl)amino]-5-thiazolecarboxamide;
′N-(2,6-Dimethylphenyl)-2-[(4-ethyl-2-pyridinyl)amino]-5-thiazolecarboxamide;
′N-(2,6-Dimethylphenyl)-2-[(4,6-dimethyl-2-pyridinyl)amino]-5-thiazolecarboxamide;
′2-[(6-Amino-2-pyridinyl)amino]-N-(2,6-dimethylphenyl)-5-thiazolecarboxamide;
′N-(2,6-Dimethylphenyl)-2-[(6-ethyl-2-pyridinyl)amino]-5-thiazolecarboxamide;
′N-(2,6-Dimethylphenyl)-2-[(6-propyl-2-pyridinyl)amino]-5-thiazolecarboxamide;
′2-[(2-Amino-3-pyridinyl)amino]-N-(2,6-dimethylphenyl)-5-thiazolecarboxamide;
′2-[(3-Amino-2-pyridinyl)amino]-N-(2,6-dimethylphenyl)-5-thiazolecarboxamide;
′2-[(6-Amino-2-methyl-4-pyrimidinyl)amino]-N-(2,6-dimethylphenyl)-5-thiazolecarboxamide;
′N-(2,6-Dimethylphenyl)-2-[[6-(4-morpholinyl)-3-pyridazinyl]amino]-5-thiazolecarboxamide;
′2-[(6-Chloro-3-pyridazinyl)amino]-N-(2,6-dimethylphenyl)-5-thiazolecarboxamide;
′N-(2,6-Dimethylphenyl)-2-(3-pyridazinylamino)-5-thiazolecarboxamide;
′2-[(3-Aminophenyl)amino]-N-(2,6-dimethylphenyl)-5-thiazolecarboxamide;
′2-[(3-Bromophenyl)amino]-N-(2,6-dimethylphenyl)-5-thiazolecarboxamide;
′2-(2-Pyridinylamino)-N-(2,4,6-tri methylphenyl)-5-thiazolecarboxamide;
′2-[(6-Methyl-2-pyridinyl)amino]-N-(2,4,6-trimethylphenyl)-5-thiazolecarboxamide;
′2-[(5-Methyl-2-pyridinyl)amino]-N-(2,4,6-trimethylphenyl)-5-thiazolecarboxamide;
′2-[(4-Methyl-2-pyridinyl)amino]-N-(2,4,6-trimethylphenyl)-5-thiazolecarboxamide;
′2-[(3-Methyl-2-pyridinyl)amino]-N-(2,4,6-trimethylphenyl)-5-thiazolecarboxamide;
′2-[(5-Bromo-2-pyridinyl)amino]-N-(2,4,6-trimethylphenyl)-5-thiazolecarboxamide;
′2-[(5-Chloro-2-pyridinyl)amino]-N-(2,4,6-trimethylphenyl)-5-thiazolecarboxamide;
′2-[(6-Methoxy-3-pyridinyl)amino]-N-(2,4,6-trimethylphenyl)-5-thiazolecarboxamide;
′2-[(4-Ethyl-2-pyridinyl)amino]-N-(2,4,6-trimethylphenyl)-5-thiazolecarboxamide;
′2-[(6-Ethyl-2-pyridinyl)amino]-N-(2,4,6-trimethylphenyl)-5-thiazolecarboxamide;
′2-[(6-Chloro-3-pyridinyl)amino]-N-(2,4,6-trimethylphenyl)-5-thiazolecarboxamide;
′2-[(2,6-Dimethyl-4-pyrimidinyl)amino]-N-(2,4,6-trimethylphenyl)-5-thiazolecarboxamide;
′2-[(4-Methyl-2-pyrimidinyl)amino]-N-(2,4,6-trimethylphenyl)-5-thiazolecarboxamide;
′2-(2-Pyrazinylamino)-N-(2,4,6-trimethylphenyl)-5-thiazolecarboxamide
′2-[(6-Chloro-2-pyrazinyl)amino]-N-(2,4,6-trimethylphenyl)-5-thiazolecarboxamide;
′2-[(3,5-Dimethyl-2-pyrazinyl)amino]-N-(2,4,6-trimethylphenyl)-5-thiazolecarboxamide;
′N-(2-Chloro-6-methylphenyl)-2-[[2-methyl-6-[[2-(4-morpholinyl)ethyl]amino]-4-pyrimidinyl]amino]-5-thiazolecarboxamide;
′N-(2-Chloro-6-methylphenyl)-2-[[2-methyl-6-[[3-(4-morpholinyl)propyl]amino]-4-pyrimidinyl]amino]-5-thiazolecarboxamide;
′N-(2-Chloro-6-methylphenyl)-2-[[2-methyl-6-[methyl[3-(methylamino)propyl]amino]-4-pyrimidinyl]amino]-5-thiazolecarboxamide;
′N-(2-Chloro-6-methylphenyl)-2-[[2-methyl-6-[[2-(tetrahydro-2-oxo-1H-imidazol-1-yl)ethyl]amino]-4-pyrimidinyl]amino]-5-thiazolecarboxamide;
′N-(2-Chloro-6-methylphenyl)-2-[[2-methyl-6-[(2-1H-imidazol-4-ylethyl)amino]-4-pyrimidinyl]amino]-5-thiazolecarboxamide;
′N-(2-Chloro-6-methylphenyl)-2-[[2-methyl-6-(4-morpholinyl)-4-pyrimidinyl]amino]-5-thiazolecarboxamide;
′N-(2-Chloro-6-methylphenyl)-2-[[6-[[[(2R)-1-ethyl-2-pyrrolidinyl]methyl]amino]-2-methyl-4-pyrimidinyl]amino]-5-thiazolecarboxamide;
′N-(2-Chloro-6-methylphenyl)-2-[[6-[[[(2S)-1-ethyl-2-pyrrolidinyl]methyl]amino]-2-methyl-4-pyrimidinyl]amino]-5-thiazolecarboxamide;
′2-[[6-[(2S)-2-(Aminocarbonyl)-1-pyrrolidinyl]-2-methyl-4-pyrimidinyl]amino]-N-(2-chloro-6-methylphenyl)-5-thiazolecarboxamide;
′N-(2-Chloro-6-methylphenyl)-2-[[6-[(2-hydroxyethyl)amino]-2-methyl-4-pyrimidinyl]amino]-5-thiazolecarboxamide;
′N-(2-Chloro-6-methylphenyl)-2-[[6-[4-(hydroxymethyl)-1-piperidinyl]-2-methyl-4-pyrimidinyl]amino]-5-thiazolecarboxamide;
′N-(2-Chloro-6-methylphenyl)-2-[[6-[4-(2-hydroxyethyl)-1-piperazinyl]-2-methyl-4-pyrimidinyl]amino]-5-thiazolecarboxamide;
′1-[6-[[5-[[(2-Chloro-6-methylphenyl)amino]carbonyl]-2-thiazolyl]amino]-2-methyl-4-pyrimidinyl]-4-piperidinecarboxamide;
′N-(2-Chloro-6-methylphenyl)-2-[[2-methyl-6-[(3S)-3-methyl-1-piperazinyl]-4-pyrimidinyl]amino]-5-thiazolecarboxamide;
′2-[[6-[3-(Acetylamino)-1-pyrrolidinyl]-2-methyl-4-pyrimidinyl]amino]-N-(2-chloro-6-methylphenyl)-5-thiazolecarboxamide;
′N-(2-Chloro-6-methylphenyl)-2-[[6-[[2-(1-methyl-2-pyrrolidinyl)ethyl]amino]-2-methyl-4-pyrimidinyl]amino]-5-thiazolecarboxamide;
′N-(2-Chloro-6-methylphenyl)-2-[[2-methyl-6-[[(5-methyl-2-pyrazinyl)methyl]amino]-4-pyrimidinyl]amino]-5-thiazolecarboxamide;
′N-(2-Chloro-6-methylphenyl)-2-[[2-methyl-6-[[2-(1H-1,2,3-triazol-1-yl)ethyl]amino]-4-pyrimidinyl]amino]-5-thiazolecarboxamide;
′N-(2-Chloro-6-methylphenyl)-2-[[6-[[2-(4-morpholinyl)ethyl]amino]-4-pyrimidinyl]amino]-5-thiazolecarboxamide;
′N-(2-Chloro-6-methylphenyl)-2-[[6-[[2-(dimethylamino)ethyl]amino]-4-pyrimidinyl]amino]-5-thiazolecarboxamide;
′N-(2-Chloro-6-methylphenyl)-2-[[6-[[2-(tetrahydro-2-oxo-1H-imidazol-1-yl)ethyl]amino]-4-pyrimidinyl]amino]-5-thiazolecarboxamide;
′N-(2-Chloro-6-methylphenyl)-2-[[6-[methyl[2-(methylamino)ethyl]amino]-4-pyrimidinyl]amino]-5-thiazolecarboxamide;
′N-(2-Chloro-6-methylphenyl)-2-[[6-[[2-(1-methyl-2-pyrrolidinyl)ethyl]amino]-4-pyrimidinyl]amino]-5-thiazolecarboxamide;
′N-(2-Chloro-6-methylphenyl)-2-[[6-[[2-(1-pyrrolidinyl)ethyl]amino]-4-pyrimidinyl]amino]-5-thiazolecarboxamide;
′N-(2-Chloro-6-methylphenyl)-2-[[6-[[(1-ethyl-2-pyrrolidinyl)methyl]amino]-4-pyrimidinyl]amino]-5-thiazolecarboxamide;
′N-(2-Chloro-6-methylphenyl)-2-[[6-[(4-piperidinylmethyl)amino]-4-pyrimidinyl]amino]-5-thiazolecarboxamide;
′2-[[6-[[2-(Acetylamino)ethyl]amino]-4-pyrimidinyl]amino]-N-(2-chloro-6-methylphenyl)-5-thiazolecarboxamide;
′N-(2-Chloro-6-methylphenyl)-2-[[6-[[2-(1H-1,2,3-triazol-1-yl)ethyl]amino]-4-pyrimidinyl]amino]-5-thiazolecarboxamide;
′N-(2-Chloro-6-methylphenyl)-2-[[6-(4-morpholinyl)-4-pyrimidinyl]amino]-5-thiazolecarboxamide;
′N-(2-Chloro-6-methylphenyl)-2-[[6-[[2-(4-morpholinyl)ethyl]amino]-2-pyridinyl]amino]-5-thiazolecarboxamide;
′N-(2-Chloro-6-methylphenyl)-2-[[6-[[3-(4-morpholinyl)propyl]amino]-2-pyridinyl]amino]-5-thiazolecarboxamide;
′N-(2-Chloro-6-methylphenyl)-2-[[6-[methyl[3-(methylamino)propyl]amino]-2-pyridinyl]amino]-5-thiazolecarboxamide;
′N-(2-Chloro-6-methylphenyl)-2-[[6-[(3S)-3-methyl-1-piperazinyl]-2-pyridinyl]amino]-5-thiazolecarboxamide;
′N-(2-Chloro-6-methylphenyl)-2-[[6-[(3-1H-imidazol-1-ylpropyl)amino]-2-pyridinyl]amino]-5-thiazolecarboxamide;
′N-(2-Chloro-6-methylphenyl)-2-[[6-[(2-hydroxyethyl)amino]-2-pyridinyl]amino]-5-thiazolecarboxamide;
′N-(2-Chloro-6-methylphenyl)-2-[[6-[(2-1H-imidazol-1-ylethyl)amino]-2-pyridinyl]amino]-5-thiazolecarboxamide;
′N-(2-Chloro-6-methylphenyl)-2-[[6-(4-morpholinyl)-2-pyridinyl]amino]-5-thiazolecarboxamide;
′N-(2-Chloro-6-methylphenyl)-2-[[6-[[2-(4-morpholinyl)ethyl]amino]-2-pyrazinyl]amino]-5-thiazolecarboxamide;
′N-(2-Chloro-6-methylphenyl)-2-[[6-[[3-(4-morpholinyl)propyl]amino]-2-pyrazinyl]amino]-5-thiazolecarboxamide;
′N-(2-Chloro-6-methylphenyl)-2-[[6-(4-morpholinyl)-2-pyrazinyl]amino]-5-thiazolecarboxamide;
′N-(2-Chloro-6-methylphenyl)-2-[[6-[(3S)-3-methyl-1-piperazinyl]-2-pyrazinyl]amino]-5-thiazolecarboxamide;
′N-(2-Chloro-6-methylphenyl)-2-[[6-(3-hydroxy-1-pyrrolidinyl)-2-pyrazinyl]amino]-5-thiazolecarboxamide;
′N-(2-Chloro-6-methylphenyl)-2-[[6-(1H-imidazol-1-yl)-2-pyrazinyl]amino]-5-thiazolecarboxamide;
′N-(2-Chloro-6-methylphenyl)-2-[[6-(3-hydroxy-1-pyrrolidinyl)-3-pryidazinyl]amino]-5-thiazolecarboxamide;
′N-(2-Chloro-6-methylphenyl)-2-[[6-(1H-imidazol-1-yl)-3-pyridazinyl]amino]-5-thiazolecarboxamide;
′N-(2-Chloro-6-methylphenyl)-2-[[3-(methylamino)-2-pyrazinyl]amino]-5-thiazolecarboxamide;
′N-(2-Chloro-6-methylphenyl)-2-[[3-(3-hydroxy-1-pyrrolidinyl)-2-pyrazinyl]amino]-5-thiazolecarboxamide;
′N-(2-Chloro-6-methylphenyl)-2-[[3-(cyclopropylamino)-2-pyrazinyl]amino]-5-thiazolecarboxamide;
′N-(2-Chloro-6-methylphenyl)-2-[[3-(4-morpholinyl)-2-pyrazinyl]amino]-5-thiazolecarboxamide;
′N-(2-Chloro-6-methylphenyl)-2-[[3-[[2-(4-morpholinyl)ethyl]amino]-2-pyrazinyl]amino]-5-thiazolecarboxamide;
′2-[[3-[[2-(Acetylamino)ethyl]amino]-2-pyrazinyl]amino]-N-(2-chloro-6-methylphenyl)-5-thiazolecarboxamide;
′N-(2-Chloro-6-methylphenyl)-2-(cyclohexylamino)-5-thiazolecarboxamide;
′N-(2-Chloro-6-methylphenyl)-2-(methylamino)-5-thiazolecarboxamide;
′N-(2-Chloro-6-methylphenyl)-2-(cyclopropylamino)-5-thiazolecarboxamide;
′N-(2-Chloro-6-methylphenyl)-2-[(phenylmethyl)amino]-5-thiazolecarboxamide;
′2-[[2-(Acetylamino)ethyl]amino]-N-(2-chloro-6-methylphenyl)-5-thiazolecarboxamide;
′N-(2-Chloro-6-methylphenyl)-2-[[(1R)-1-(hydroxymethyl)-3-methylbutyl]amino]-5-thiazolecarboxamide;
′N-(2-Chloro-6-methylphenyl)-2-[[6-(methoxymethyl)-4-pyrimidinyl]amino]-5-thiazolecarboxamide;
′N-(2-Chloro-6-methylphenyl)-2-[[6-hydroxymethyl)-4-pyrimidinyl]amino]-5-thiazolecarboxamide;
′N-(2-Chloro-6-methylphenyl)-2-[[6-(4-morpholinylmethyl)-4-pyrinmidinyl]amino]-5-thiazolecarboxamide;
′N-(2-Chloro-6-methylphenyl)-2-[[6-[[[2-(dimethylamino)-ethyl]amino]methyl]-4-pyrimidinyl]amino]-5-thiazolecarboxamide;
′N-(2-Chloro-6-methylphenyl)-2-[[6-[[[2-(4-morpholinyl)ethyl]amino]methyl]-4-pyrimidinyl]amino]-5-thiazolecarboxamide;
′N-(2-Chloro-6-methylphenyl)-2-[[6-[[[3-(4-morpholinyl)propyl]-amino]methyl]-4-pyrimidinyl]amino]-5-thiazolecarboxamide;
′N-(2-Chloro-6-methylphenyl)-2-[[6-[[[3-(2-oxo-1-pyrrolidinyl)propyl]amino]methyl]-4-pyrimidinyl]amino]-5-thiazolecarboxamide;
′N-(2-Chloro-6-methylphenyl)-2-[[6-[[(2-1H-imidazol-4-ylethyl)amino]methyl]-4-pyrimidinyl]amino]-5-thiazolecarboxamide;
′N-(2-Chloro-6-methylphenyl)-2-[[6-[[(3-1H-imidazol-1-ylpropyl)amino]methyl]-4-pyrimidinyl]amino]-5-thiazolecarboxamide;
′N-(2-Chloro-6-methylphenyl)-2-[[6-[[[2-(2-pyridinyl)ethyl]amino]methyl]-4-pyrimidinyl]amino]-5-thiazolecarboxamide;
′N-(2-Chloro-6-methylphenyl)-2-[[6-[[[2-(3-pyridinyl)ethyl]amino]methyl]-4-pyrimidinyl]amino]-5-thiazolecarboxamide;
′1-[[6-[[5-[[(2-Chloro-6-methylphenyl)amino]carbonyl]-2-thiazolyl]amino]-4-pyrimidinyl]methyl]-4-piperidinecarboxamide;
′2-[[6-[[[2-(Acetylamino)ethyl]amino]methyl]-4-pyrimidinyl]amino]-N-(2-chloro-6-methylphenyl)-5-thiazolecarboxamide;
′N-(2-Chloro-6-methylphenyl)-2-(2-naphthalenylamino)-5-thiazolecarboxamide;
′N-(2-Chloro-6-methylphenyl)-2-(2-quinolinylamino)-5-thiazolecarboxamide;
′N-(2-Chloro-6-methylphenyl)-2-(3-isoquinolinylamino)-5-thiazolecarboxamide;
′N-(2-Chloro-6-methylphenyl)-2-(2-quinoxalinylamino)-5-thiazolecarboxamide;
′N-(2-Chloro-6-methylphenyl)-4-methyl-2-[[2-methyl-6-(4-morpholinyl)-4-pyrimidinyl]amino]-5-thiazolecarboxamide;
′N-(2-Chloro-6-methylphenyl)-4-methyl-2-[[2-methyl-6-[[2-(4-morpholinyl)ethyl]amino]-4-pyrimidinyl]amino]-5-thiazolecarboxamide;
′2-[(2,6-Dimethyl-4-pyrimidinyl)amino]-N-phenyl-5-thiazolecarboxamide;
′2-[(2,6-Dimethyl-4-pyrimidinyl)methylamino]-N-(2-methylphenyl)-5-thiazolecarboxamide;
′2-[(2,6-Dimethyl-4-pyrimidinyl)amino]-N-(2-methylphenyl)-5-thiazolecarboxamide;
′N-(3,5-Dimethoxyphenyl)-2-[(2,6-dimethyl-4-pyrimidinyl)amino]-5-thiazolecarboxamide;
′N-[2,6-Bis(1-methylethyl)phenyl]-2-[(2,6-dimethyl-4-pyrimidinyl)amino]-5-thiazolecarboxamide;
′N-(2-Chloro-6-methylphenyl)-2-[2,6-dimethyl-4-pyrimidinyl)methylamino]-5-thiazolecarboxamide;
′N-(2-Chloro-6-methylphenyl)-2-[(2,6-dimethyl-4-pyrimidinyl)amino]-N-methyl-5-thiazolecarboxamide;
N-, N-(2-Chloro-6-methylphenyl)-(4-methoxybenzyl)-2-[(6-bromo-2-pyridinyl)amino]-5-thiazolecarboxamide;
N-(2-Chloro-6-methylphenyl)-2-[(6-bromo-2-pyridinyl)amino-5-thiazolecarboxamide;
′N-(2-Chloro-6-methylphenyl)-2-[[6-[4-(2-furanylcarbonyl)-1-piperazinyl]-2-pyridinyl]amino]-5-thiazolecarboxamide;
′2-[[6-[[3-(1H-Benzimidazol-1-yl)propyl]amino]-2-pyridinyl]amino]-N-(2-chloro-6-methylphenyl)-5-thiazolecarboxamide;
′N-(2-Chloro-6-methylphenyl)-2-[[6-[[4-(1H-imidazol-1-yl)butyl]amino]-2-pyridinyl]amino]-5-thiazolecarboxamide;
′N-(2-Chloro-6-methylphenyl)-2-[[6-[[5-(1H-imidazol-1-yl)pentyl]amino]-2-pyridinyl]amino]-5-thiazolecarboxamide;
′N-(2-Chloro-6-methylphenyl)-2-[[6-[[3-(4-methyl-1-piperazinyl)propyl]amino]-2-pyridinyl]amino]-5-thiazolecarboxamide;
′N-(2-Chloro-6-methylphenyl)-2-[[6-[[4-(1H-imidazol-1-yl)phenyl]amino]-2-pyridinyl]amino]-5-thiazolecarboxamide;
′N-(2-Chloro-6-methylphenyl)-2-[[6-[[6-(1H-imidazol-1-yl)hexyl]amino]-2-pyridinyl]amino]-5-thiazolecarboxamide;
′N-(2-Chloro-6-methylphenyl)-2-[[6-[(3-1H-imidazol-1-ylpropyl)amino]-2-pyridinyl]amino]-5-thiazolecarboxamide;
′2-[[6-[[3-(1H-Imidazol-1-yl)propyl]amino]-2-pyridinyl]amino]-N-(4-methoxyphenyl)-5-thiazolecarboxamide;
′2-[[6-[[3-(1H-Imidazol-1-yl)propyl]amino]-2-pyridinyl]amino]-N-(4-phenoxyphenyl)-5-thiazolecarboxamide;
′N-(4-Chlorophenyl)-2-[[6-[[3-(1H-imidazol-1-yl)propyl]amino]-2-pyridinyl]amino]-5-thiazolecarboxamide;
′2-[[6-[[3-(1H-Imidazol-1-yl)propyl]amino]-2-pyridinyl]amino]-N-[1-(phenylmethyl)-1H-indazol-5-yl]-5-thiazolecarboxamide;
′N-(2-Ethylphenyl)-2-[[6-[[3-(1H-imidazol-1-yl)propyl]amino]-2-pyridinyl]amino]-5-thiazolecarboxamide;
′N-(2,6-Dimethoxyphenyl)-2-[[6-[[3-(1H-imidazol-1-yl)propyl]amino]-2-pyridinyl]amino]-5-thiazolecarboxamide;
′N-(2,4-Dimethoxyphenyl)-2-[[6-[[3-(1H-imidazol-1-yl)propyl]amino]-2-pyridinyl]amino]-5-thiazolecarboxamide;
′2-[[6-[[3-(1H-Imidazol-1-yl)propyl]amino]-2-pyridinyl]amino]-N-phenyl-5-thiazolecarboxamide;
′2-[[6-[[3-(1H-Imidazol-1-yl)propyl]amino]-2-pyridinyl]amino]-N-(2-methylphenyl)-5-thiazolecarboxamide;
′N-(2-Chlorophenyl)-2-[[6-[[3-(1H-imidazol-1-yl)propyl]amino]-2-pyridinyl]amino]-5-thiazolecarboxamide;
′N-(2,6-Diethylphenyl)-2-[[6-[[3-(1H-imidazol-1-yl)propyl]amino]-2-pyridinyl]amino]-5-thiazolecarboxamide;
′N-(2-Chloro-6-methylphenyl)-2-[[6-[[3-(1H-imidazol-1-yl)propyl]amino]-2-pyridinyl]amino]-4-methyl-5-thiazolecarboxamide;
′2-[[6-[[3-(1H-Imidazol-1-yl)propyl]amino]-2-pyridinyl]amino]-4-methyl-N-[1-(phenylmethyl)-1H-indazol-5-yl]-5-thiazolecarboxamide;
′N-(2-Chloro-6-methylphenyl)-2-[[3-[[3-(1H-imidazol-1-yl)propyl]amino]phenyl]amino]-5-thiazolecarboxamide;
′N-(2-Chloro-6-methylphenyl)-2-[[5-[[3-(1H-imidazol-1-yl)propyl]amino]-2-nitrophenyl]amino]-5-thiazolecarboxamide;
N-(2-Chloro-6-methylphenyl)-2-[(3,4,5-trimethoxy-phenyl)amino]-5-thiazolecarboxamide;
N-(2-Chloro-6-methyl-phenyl)-2-[(4-methoxy-phenyl)amino]-5-thiazolecarboxamide;
N-(2-Chloro-6-methyl-phenyl)-2-[(3-methoxy-phenyl)amino]-5-thiazolecarboxamide;
N-(2-Chloro-6-methyl-phenyl)-2-[(2-methoxy-phenyl)amino]-5-thiazolecarboxamide;
N-(2-Chloro-6-methyl-phenyl)-2-[(3,5-dimethoxyphenyl)amino]-5-thiazolecarboxamide;
N-(2-Chloro-6-methyl-phenyl)-2-[[4-(dimethylamino)-phenyl]amino]-5-thiazolecarboxamide;
N-(2-Chloro-6-methylphenyl)-2-[[4-(4-morpholinyl)phenyl]amino]-5-thiazolecarboxamide;
N-(2-Chloro-6-methylphenyl)-2-[[3-(carboxymethyl)-phenyl]amino]-5-thiazolecarboxamide;
N-(2-Chloro-6-methylphenyl)-2-[[3-(3-carboxypropyl)-phenyl]amino]-5-thiazolecarboxamide;
N-(2-Chloro-6-methylphenyl)-2-[[4-(carboxymethyl)phenyl]amino]-5-thiazolecarboxamide;
N-(2-Chloro-6-methylphenyl)-2-[(2-methyl-1H-benzimidazol-5-yl)amino]-5-thiazolecarboxamide;
N-(2-Chloro-6-methylphenyl)-2-[[1-[3-(1H-imidazol-1-yl)propyl]-1H-benzimidazol-4-yl]amino]-5-thiazolecarboxamide;
N-(2-Chloro-6-methylphenyl)-2-[[1-[2-(1H-imidazol-1-yl)ethyl]-1H-indazol-6-yl]amino]-5-thiazolecarboxamide;
N-(2-Chloro-6-methylphenyl)-2-[[2-[2-(1H-imidazol-1-yl)ethyl]-2H-indazol-6-yl]amino]-5-thiazolecarboxamide;
N-(2-Chloro-6-methylphenyl)-2-[(1-methyl-1H-benzimidazol-6-yl)amino]-5-thiazolecarboxamide;
N-(2-Chloro-6-methylphenyl)-2-[(1-methyl-1H-benzimidazol-5-yl)amino]-5-thiazolecarboxamide;
N-(2-Chloro-6-methylphenyl)-2-[[2-[3-(1H-imidazol-1-yl)propyl]amino]-1H-benzimidazol-5-yl]amino]-5-thiazolecarboxamide;
N-(2-Chloro-6-methylphenyl)-2-[[2-(4-morpholinylmethyl)-1H-benzimidazol-5-yl]amino]-5-thiazolecarboxamide;
N-(2-Chloro-6-methylphenyl)-2-[[2-(1H-imidazol-1-ylmethyl)-1H-benzimidazol-5-yl]amino]-5-thiazolecarboxamide;
N-(2-Chloro-6-methylphenyl)-2-[[3-[[5-(1H-imidazol-1-yl)-2-pyridinyl]amino]phenyl]amino]-5-thiazolecarboxamide;
N-(2-Chloro-6-methylphenyl)-2-[[3-[3-(1H-imidazol-1-yl)propoxy]phenyl]amino]-5-thiazolecarboxamide;
N-(2-Chloro-6-methylphenyl)-2-[[4-[3-(1H-imidazol-1-yl)propoxy]phenyl]amino]-5-thiazolecarboxamide;
N-(2-Chloro-6-methylphenyl)-2-[[3-[[[3-(1H-imidazol-1-yl)propyl]amino]sulfonyl]phenyl]amino]-5-thiazolecarboxamide; and
N-(2-Chloro-6-methylphenyl)-2-[[4-[[[3-(1H-imidazol-1-yl)propyl]amino]sulfonyl]phenyl]amino]-5-thiazolecarboxamide.

9. The method according to claim 2 wherein said anticancer agent is a MEK inhibitor having the formula: 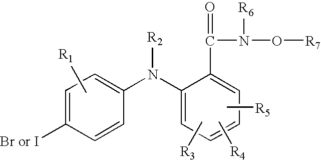
wherein
R₁ is hydrogen, hydroxy, C₁-C₈ alkyl, C₁-C₈ alkoxy, halo, trifluoromethyl, or CN;
R₂ is hydrogen;
R₃, R₄, and R₅ independently are hydrogen, hydroxy, halo, trifluoromethyl, C₁-C₈ alkyl, C₁-C₈ alkoxy, nitro, CN, or (O or NH)ₘ—(CH₂)ₙ—R₉, where R₉ is hydrogen, hydroxy, CO₂H or NR₁₀R₁₁;
n is 0 to 4;
m is 0 or 1;
R₁₀ and R₁₁, independently are hydrogen or C₁-C₈ alkyl, or taken together with the nitrogen to which they are attached can complete a 3- to 10-member cyclic ring optionally containing one, two, or three additional heteroatoms selected from O, S, NH, or N—C₁-C₈ alkyl;
R₆ is hydrogen, C₁-C₈ alkyl, 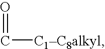
alkyl, aryl, aralkyl, or C₃-C₁₀ cycloalkyl;
R₇ is hydrogen, C₁-C₈ alkyl, C₂-C₈ alkenyl, C₂-C₈ alkynyl, C₃-C₁₀ (cycloalkyl or cycloalkyl optionally containing a heteroatom selected from O, S, or NR₉); and
wherein any of the foregoing alkyl, alkenyl, and alkynyl groups can be unsubstituted or substituted by cycloalkyl (or cycloalkyl optionally containing a heteroatom selected from O, S, or NR₉), aryl, aryloxy, heteroaryl, or heteroaryloxy; or R₆ and R₇ taken together with the N-0 to which they are attached can complete a 5- to 10-membered cyclic ring, optionally containing one, two, or three additional heteroatoms selected from O, S, or NR₁₀R₁₁.

10. The method according to claim 9 wherein said MEK inhibitor is selected from the group consisting of:
4-Fluoro-N-hydroxy-2-(4-iodo-2-methyl-phenylamino)-benzamide;
4-Fluoro-2-(4-iodo-2-methyl-phenylamino)-N-(methoxy)-benzamide;
4-Fluoro-2-(4-iodo-2-methyl-phenylamino)-N-(prop-2-ynyloxy)-benzamide;
4-Fluoro-2-(4-iodo-2-methyl-phenylamino)-N-(2-phenoxyethoxy)-benzamide;
4-Fluoro-2-(4-iodo-2-methyl-phenylamino)-N-(2-thienylmethoxy)-benzamide;
4-Fluoro-2-(4-iodo-2-methyl-phenylamino)-N-(prop-2-enyloxy)-benzamide;
4-Fluoro-2-(4-iodo-2-methyl-phenylamino)-N-(cyclopropylmethoxy)-benzamide;
4-Fluoro-2-(4-iodo-2-methyl-phenylamino)-N-(cyclopentoxy)-benzamide;
4-Fluoro-N-hydroxy-2-(4-iodo-2-methyl-phenylamino)-N-isopropyl-benzamide; and
4-Fluoro-N-hydroxy-2-(4-iodo-2-methyl-phenylamino)-N-methyl-benzamide.
3,4-Difluoro-2-(4-iodo-2-methyl-phenylamino)-N-(3-furylmethoxy)-benzamide;
3,4-Difluoro-2-(4-iodo-2-methyl-phenylamino)-N-ethoxy-benzamide;
3,4-Difluoro-2-(4-iodo-2-methyl-phenylamino)-N-(but-2-enyloxy)-benzamide;
3,4-Difluoro-2-(4-iodo-2-methyl-phenylamino)-N-(cyclopropyl-methoxy)-benzamide;
3,4-Difluoro-2-(4-iodo-2-methyl-phenylamino)-N-(1-methylprop-2-ynyloxy)-benzamide;
3,4-Difluoro-2-(4-iodo-2-methyl-phenylamino)-N-(3-phenylprop-2-ynyloxy)-benzamide;
3,4-Difluoro-2-(4-iodo-2-methyl-phenylamino)-N-(3-methyl-5-phenylpent-2-en-4-ynyloxy)-benzamide;
3,4-Difluoro-2-(4-iodo-2-methyl-phenylamino)-N-(prop-2-ynyloxy)-benzamide;
3,4-Difluoro-2-(4-iodo-2-methyl-phenylamino)-N-(propoxy)-benzamide;
3,4-Difluoro-2-(4-iodo-2-methyl-phenylamino)-N-(cyclobutoloxy)-benzamide;
3,4-Difluoro-2-(4-iodo-2-methyl-phenylamino)-N-(2-thienylmethoxy)-benzamide;
3,4-Difluoro-2-(4-iodo-2-methyl-phenylamino)-N-(2-methyl-prop-2-enyloxy)-benzamide;
3,4-Difluoro-2-(4-iodo-2-methyl-phenylamino)-N-(2-phenoxyethoxy)-benzamide;
3,4-Difluoro-2-(4-iodo-2-methyl-phenylamino)-N-(but-2-enyloxy)-benzamide;
3,4-Difluoro-2-(4-iodo-2-methyl-phenylamino)-N-(but-3-ynyloxy)-benzamide;
3,4-Difluoro-2-(4-iodo-2-methyl-phenylamino)-N-(cyclopentyloxy)-benzamide;
3,4-Difluoro-2-(4-iodo-2-methyl-phenylamino)-N-(3-(2-fluorophenyl)-prop-2-ynyloxy)-benzamide;
3,4-Difluoro-2-(4-iodo-2-methyl-phenylamino)-N-(tetrahydro-pyran-2-yloxy)-benzamide;
3,4-Difluoro-N-hydroxy-2-(4-iodo-2-methyl-phenylamino)-benzamide;
3,4-Difluoro-2-(2-chloro-4-iodo-phenylamino)-N-cyclobutylmethoxy-benzamide;
3,4-Difluoro-2-(2-chloro-4-iodo-phenylamino)-N-(tetrahydro-pyran-2-yloxy)-benzamide; and
3,4-Difluoro-2-(2-chloro-4-iodo-phenylamino)-N-cyclopropylmethoxy-benzamide.

11. The method according to claim 2 wherein said anticancer agent is a pan HER inhibitor.

12. The method according to claim 11 wherein said pan HER inhibitor has the formula: 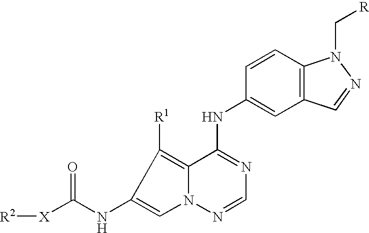
wherein
R is selected from the group consisting of aryl, substituted aryl, heterocyclo, and substituted heterocyclo;
R¹ is selected from the group consisting of alkyl and substituted alkyl;
R² is selected from the group consisting of hydrogen, alkyl, substituted alkyl, cycloalkyl, substituted cycloalkyl, aryl, substituted aryl, aralkyl, heterocyclo, and substituted heterocyclo; or, R² may be absent;
X is selected from the group consisting of a bond, O, S, C(R³)₂, C(R³)₃, NR³; and N(R³)₂;
R³ is independently selected from the group consisting of hydrogen, alkyl, substituted alkyl, aryl, substituted aryl, aralkyl, heterocyclo, and substituted heterocyclo,
and pharmaceutically acceptable salts, prodrugs, enantiomers, diastereomers, and solvates thereof.

13. The method according to claim 11 wherein said pan HER inhibitor has the formula: 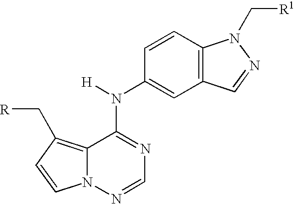
its enantiomers, diastereomers, and pharmaceutically acceptable salts, prodrugs and solvates thereof, wherein
R¹ is selected from the group consisting of SR², SOR², SO₂R², OR², and NR³R⁴;
R¹ is selected from the group consisting of aryl, substituted aryl, heterocyclo, and substituted heterocyclo;
R² is selected from the group consisting of hydrogen, alkyl, substituted alkyl, aryl, substituted aryl, aralkyl, heterocyclo, and substituted heterocyclo;
R³ and R⁴ are independently selected from the group consisting of hydrogen, alkyl, substituted alkyl, aryl, substituted aryl, heterocyclo, and substituted heterocyclo;
or R² and R³ may together form an optionally substituted monocyclic 4-8 membered saturated or unsaturated carbocyclic or heterocyclic ring, or an optionally substituted bicyclic 7 to 12 membered saturated or unsaturated carbocyclic or heterocyclic ring.

14. The method according to claim 2 wherein said IGF1R inhibitor has the formula I: 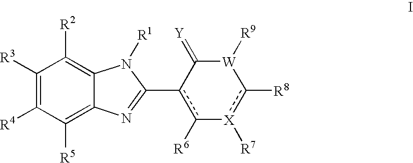
and includes enantiomers, diastereomers, pharmaceutically acceptable salts, hydrates, prodrugs and solvates thereof:
wherein
X is N, C₁-C₃ alkyl, or a direct bond;
Y is O or S;
W is N, C, O, or S; provided that if W is O or S, R⁹ is absent;
R¹ is H, alkyl, or alkoxy;
R² and R⁹ are independently H or alkyl;
R³ is H, C₁₋₆ alkyl, alkenyl, alkynyl, cycloalkyl, heterocycloalkyl, halo, amino, —OR⁶⁰, —NO₂, —OH, —SR⁶⁰, —NR⁶⁰R⁶¹, —CN, —C(O)R⁶⁰, —CO₂R⁶⁰, —CONR⁶⁰R⁶¹, OCONR⁶⁰R⁶¹, —NR⁶²CONR⁶⁰R⁶¹, —NR⁶⁰SO₂R⁶¹, —SO₂NR⁶⁰R⁶¹, —SO₂R⁶³, —C(NR⁶²)NR⁶⁰R⁶¹, —C(NH⁶²)-morpholine, aryl, heteroaryl, —(CH₂)ₙC(O)₂—R⁶⁰, —NR⁶⁰R⁶¹—(CH₂)ₙOR⁶⁰, —(CH₂)ₙNR⁶⁰R⁶¹, —(CH₂)ₙSR⁶⁰, —(CH₂)ₙ aryl, —(CH₂)ₙ heteroaryl, or —(CH₂)ₙ heterocycloalkyl, wherein n is 1 to 3:
R⁴ is H, halo, alkyl or haloalkyl;
R⁵ is H, alkyl, halo, or aryl;
R⁶, R⁷, and R⁸ are each independently —NH-Z-aryl or —NH-Z-heteroaryl wherein Z is C₁-C₄ alkyl, alkenyl, or alkynyl; Z optionally having one or more hydroxy, thiol, alkoxy, thioalkoxy, amino, halo, NR⁶⁰SO₂R⁶¹ groups; Z optionally incorporating one or more groups selected from the group consisting of CO, CNOH, CNOR⁶⁰, CNNR⁶⁰, CNNCOR⁶⁰ and CNNSO₂R⁶⁰;
R⁶⁰, R⁶¹, R⁶², and R⁶³ are independently selected from the group consisting of H, alkyl, alkenyl, alkynyl, cycloalkyl, cycloalkylalkyl, hydroxy, alkoxy, aryl, heteroaryl, heteroarylalkyl, and alkyl-R²⁵;
R²⁵ is hydrogen, alkenyl, hydroxy, thiol, alkoxy, thioalkoxy, amino, alkylamino, dialkylamino, aryl, heteroaryl, cyano, halo, sulfoxy, sulfonyl, —NR³⁰COOR³¹, —NR³⁰C(O)R³¹, —NR³⁰SO₂R³¹, —C(O)NR³⁰R³¹, heteroaryl or heterocycloalkyl; and
R³⁰ and R³¹ are, independently, hydrogen, alkyl, or cycloalkyl.

15. The method according to claim 14 wherein R³ is an optionally substituted morpholine, thiomorpholine, sulfoxymorpholine, sulfonylmorpholine, or homomorpholine.

16. The method according to claim 14 wherein R³ is a substituted or unsubstituted piperazine or piperadine.

17. The method according to claim 14 wherein R⁶ is —NH-Z-aryl, or —NH-Z-heteroaryl.

18. The method according to claim 17 wherein said aryl is a substituted or unsubstituted phenyl.

19. The method according to claim 17 wherein said heteroaryl is a substituted or unsubstituted pyridinyl, imidazolyl, pyrazolyl, pyrrolyl or triazolyl.

20. The method according to claim 2 wherein the anticancer agent is cetuximab, erlotinib, or gefitinib and the IGF1R inhibitor is of the following formula: 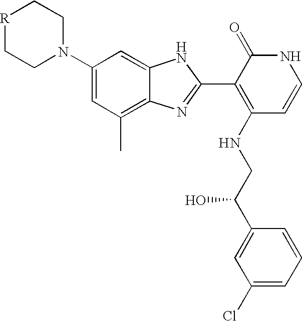
wherein R is —NCH₂CH₂OMe or O.

21. The method according to claim 1 wherein the anticancer agent is a microtubule-affecting agent; a natural product or derivative thereof, or a platinum coordination complex.

22. The method according to claim 21 wherein said microtubule-affecting agent is allocolchicine, Halichondrin B, colchicine, colchicine derivatives, dolastatin 10, maytansine, rhizoxin, paclitaxel, a paclitaxel derivative, thiocolchicine, trityl cysteine, vinblastine sulfate, vincristine sulfate, epothilone A, epothilone B, discodermolide, estramustine, nocodazole, or MAP4.

23. The method according to claim 21 wherein said natural product is a vinca alkaloid, an antitumor antibiotic, an enzyme, lymphokine, epipodophyllotoxin, Vinblastine, Vincristine, Vindesine, Bleomycin, Dactinomycin, Daunorubicin, Doxorubicin, Epirubicin, Idarubicin, Ara-C, Mithramycin, Deoxyco-formycin, Mitomycin-C, L-Asparaginase, an Interferon, Etoposide, or Teniposide.

24. The method according to claim 21 wherein said platinum coordination complex is cisplatin or carboplatin.

25 The method according to claim 23 wherein said anticancer agent is etoposide.

26. The method according to claim 21 wherein said anticancer agent is paclitaxel.

27. The method according to claim 21 wherein said IGF1R inhibitor has the formula I: 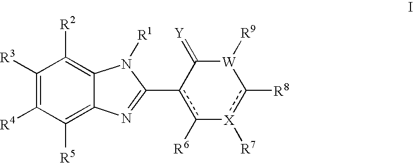
and includes enantiomers, diastereomers, pharmaceutically acceptable salts, hydrates, prodrugs and solvates thereof:
wherein
X is N, C₁-C₃ alkyl, or a direct bond;
Y is O or S;
W is N, C, O, or S; provided that if W is O or S, R⁹ is absent;
R¹ is H, alkyl, or alkoxy;
R² and R⁹ are independently H or alkyl;
R³ is H, C₁₋₆ alkyl, alkenyl, alkynyl, cycloalkyl, heterocycloalkyl, halo, amino, —OR⁶⁰, —NO₂, —OH, —SR⁶⁰, —NR⁶⁰R⁶¹, —CN, —C(O)R⁶⁰, —CO₂R⁶⁰, —CONR⁶⁰R⁶¹, OCONR⁶⁰R⁶¹, —NR⁶²CONR⁶⁰R⁶¹, —NR⁶⁰SO₂R⁶¹, —SO₂NR⁶⁰R⁶¹, —SO₂R⁶³, —C(NR⁶²)NR⁶⁰R⁶¹, —C(NH⁶²)-morpholine, aryl, heteroaryl, —(CH₂)ₙC(O)₂—R⁶⁰, —NR⁶⁰R⁶¹—(CH₂)ₙOR⁶⁰, —(CH₂)ₙNR⁶⁰R⁶¹, —(CH₂)ₙSR⁶⁰, —(CH₂)ₙ aryl, —(CH₂)ₙ heteroaryl, or —(CH₂)ₙ heterocycloalkyl, wherein n is 1 to 3:
R⁴ is H, halo, alkyl or haloalkyl;
R⁵ is H, alkyl, halo, or aryl;
R⁶, R⁷, and R⁸ are each independently —NH-Z-aryl or —NH-Z-heteroaryl wherein Z is C₁-C₄ alkyl, alkenyl, or alkynyl; Z optionally having one or more hydroxy, thiol, alkoxy, thioalkoxy, amino, halo, NR⁶⁰SO₂R⁶¹ groups; Z optionally incorporating one or more groups selected from the group consisting of CO, CNOH, CNOR⁶⁰, CNNR⁶⁰, CNNCOR⁶⁰ and CNNSO₂R⁶⁰;
R⁶⁰, R⁶¹R⁶², and R⁶³ are independently selected from the group consisting of H, alkyl, alkenyl, alkynyl, cycloalkyl, cycloalkylalkyl, hydroxy, alkoxy, aryl, heteroaryl, heteroarylalkyl, and alkyl-R²⁵;
R²⁵ is hydrogen, alkenyl, hydroxy, thiol, alkoxy, thioalkoxy, amino, alkylamino, dialkylamino, aryl, heteroaryl, cyano, halo, sulfoxy, sulfonyl, —NR³⁰COOR³¹, —NR³⁰C(O)R³¹, —NR³⁰SO₂R³¹, —C(O)NR³⁰R³¹, heteroaryl or heterocycloalkyl; and
R³⁰ and R³¹ are, independently, hydrogen, alkyl, or cycloalkyl.

28. The method according to claim 27 wherein said anticancer agent is etoposide, paclitaxel, or cisplatin.

29. The method according to claim 28 wherein the IGF1R inhibitor has the formula: 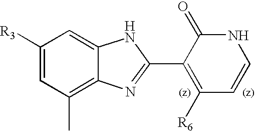
wherein R³ is an unsubstituted or substituted morpholine, piperazine or piperidine and R⁶ is —NHZ-aryl or —NHZ-heteroaryl.

30. The method according to claim 28 wherein said IGF1R inhibitor has the formula: 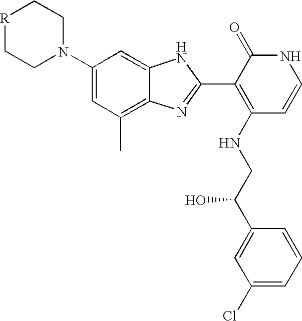
wherein R is —NCH₂CH₂OMe or O.
